SEQUENCE LISTING (90 sequences)

SEQ ID NO: 1 - DNA, 2325 bp, Artificial Sequence
  source: /note=“Description of Artificial Sequence: Synthetic polynucleotide”
atggcccaggtgagcaagcagacctccaagaagagggagctgagcatcgacgagtaccagggcgcccggaagtggtgcttcaccattgccttcaacaaggccctggtgaaccgggacaagaacgacggcctgttcgtggaaagcctgctgagacacgagaagtacagcaagcacgactggtacgacgaagatacccgggccctgatcaagtgcagcacccaggccgccaacgccaaggctgaagccctgcggaactacttcagtcactaccggcatagccctggctgcctgaccttcaccgccgaggacgaactgcggaccatcatggagagagcctatgagcgggccatcttcgagtgcagaagaagagagacagaggtgatcatcgagtttcccagcctgttcgagggcgaccggatcaccaccgccggcgtggtgtttttcgtgagctttttcgtggaaagaagagtgctggatcggctgtatggagccgtgtccggcctgaagaagaatgagggacagtacaagctgacccggaaggccctgagcatgtactgcctgaaggacagcagattcaccaaggcctgggataagcgggtgctgctgttcagagacatcctggcccagctgggaagaatccccgccgaggcctacgagtactaccacggcgagcagggtgataagaagagagctaacgacaatgagggcacaaatcccaagcggcacaaggacaagttcatcgaatttgcactgcactacctggaagcccagcacagcgagatctgcttcggcagacgccacatcgtgcgggaagaggccggcgccggcgatgagcacaagaagcaccggaccaagggaaaggtggtggtggacttcagcaagaaggacgaggaccagagctactatatctccaagaacaacgtgatcgtgcggatcgacaagaacgccggccctagaagctaccggatgggcctgaacgagctgaagtacctcgtgctgctgagcctgcaggggaagggcgacgatgccatcgccaagctgtacagatacagacagcacgtggagaacatcctggatgtggtgaaggtgaccgataaggataaccacgtgttcctgccccgcttcgtgctggagcagcacggcatcggcagaaaggccttcaagcagcggatcgatggacgggtgaagcacgtgcggggcgtgtgggagaagaagaaggccgccaccaatgaaatgaccctgcacgagaaggccagagacatcctgcagtacgtgaacgaaaactgcacccggtccttcaaccctggcgaatacaacagactgctggtgtgcctggtgggcaaggacgtggagaactttcaggccggcctgaagcggctgcagctggccgaaaggatcgatggccgggtgtactccatcttcgcccagaccagcaccatcaatgagatgcaccaggtggtgtgcgaccagatcctgaaccggctgtgcagaatcggcgaccagaagctgtacgattacgtgggactgggcaagaaggacgaaatcgactacaagcagaaggtggcctggttcaaggagcacatcagcatccggagaggattcctgagaaagaagttctggtacgatagcaagaagggattcgcaaagctggtggaggaacacctggagtccggcggcggccagcgcgacgtgggcctggacaagaagtactaccacatcgacgccatcggcagattcgagggcgccaaccccgccctgtacgagaccctggccagagatcggctgtgcctcatgatggcccagtacttcctgggcagcgtgagaaaggaactgggcaacaagattgtgtggagcaacgacagcatcgaactgcctgtggaaggctctgtgggaaatgagaagagcatcgtgttctccgtgtctgactacggcaagctgtacgtgctggacgatgccgaattcctgggccggatctgcgaatacttcatgccccacgaaaagggcaagatccggtaccacacagtgtacgaaaagggctttagagcatacaacgacctgcagaagaagtgcgtggaggccgtgctggctttcgaagagaaggtggtgaaggccaagaagatgagcgagaaggaaggcgcccactacatcgacttccgggagatcctggcccagaccatgtgcaaggaggccgaaaagaccgcagtgaacaaggtgagacgcgccttcttccaccaccacctgaagttcgtgattgacgagttcggcctgttcagcgacgtgatgaagaagtacggcatcgagaaggaatggaagttccctgtcaag

SEQ ID NO: 2 - protein, 775 aa, Artificial Sequence
  source: /note=“Description of Artificial Sequence: Synthetic polypeptide”
  MOD_RES (672)..(672): Any amino acid
  MOD_RES (676)..(676): Any amino acid
  MOD_RES (751)..(751): Any amino acid
  source: /note=“See specification as filed for detailed description of substitutions and preferred embodiments”
MAQVSKQTSKKRELSIDEYQGARKWCFTIAFNKALVNRDKNDGLFVESLLRHEKYSKHDWYDEDTRALIKCSTQAANAKAEALRNYFSHYRHSPGCLTFTAEDELRTIMERAYERAIFECRRRETEVIIEFPSLFEGDRITTAGVVFFVSFFVERRVLDRLYGAVSGLKKNEGQYKLTRKALSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPVEGSVGNEKSIVFSVSDYGKLYVLDDAEFLGRICEYFMPHEKGKIRXHTVXEKGFRAYNDLQKKCVEAVLAFEEKVVKAKKMSEKEGAHYIDFREILAQTMCKEAEKTAVNKVRRAFFHHHLKFVXDEFGLFSDVMKKYGIEKEWKFPVK

SEQ ID NO: 3 - protein, 775 aa, Artificial Sequence
  source: /note=“Description of Artificial Sequence: Synthetic polypeptide”
MAQVSKQTSKKRELSIDEYQGARKWCFTIAFNKALVNRDKNDGLFVESLLRHEKYSKHDWYDEDTRALIKCSTQAANAKAEALRNYFSHYRHSPGCLTFTAEDELRTIMERAYERAIFECRRRETEVIIEFPSLFEGDRITTAGVVFFVSFFVERRVLDRLYGAVSGLKKNEGQYKLTRKALSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPVEGSVGNEKSIVFSVSDYGKLYVLDDAEFLGRICEYFMPHEKGKIRAHTVAEKGFRAYNDLQKKCVEAVLAFEEKVVKAKKMSEKEGAHYIDFREILAQTMCKEAEKTAVNKVRRAFFHHHLKFVIDEFGLFSDVMKKYGIEKEWKFPVK

SEQ ID NO: 4 - protein, 775 aa, Unknown
  source: /note=“Description of Unknown: Cas13e sequence”
MAQVSKQTSKKRELSIDEYQGARKWCFTIAFNKALVNRDKNDGLFVESLLRHEKYSKHDWYDEDTRALIKCSTQAANAKAEALRNYFSHYRHSPGCLTFTAEDELRTIMERAYERAIFECRRRETEVIIEFPSLFEGDRITTAGVVFFVSFFVERRVLDRLYGAVSGLKKNEGQYKLTRKALSMYCLKDSRFTKAWDKRVLLFRDILAQLGRIPAEAYEYYHGEQGDKKRANDNEGTNPKRHKDKFIEFALHYLEAQHSEICFGRRHIVREEAGAGDEHKKHRTKGKVVVDFSKKDEDQSYYISKNNVIVRIDKNAGPRSYRMGLNELKYLVLLSLQGKGDDAIAKLYRYRQHVENILDVVKVTDKDNHVFLPRFVLEQHGIGRKAFKQRIDGRVKHVRGVWEKKKAATNEMTLHEKARDILQYVNENCTRSFNPGEYNRLLVCLVGKDVENFQAGLKRLQLAERIDGRVYSIFAQTSTINEMHQVVCDQILNRLCRIGDQKLYDYVGLGKKDEIDYKQKVAWFKEHISIRRGFLRKKFWYDSKKGFAKLVEEHLESGGGQRDVGLDKKYYHIDAIGRFEGANPALYETLARDRLCLMMAQYFLGSVRKELGNKIVWSNDSIELPVEGSVGNEKSIVFSVSDYGKLYVLDDAEFLGRICEYFMPHEKGKIRYHTVYEKGFRAYNDLQKKCVEAVLAFEEKVVKAKKMSEKEGAHYIDFREILAQTMCKEAEKTAVNKVRRAFFHHHLKFVIDEFGLFSDVMKKYGIEKEWKFPVK

SEQ ID NO: 5 - DNA, 2325 bp, Artificial Sequence
  source: /note=“Description of Artificial Sequence: Synthetic polynucleotide”
atggcccaggtgagcaagcagacctccaagaagagggagctgagcatcgacgagtaccagggcgcccggaagtggtgcttcaccattgccttcaacaaggccctggtgaaccgggacaagaacgacggcctgttcgtggaaagcctgctgagacacgagaagtacagcaagcacgactggtacgacgaagatacccgggccctgatcaagtgcagcacccaggccgccaacgccaaggctgaagccctgcggaactacttcagtcactaccggcatagccctggctgcctgaccttcaccgccgaggacgaactgcggaccatcatggagagagcctatgagcgggccatcttcgagtgcagaagaagagagacagaggtgatcatcgagtttcccagcctgttcgagggcgaccggatcaccaccgccggcgtggtgtttttcgtgagctttttcgtggaaagaagagtgctggatcggctgtatggagccgtgtccggcctgaagaagaatgagggacagtacaagctgacccggaaggccctgagcatgtactgcctgaaggacagcagattcaccaaggcctgggataagcgggtgctgctgttcagagacatcctggcccagctgggaagaatccccgccgaggcctacgagtactaccacggcgagcagggtgataagaagagagctaacgacaatgagggcacaaatcccaagcggcacaaggacaagttcatcgaatttgcactgcactacctggaagcccagcacagcgagatctgcttcggcagacgccacatcgtgcgggaagaggccggcgccggcgatgagcacaagaagcaccggaccaagggaaaggtggtggtggacttcagcaagaaggacgaggaccagagctactatatctccaagaacaacgtgatcgtgcggatcgacaagaacgccggccctagaagctaccggatgggcctgaacgagctgaagtacctcgtgctgctgagcctgcaggggaagggcgacgatgccatcgccaagctgtacagatacagacagcacgtggagaacatcctggatgtggtgaaggtgaccgataaggataaccacgtgttcctgccccgcttcgtgctggagcagcacggcatcggcagaaaggccttcaagcagcggatcgatggacgggtgaagcacgtgcggggcgtgtgggagaagaagaaggccgccaccaatgaaatgaccctgcacgagaaggccagagacatcctgcagtacgtgaacgaaaactgcacccggtccttcaaccctggcgaatacaacagactgctggtgtgcctggtgggcaaggacgtggagaactttcaggccggcctgaagcggctgcagctggccgaaaggatcgatggccgggtgtactccatcttcgcccagaccagcaccatcaatgagatgcaccaggtggtgtgcgaccagatcctgaaccggctgtgcagaatcggcgaccagaagctgtacgattacgtgggactgggcaagaaggacgaaatcgactacaagcagaaggtggcctggttcaaggagcacatcagcatccggagaggattcctgagaaagaagttctggtacgatagcaagaagggattcgcaaagctggtggaggaacacctggagtccggcggcggccagcgcgacgtgggcctggacaagaagtactaccacatcgacgccatcggcagattcgagggcgccaaccccgccctgtacgagaccctggccagagatcggctgtgcctcatgatggcccagtacttcctgggcagcgtgagaaaggaactgggcaacaagattgtgtggagcaacgacagcatcgaactgcctgtggaaggctctgtgggaaatgagaagagcatcgtgttctccgtgtctgactacggcaagctgtacgtgctggacgatgccgaattcctgggccggatctgcgaatacttcatgccccacgaaaagggcaagatccgggcccacacagtggccgaaaagggctttagagcatacaacgacctgcagaagaagtgcgtggaggccgtgctggctttcgaagagaaggtggtgaaggccaagaagatgagcgagaaggaaggcgcccactacatcgacttccgggagatcctggcccagaccatgtgcaaggaggccgaaaagaccgcagtgaacaaggtgagacgcgccttcttccaccaccacctgaagttcgtgattgacgagttcggcctgttcagcgacgtgatgaagaagtacggcatcgagaaggaatggaagttccctgtcaag

SEQ ID NO: 6 - DNA, 36 bp, Artificial Sequence
  source: /note=“Description of Artificial Sequence: Synthetic oligonucleotide”
gctggagcagcccccgatttgtggggtgattacagc

SEQ ID NO: 7 - DNA, 30 bp, Artificial Sequence
  source: /note=“Description of Artificial Sequence: Synthetic oligonucleotide”
gtgctgtaggaagctcatctctcctatgtg

SEQ ID NO: 8 - DNA, 30 bp, Artificial Sequence
  source: /note=“Description of Artificial Sequence: Synthetic oligonucleotide”
ggtactcctggaagatgtccaccagggtct

SEQ ID NO: 9 - skipped

SEQ ID NO: 10 - DNA, 141 bp, Artificial Sequence
  source: /note=“Description of Artificial Sequence: Synthetic polynucleotide”
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 11 - DNA, 141 bp, Artificial Sequence
  source: /note=“Description of Artificial Sequence: Synthetic polynucleotide”
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 12 - DNA, 261 bp, Artificial Sequence
  source: /note=“Description of Artificial Sequence: Synthetic polynucleotide”
taggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagggttgg

SEQ ID NO: 13 - DNA, 9 bp, Artificial Sequence
  source: /note=“Description of Artificial Sequence: Synthetic oligonucleotide”
gccaccatg

SEQ ID NO: 14 - DNA, 21 bp, Artificial Sequence
  source: /note=“Description of Artificial Sequence: Synthetic oligonucleotide”
cccaagaagaagcggaaggtg

SEQ ID NO: 15 - DNA, 135 bp, Artificial Sequence
  source: /note=“Description of Artificial Sequence: Synthetic polynucleotide”
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 16 - DNA, 241 bp, Artificial Sequence
  source: /note=“Description of Artificial Sequence: Synthetic polynucleotide”
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 17 - DNA, 3569 bp, Artificial Sequence
  source: /note=“Description of Artificial Sequence: Synthetic polynucleotide”
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccatcgatggcgcgccggattcgacattgattattgataggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagggttggaccggtgccaccatgcccaagaagaagcggaaggtggcccaggtgagcaagcagacctccaagaagagggagctgagcatcgacgagtaccagggcgcccggaagtggtgcttcaccattgccttcaacaaggccctggtgaaccgggacaagaacgacggcctgttcgtggaaagcctgctgagacacgagaagtacagcaagcacgactggtacgacgaagatacccgggccctgatcaagtgcagcacccaggccgccaacgccaaggctgaagccctgcggaactacttcagtcactaccggcatagccctggctgcctgaccttcaccgccgaggacgaactgcggaccatcatggagagagcctatgagcgggccatcttcgagtgcagaagaagagagacagaggtgatcatcgagtttcccagcctgttcgagggcgaccggatcaccaccgccggcgtggtgtttttcgtgagctttttcgtggaaagaagagtgctggatcggctgtatggagccgtgtccggcctgaagaagaatgagggacagtacaagctgacccggaaggccctgagcatgtactgcctgaaggacagcagattcaccaaggcctgggataagcgggtgctgctgttcagagacatcctggcccagctgggaagaatccccgccgaggcctacgagtactaccacggcgagcagggtgataagaagagagctaacgacaatgagggcacaaatcccaagcggcacaaggacaagttcatcgaatttgcactgcactacctggaagcccagcacagcgagatctgcttcggcagacgccacatcgtgcgggaagaggccggcgccggcgatgagcacaagaagcaccggaccaagggaaaggtggtggtggacttcagcaagaaggacgaggaccagagctactatatctccaagaacaacgtgatcgtgcggatcgacaagaacgccggccctagaagctaccggatgggcctgaacgagctgaagtacctcgtgctgctgagcctgcaggggaagggcgacgatgccatcgccaagctgtacagatacagacagcacgtggagaacatcctggatgtggtgaaggtgaccgataaggataaccacgtgttcctgccccgcttcgtgctggagcagcacggcatcggcagaaaggccttcaagcagcggatcgatggacgggtgaagcacgtgcggggcgtgtgggagaagaagaaggccgccaccaatgaaatgaccctgcacgagaaggccagagacatcctgcagtacgtgaacgaaaactgcacccggtccttcaaccctggcgaatacaacagactgctggtgtgcctggtgggcaaggacgtggagaactttcaggccggcctgaagcggctgcagctggccgaaaggatcgatggccgggtgtactccatcttcgcccagaccagcaccatcaatgagatgcaccaggtggtgtgcgaccagatcctgaaccggctgtgcagaatcggcgaccagaagctgtacgattacgtgggactgggcaagaaggacgaaatcgactacaagcagaaggtggcctggttcaaggagcacatcagcatccggagaggattcctgagaaagaagttctggtacgatagcaagaagggattcgcaaagctggtggaggaacacctggagtccggcggcggccagcgcgacgtgggcctggacaagaagtactaccacatcgacgccatcggcagattcgagggcgccaaccccgccctgtacgagaccctggccagagatcggctgtgcctcatgatggcccagtacttcctgggcagcgtgagaaaggaactgggcaacaagattgtgtggagcaacgacagcatcgaactgcctgtggaaggctctgtgggaaatgagaagagcatcgtgttctccgtgtctgactacggcaagctgtacgtgctggacgatgccgaattcctgggccggatctgcgaatacttcatgccccacgaaaagggcaagatccgggcccacacagtggccgaaaagggctttagagcatacaacgacctgcagaagaagtgcgtggaggccgtgctggctttcgaagagaaggtggtgaaggccaagaagatgagcgagaaggaaggcgcccactacatcgacttccgggagatcctggcccagaccatgtgcaaggaggccgaaaagaccgcagtgaacaaggtgagacgcgccttcttccaccaccacctgaagttcgtgattgacgagttcggcctgttcagcgacgtgatgaagaagtacggcatcgagaaggaatggaagttccctgtcaagcccaagaagaagcggaaggtgtagaagcttaacttgtttattgcagcttataatggttacaaataaagcaatagcatcacaaatttcacaaataaagcatttttttcactgcattctagttgtggtttgtccaaactcatcaatgtatcttatcatgtctggatcactagtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggctggagcagcccccgatttgtggggtgattacagcgtgctgtaggaagctcatctctcctatgtggctggagcagcccccgatttgtggggtgattacagcggtactcctggaagatgtccaccagggtctgctggagcagcccccgatttgtggggtgattacagctttttttacgcgttagttattaatagtgcgtcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 18-19 - skipped (2 entries)

SEQ ID NO: 20 - protein, 7 aa, Simian virus 40
PKKKRKV

SEQ ID NO: 21 - protein, 16 aa, Unknown
  source: /note=“Description of Unknown: Nucleoplasmin bipartite NLS sequence”
KRPAATKKAGQAKKKK

SEQ ID NO: 22 - protein, 9 aa, Unknown
  source: /note=“Description of Unknown: C-myc NLS sequence”
PAAKRVKLD

SEQ ID NO: 23 - protein, 11 aa, Unknown
  source: /note=“Description of Unknown: C-myc NLS sequence”
RQRRNELKRSP

SEQ ID NO: 24 - protein, 38 aa, Homo sapiens
NQSSNFGPMKGGNFGGRSSGPYGGGGQYFAKPRNQGGY

SEQ ID NO: 25 - protein, 42 aa, Unknown
  source: /note=“Description of Unknown: IBB domain from importin-alpha sequence”
RMRIZFKNKGKDTAELRRRRVEVSVELRKAKKDEQILKRRNV

SEQ ID NO: 26 - protein, 8 aa, Unknown
  source: /note=“Description of Unknown: Myoma T protein sequence”
VSRKRPRP

SEQ ID NO: 27 - protein, 8 aa, Unknown
  source: /note=“Description of Unknown: Myoma T protein sequence”
PPKKARED

SEQ ID NO: 28 - protein, 8 aa, Homo sapiens
PQPKKKPL

SEQ ID NO: 29 - protein, 5 aa, Influenza virus
DRLRR

SEQ ID NO: 30 - protein, 7 aa, Influenza virus
PKQKKRK

SEQ ID NO: 31 - protein, 10 aa, Hepatitis delta virus
RKLKKKIKKL

SEQ ID NO: 32 - protein, 10 aa, Mus musculus
REKKKFLKRR

SEQ ID NO: 33 - protein, 20 aa, Homo sapiens
KRKGDEVDGVDEVAKKKSKK

SEQ ID NO: 34 - protein, 17 aa, Homo sapiens
RKCLQAGMNLEARKTKK

SEQ ID NO: 35 - protein, 6 aa, Homo sapiens
PKLKRQ

SEQ ID NO: 36 - protein, 4 aa, Homo sapiens
RPRK

SEQ ID NO: 37 - protein, 8 aa, Chicken anemia virus
RRARRPRG

SEQ ID NO: 38 - protein, 21 aa, Homo sapiens
GKRKLITSEEERSPAKRGRKS

SEQ ID NO: 39 - protein, 22 aa, Homo sapiens
KGKKGRTQKEKKAARARSKGKN

SEQ ID NO: 40 - protein, 28 aa, Homo sapiens
RKRCAAGVGGGPAGCPAPGSTPLKKPRR

SEQ ID NO: 41 - protein, 36 aa, Unknown
  source: /note=“Description of Unknown: NLS ERK5 sequence”
RKPVTAQERQREREEKRRRRQERAKEREKRRQERER

SEQ ID NO: 42 - protein, 27 aa, Saccharomyces cerevisiae
RSGGNHRRNGRGGRGGYNRRNNGYHPY

SEQ ID NO: 43 - protein, 27 aa, Human betaherpesvirus 5
TLLLRETMNNLGVSDHAVLSRKTPQPY

SEQ ID NO: 44 - protein, 18 aa, Homo sapiens
PGKMDKGEHRQERRDRPY

SEQ ID NO: 45 - protein, 21 aa, Homo sapiens
GKKKKGKPGKRREQRKKKRRT

SEQ ID NO: 46 - protein, 27 aa, Saccharomyces cerevisiae
SANKVTKNKSNSSPYLNKRKGKPGPDS

SEQ ID NO: 47 - protein, 43 aa, Homo sapiens
VHSHKKKKIPTSPTFTTPKTLTLRRQPKYPRKSAPRRNKLDHY

SEQ ID NO: 48 - protein, 10 aa, Homo sapiens
RKHKTNRKPR

SEQ ID NO: 49 - DNA, 6660 bp, Artificial Sequence
  source: /note=“Description of Artificial Sequence: Synthetic polynucleotide”
gaattcactggccgtcgttttacaacgtcgtgactgggaaaaccctggcgttacccaacttaatcgccttgcagcacatccccctttcgccagctggcgtaatagcgaagaggcccgcaccgatggatcccgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcttggcccggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaaaacaatagcaggcatgctggggatgcggtgggctctatggaagctagcagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcgccataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcgccatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgccgcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggccattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaaggccgccgctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcgccacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagaggcccgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcgccagactcgaaagcgtagctacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctctcgagatctaggtttacgacaag

SEQ ID NO: 50 - DNA, 508 bp, Artificial Sequence
  source: /note=“Description of Artificial Sequence: Synthetic polynucleotide”
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 51 - DNA, 711 bp, Artificial Sequence
  source: /note=“Description of Artificial Sequence: Synthetic polynucleotide”
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 52 - DNA, 225 bp, Artificial Sequence
  source: /note=“Description of Artificial Sequence: Synthetic polynucleotide”
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaaaacaatagcaggcatgctggggatgcggtgggctctatgg

SEQ ID NO: 53 - protein, 12 aa, Mus musculus
SALIKKKKKMAP

SEQ ID NO: 54 - protein, 6 aa, Unknown
  source: /note=“Description of Unknown: NLS-RARalpha sequence”
RNKKKK

SEQ ID NO: 55 - protein, 6 aa, Artificial Sequence
  source: /note=“Description of Artificial Sequence: Synthetic peptide”
  VARIANT (3)..(3): /replace=“F”
  VARIANT (4)..(4): /replace=“Y”
  SITE (1)..(6): /note=“Variant residues given in the sequence have no preference with respect to those in the annotations for variant positions”
RNYFSH

SEQ ID NO: 56 - protein, 6 aa, Artificial Sequence
  source: /note=“Description of Artificial Sequence: Synthetic peptide”
RNYFSH

SEQ ID NO: 57 - protein, 6 aa, Artificial Sequence
  source: /note=“Description of Artificial Sequence: Synthetic peptide”
RNFYSH

SEQ ID NO: 58 - protein, 6 aa, Artificial Sequence
  source: /note=“Description of Artificial Sequence: Synthetic peptide”
  VARIANT (3)..(3): /replace=“K”
  SITE (1)..(6): /note=“Variant residues given in the sequence have no preference with respect to those in the annotations for variant positions”
RNAALH

SEQ ID NO: 59 - protein, 6 aa, Artificial Sequence
  source: /note=“Description of Artificial Sequence: Synthetic peptide”
RAFFHH

SEQ ID NO: 60 - protein, 6 aa, Artificial Sequence
  source: /note=“Description of Artificial Sequence: Synthetic peptide”
RRAFFH

SEQ ID NO: 61 - protein, 7 aa, Artificial Sequence
  source: /note=“Description of Artificial Sequence: Synthetic peptide”
GSGGGGS

SEQ ID NO: 62 - protein, 15 aa, Artificial Sequence
  source: /note=“Description of Artificial Sequence: Synthetic peptide”
GGGGSGGGGSGGGGS

SEQ ID NO: 63 - protein, 336 aa, Unknown
  source: /note=“Description of Unknown: ADAR1DD-WT sequence”
SLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNF

SEQ ID NO: 64 - protein, 336 aa, Artificial Sequence
  source: /note=“Description of Artificial Sequence: Synthetic polypeptide”
SLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGQGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNF

SEQ ID NO: 65 - protein, 385 aa, Unknown
  source: /note=“Description of Unknown: ADAR2DD-WT sequence”
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 66 - protein, 385 aa, Artificial Sequence
  source: /note=“Description of Artificial Sequence: Synthetic polypeptide”
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 67 - protein, 198 aa, Unknown [sequence omitted]

SEQ ID NO: 68 - protein, 208 aa, Unknown
  source: /note=“Description of Unknown: Lamprey_AID-APOBEC1 sequence”
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 69 - protein, 229 aa, Unknown [sequence omitted]

SEQ ID NO: 70 - RNA, 36 nt, Artificial Sequence
  source: /note=“Description of Artificial Sequence: Synthetic oligonucleotide”
ggcccaacaugaggaucacccaugucugcaggggcc

SEQ ID NO: 71 - RNA, 29 nt, Artificial Sequence
  source: /note=“Description of Artificial Sequence: Synthetic oligonucleotide”
ggcccaugcugucuaagacagcaugggcc

SEQ ID NO: 72 - RNA, 34 nt, Artificial Sequence
  source: /note=“Description of Artificial Sequence: Synthetic oligonucleotide”
ggcccuaaggguuuauauggaaacccuuagggcc

SEQ ID NO: 73 - DNA, 21 bp, Artificial Sequence
  source: /note=“Description of Artificial Sequence: Synthetic primer”
gagggcagaatcatcacgaag

SEQ ID NO: 74 - DNA, 22 bp, Artificial Sequence
  source: /note=“Description of Artificial Sequence: Synthetic primer”
gtgaggtttgatccgcataatc

SEQ ID NO: 75 - DNA, 20 bp, Artificial Sequence
  source: /note=“Description of Artificial Sequence: Synthetic primer”
ttggtggagcgatttgtctg

SEQ ID NO: 76 - DNA, 20 bp, Artificial Sequence
  source: /note=“Description of Artificial Sequence: Synthetic primer”
gaatggggttcaacgggtta

SEQ ID NO: 77 - DNA, 27 bp, Artificial Sequence
  source: /note=“Description of Artificial Sequence: Synthetic primer”
gtgctgtaggaagctcatctctcctat

SEQ ID NO: 78 - DNA, 20 bp, Artificial Sequence
  source: /note=“Description of Artificial Sequence: Synthetic primer”
gaagatgtccaccagggtct

SEQ ID NO: 79 - DNA, 20 bp, Artificial Sequence
  source: /note=“Description of Artificial Sequence: Synthetic primer”
gctactgccgtccgattgag

SEQ ID NO: 80 - DNA, 20 bp, Artificial Sequence
  source: /note=“Description of Artificial Sequence: Synthetic primer”
cactccagggcttcatcgtt

SEQ ID NO: 81 - DNA, 24 bp, Artificial Sequence
  source: /note=“Description of Artificial Sequence: Synthetic probe”
tccaggagtaccccgacgagatag

SEQ ID NO: 82 - DNA, 20 bp, Artificial Sequence
  source: /note=“Description of Artificial Sequence: Synthetic primer”
cggcgagcagggtgataaga

SEQ ID NO: 83 - DNA, 21 bp, Artificial Sequence
  source: /note=“Description of Artificial Sequence: Synthetic primer”
ccaggtagtgcagtgcaaatt

SEQ ID NO: 84 - DNA, 24 bp, Artificial Sequence
  source: /note=“Description of Artificial Sequence: Synthetic probe”
tccttgtgccgcttgggatttgtg

SEQ ID NO: 85 - DNA, 20 bp, Artificial Sequence
  source: /note=“Description of Artificial Sequence: Synthetic primer”
ggtactcctggaagatgtcc

SEQ ID NO: 86 - DNA, 20 bp, Artificial Sequence
  source: /note=“Description of Artificial Sequence: Synthetic primer”
tgtaatcaccccacaaatcg

SEQ ID NO: 87 - DNA, 22 bp, Artificial Sequence
  source: /note=“Description of Artificial Sequence: Synthetic probe”
accagggtctgctggagcagcc

SEQ ID NO: 88 - DNA, 11 bp, Artificial Sequence
  source: /note=“Description of Artificial Sequence: Synthetic oligonucleotide”
cctgcaggcag

SEQ ID NO: 89 - DNA, 10 bp, Artificial Sequence
  source: /note=“Description of Artificial Sequence: Synthetic oligonucleotide”
ttaattaagg

SEQ ID NO: 90 - DNA, 11 bp, Artificial Sequence
  source: /note=“Description of Artificial Sequence: Synthetic oligonucleotide”
aaagcccgggc

(19) United States
(12) Patent Application Publication
(10) Pub. No.: US 2023/0075045 A1
(43) Pub. Date: Mar. 9, 2023

(54) ENGINEERED CRISPR/CAS13 SYSTEM AND USES THEREOF

(71) Applicant: HuiGene Therapeutics Co., Ltd., Shanghai (CN)

(72) Inventors: Xing Wang, Shanghai (CN); Shaoran Wang, Shanghai (CN); Xuan Yao, Shanghai (CN); Ming Mei, Shanghai (CN); Linyu Shi, Shanghai (CN)

(21) Appl. No.: 17/836,266

(22) Filed: Jun. 9, 2022

Related U.S. Application Data
(63) Continuation of application No. PCT/CN2022/079890, filed on Mar. 9, 2022.

(30) Foreign Application Priority Data
Mar. 9, 2021 (CN) PCT/CN2021/079821
Aug. 22, 2021 (CN) PCT/CN2021/113929
Sep. 29, 2021 (CN) PCT/CN2021/121926

(51) Int. Cl. A61K 49/00; C12N 15/86; C12N 9/22; C12N 15/11; C12N 15/90; A61K 48/00; A61K 31/7088; A61P 27/02

(52) U.S. Cl. CPC A61K 49/0008; C12N 15/86; C12N 9/22; C12N 15/11; C12N 15/907; A61K 48/0058; A61K 48/0066; A61K 31/7088; A61P 27/02; C12N 2310/20; C12N 2800/80; C12N 2750/14143; C12N 2750/14145

(57) ABSTRACT

The invention provides novel engineered CRISPR/Cas effector enzymes, such as Cas13 (e.g., Cas13e) that substantially maintain guide-sequence-specific endonuclease activity and substantially lack guide-sequence-independent collateral endonuclease activity compared to the corresponding wild-type Cas. Also provided are polynucleotides encoding the same, vectors or host cells comprising the polynucleotides or engineered Cas, and method of use, such as in RNA-based target gene transcript knock down.

### REFERENCE TO RELATED APPLICATIONS

The instant application is a continuation application, filed under 35 U.S.C. 111(a), of International Patent Application No. PCT/CN2022/079890, filed on Mar. 9, 2022, which claims foreign priority under 35 U.S.C. 365(b), to International Patent Application No. PCT/CN2021/121926, filed on Sep. 29, 2021, International Patent Application No. PCT/CN2021/113929, filed on Aug. 22, 2021, and International Patent Application No. PCT/CN2021/079821, filed on Mar. 9, 2021, the entire contents of each of the above-referenced applications, including any sequence listing and drawings, are incorporated herein by reference.

### SEQUENCE LISTING

The instant application contains a Sequence Listing which has been submitted electronically in ASCII format and is hereby incorporated by reference in its entirety. Said ASCII copy, created on Jun. 9, 2022, is named 132045-00801_SL.txt and is 81,255 bytes in size.

### BACKGROUND OF THE INVENTION

Age-related macular degeneration (AMD), the leading cause of visual dysfunction for adults over age 50, is a degenerative condition in the macular area of the retina and can blur central vision. There are 2 types of AMD, non-neovascular form (dry AMD) and neovascular form (wetAMD), account for 90% and 10% of total AMD, respectively. Dry AMD progresses slowly over several years characterized by formation of drusen, or yellowish deposits of extracellular material and change of retinal pigment epithelium. There is no treatment for dry AMD. The wetAMD occurs when abnormal blood vessels grow into the macula and leak blood or fluid which leads to scarring of the macula and characterized by the development of choroidal neovascularization (CNV). wetAMD can cause rapid vision loss and accounts for 90% blindness of AMD. An angiogenic growth factor vascular endothelial growth factor A (VEGFA) plays a crucial role in CNV pathogenesis.

VEGFA produced in the retina and induced by hypoxia and other conditions increases retinal vascular permeability. Anti-VEGFA therapy using humanized antibodies has been widely used in treating wetAMD, with the therapeutic effects maintained by regular injections of antibodies. Despite the burden of repeated dosing, it was reported that visual acuity decline was observed over the ensuing yeas likely due to a combination of under-treatment, incomplete treatment effectiveness, and progression of fibrotic scarring and/or geographic atrophy. More efficient therapy strategy is needed.

### SUMMARY OF THE INVENTION

One aspect of the invention provide a recombinant adeno-associate virus (rAAV) vector genome, comprising: (1) a Cas13X polynucleotide (such as SEQ ID NO: 5) encoding a Cas13X polypeptide, said Cas13X polynucleotide being at least 80%, 85%, 90%, 95%, 96%, 97%, 98%, 99%, 99.2%, 99.4%, 99.6%, 99.8%, or 99.9% identical to SEQ ID NO: 1, said Cas13X polypeptide comprising 1-3 substitutions at Y672, Y676, and/or I751 of SEQ ID NO: 4 (the wt protein encoded by SEQ ID NO: 1), and having substantially the same (e.g., at least about 80%, 90%, 95%, 99% or more) guide RNA-specific nuclease activity as SEQ ID NO: 4 and substantially no (e.g., at most 20%, 15%, 10%, 5%) collateral (guide RNA-independent) nuclease activity of SEQ ID NO: 4; and, (2) a polyA signal sequence 3′ to the Cas13X polynucleotide; optionally, said Cas13X polypeptide has the amino acid sequence of SEQ ID NO: 2 or 3.

In certain embodiments, the rAAV vector genome comprises a 5′ AAV ITR sequence and a 3′ AAV ITR sequence.

In certain embodiments, the 5′ and the 3′ AAV ITR sequences are both wild-type AAV ITR sequences from AAV1, AAV2, AAV3A, AAV3B, AAV4, AAV5, AAV6, AAV7, AAVrh74, AAV8, AAV9, AAV10, AAV11, AAV12, AAV13, or a member of the Clade to which any of the AAV1-AAV13 belong, or a functional truncated variant thereof (such as SEQ ID NO: 10 or 11).

In certain embodiments, the rAAV vector genome further comprises a promoter operably linked to and drives the transcription from the Cas13X polynucleotide. In certain embodiments, the promoter is a ubiquitous promoter. In certain embodiments, the promoter is a tissue-specific promoter. In certain embodiments, the promoter is a constitutive promoter. In certain embodiments, the promoter is an inducible promoter.

In certain embodiments, the promoter is selected from the group consisting of a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter.

In certain embodiments, the promoter is the elongation factor 1α short (EFS) promoter, such as SEQ ID NO: 12.

In certain embodiments, the rAAV vector genome further comprises a coding sequence for a nuclear localization sequence (NLS) fused N-terminal, C-terminal, or internally to the Cas13X polypeptide, and/or a or a coding sequence for a nuclear export signal (NES) fused N-terminal, C-terminal, or internally to the Cas13X polypeptide.

In certain embodiments, the rAAV vector genome comprises a first NLS coding sequence 5′ to the Cas13X polynucleotide, and/or a second NLS coding sequence 3′ to the Cas13X polynucleotide (e.g., comprising both the first and the second NLS coding sequences). In certain embodiments, the NLS, the first NLS, and the second NLS is independently selected from the group consisting of SEQ ID NOs: 20-48 or 53-54.

In certain embodiments, rAAV vector genome further comprises a Kozak sequence or a functional variant thereof (such as SEQ ID NO: 13).

In certain embodiments, the rAAV vector genome further comprises a polyadenylation (polyA) signal sequence. In certain embodiments, the polyA signal sequence is selected from the group consisting of growth hormone polyadenylation signal (bGH polyA), a small polyA signal (SPA), a human growth hormone polyadenylation signal (hGH polyA), a SV40 polyA signal (SV40 polyA), a rabbit beta globin polyA signal (rBG polyA), or a variant thereof. In certain embodiments, the polyA signal sequence is SV40 polyA signal sequence or a functional variant thereof (such as SEQ ID NO: 15).

In certain embodiments, the rAAV genome vector further comprises a second transcription unit comprising an RNA pol III promoter, wherein said second transcription unit is 3′ to the Cas13X polynucleotide.

In certain embodiments, the RNA pol III promoter is U6 (such as SEQ ID NO: 16), H1, 7SK, or a variant thereof.

In certain embodiments, the said second transcription unit further comprises a second coding sequence operably linked to the RNA pol III promoter, encoding one or more single guide RNAs (sgRNAs) each complementary to a target RNA sequence, and each capable of directing said Cas13X polypeptide to cleave said target RNA; optionally, each said sgRNA comprises a direct repeat (DR) sequence that binds the Cas13X polypeptide.

In certain embodiments, the DR sequence is a nucleic sequence having at least 90% identity to SEQ ID NO: 6, at most 1, 2, 3, 4, or 5 nucleotide differences from SEQ ID NO: 6, and/or substantially the same secondary structure as that of SEQ ID NO: 6. In certain embodiments, the DR sequence comprises, consists essentially of, or consists of SEQ ID NO: 6.

In certain embodiments, the target RNA is a transcript (e.g., mRNA) of a target gene associated with an eye disease or disorder.

In certain embodiments, the eye disease or disorder is amoebic keratitis, fungal keratitis, bacterial keratitis, viral keratitis, onchorcercal keratitis, keratoconjunctivitis, bacterial keratoconjunctivitis, viral keratoconjunctivitis, vernal keratoconjunctivitis, atopic keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, noninfectious uveitis, anterior uveitis, posterior uveitis (including toxoplasmosis), pan-uveitis, an inflammatory disease of the vitreous or retina, endophthalmitis prophylaxis and treatment, macular edema, macular degeneration, wet age related macular degeneration (wet AMD), dry age related macular degeneration (dry AMD), diabetic macular edema (DME), allergic conjunctivitis, proliferative and non-proliferative diabetic retinopathy, hypertensive retinopathy, an autoimmune disease of the retina, primary and metastatic intraocular melanoma, other intraocular metastatic tumors, open angle glaucoma, Stargardt's disease, Fundus Flavimaculatus, closed angle glaucoma, pigmentary glaucoma, retinitis pigmentosa (RP), Leber's congenital amaurosis (LCA), Usher's syndrome, choroideremia, a rod-cone or cone-rod dystrophy, a ciliopathy, a mitochondrial disorder, progressive retinal atrophy, a degenerative retinal disease, geographic atrophy, a familial or acquired maculopathy, a retinal photoreceptor disease, a retinal pigment epithelial-based disease, cystoid macular edema, retinal detachment, traumatic retinal injury, iatrogenic retinal injury, macular holes, macular telangiectasia, a ganglion cell disease, an optic nerve cell disease, optic neuropathy, ischemic retinal disease, retinopathy of prematurity, retinal vascular occlusion, familial macroaneurysm, a retinal vascular disease, an ocular vascular diseases, a vascular disease, an ischemic optic neuropathy disease, diabetic retinal oedema, senile macular degeneration due to sub-retinal neovascularization, myopic retinopathy, retinal ischemia, choroidal vascular insufficiency, choroidal thrombosis and neovascular retinopathies resulting from carotoid artery ischemia, corneal neovascularisation, a corneal disease or opacification with an exudative or inflammatory component, diffuse lamellar keratitis, neovascularisation due to penetration of the eye or contusive ocular injury, rubosis iritis, Fuchs' heterochromic iridocyclitis, chronic uveitis, anterior uveitis, inflammatory conditions resulting from surgeries such as LASIK, LASEK, refractive surgery, IOL implantation; irreversible corneal oedema as a complication of cataract surgery, oedema as a result of insult or trauma, inflammation, infectious and non-infectious conjunctivitis, iridocyclitis, iritis, scleritis, episcleritis, superficial punctuate keratitis, keratoconus, posterior polymorphous dystrophy, Fuch's dystrophies, aphakic and pseudophakic bullous keratopathy, corneal oedema, scleral disease, ocular cicatrcial pemphigoid, pars planitis, Posner Schlossman syndrome, Behcet's disease, Vogt-Koyanagi-Harada syndrome, hypersensitivity reactions, ocular surface disorders, conjunctival oedema, Toxoplasmosis chorioretinitis, inflammatory pseudotumor of the orbit, chemosis, conjunctival venous congestion, periorbiatal cellulits, acute dacroycystitis, non-specific vasculitis, sarcoidosis, cytomegalovirus infection, and combinations thereof.

In certain embodiments, the eye disease or disorder is wet age related macular degeneration (wet AMD).

In certain embodiments, the target gene is selected from the group consisting of Vascular Endothelial Growth Factor A (VEGFA), complement factor H (CFH), age-related maculopathy susceptibility 2 (ARMS2), HtrA serine peptidase 1 (HTRA1), ATP Binding Cassette Subfamily A Member 4 (ABCA4), Peripherin-2 (PRPH2), fibulin-5 (FBLN5), ERCC Excision Repair 6 Chromatin Remodeling Factor (ERCC6), Retina And Anterior Neural Fold Homeobox 2 (RAX2), Complement C3 (C3), Toll Like Receptor 4 (TLR4), Cystatin C (CST3), CX3C Chemokine Receptor 1 (CX3CR1), complement factor I (CFI), Complement C2 (C2), Complement Factor B (CFB), Complement C9 (C9), Mitochondrially Encoded TRNA Leucine 1 (UUA/G) (MT-TL-1), Complement Factor H Related 1 (CFHR1), Complement Factor H Related 3 (CFHR3), Ciliary Neurotrophic Factor (CNTF), pigment epithelium-derived factor (PEDF), rod-derived cone viability factor (RdCVF), glial-derived neurotrophic factor (GDNF), Myosin VIIA (MYO7A); Centrosomal Protein 290 (CEP290), Cadherin Related 23 (CDH23), Eyes Shut Homolog (EYS), Usherin (USH2A), adhesion G protein-coupled receptor V1 (ADGRV1), ALMS1 Centrosome And Basal Body Associated Protein (ALMS1), Retinoid Isomerohydrolase 65 kDa (RPE65), Aryl-hydrocarbon-interacting protein-like 1 (AIPL1), Guanylate Cyclase 2D, Retinal (GUCY2D), Leber Congenital Amaurosis 5 Protein (LCA5), Cone-Rod Homeobox (CRX), Clarin (CLRN1), ATP Binding Cassette Subfamily A Member 4 (ABCA4), Retinol Dehydrogenase 12 (RDH12), Inosine Monophosphate Dehydrogenase 1 (IMPDH1), Crumbs Cell Polarity Complex Component 1 (CRB1), Lecithin retinol acyltransferase (LRAT), Nicotinamide Nucleotide Adenylyltransferase 1 (NMNAT1), TUB Like Protein 1 (TULP1), MER Proto-Oncogene, Tyrosine Kinase (MERTK), Retinitis Pigmentosa GTPase Regulato (RPGR), RP2 Activator Of ARL3 GTPase (RP2), X-linked retinitis pigmentosa GTPase regulator-interacting protein 1 (RPGRIP), Cyclic Nucleotide Gated Channel Subunit Alpha 3 (CNGA3), Cyclic Nucleotide Gated Channel Subunit Beta 3 (CNGB3), G Protein Subunit Alpha Transducin 2 (GNAT2), Fibroblast Growth Factor 2 (FGF2), Erythropoietin (EPO), BCL2 Apoptosis Regulator (BCL2), BCL2 Like 1 (BCL2L1), Nuclear Factor Kappa B (NFκB), Endostatin, Angiostatin, fins-like tyrosine kinase receptor (sFlt), Pigment-dispersing factor receptor (Pdfr), Interleukin 10 (IL10), soluble interleukin 17 (sIL17R), Interleukin-1-receptor antagonist (IL1-ra), TNF Receptor Superfamily Member 1A (TNFRSF1A), TNF Receptor Superfamily Member 1B (TNFRSF1B), and interleukin 4 (IL4).

In certain embodiments, the target gene is VEGFA.

In certain embodiments, the target RNA is a transcript (e.g., mRNA) of a target gene associated with a neurodegenerative disease or disorder.

In certain embodiments, the neurodegenerative disease or disorder is alcoholism, Alexander's disease, Alper's disease, Alzheimer's Disease, amyotrophic lateral sclerosis (ALS), ataxia telangiectasia, neuronal ceroid lipofuscinoses, Batten disease, bovine spongiform encephalopathy (BSE), Canavan disease, cerebral palsy, Cockayne syndrome, corticobasal degeneration, Creutzfeldt-Jakob disease, frontotemporal lobar degeneration, Huntington's disease, HIV-associated dementia, Kennedy's disease, Lewy body dementia, neuroborreliosis, primary age-related tauopathy (PART)/Neurofibrillary tangle-predominant senile dementia, Machado-Joseph disease, multiple system atrophy, multiple sclerosis, multiple sulfatase deficiency, mucolipidoses, narcolepsy, Niemann Pick disease, Parkinson's Disease, Pick's disease, Pompe disease, primary lateral sclerosis, prion diseases, neuronal loss, cognitive defect, motor neuron diseases, Duchenne Muscular Dystrophy (DMD), frontotemporal dementia, frontotemporal dementia and parkinsonism linked to chromosome 17, Lytico-Bodig disease (Parkinson-dementia complex of Guam), neuroaxonal dystrophies, Refsum's disease, Schilder's disease, subacute combined degeneration of spinal cord secondary to pernicious anaemia, Spielmeyer-Vogt-Sjogren-Batten disease, Parkinsonism linked to chromosome 17 (FTDP-17), Prader Willi syndrome, Myotonic dystrophy, chronic traumatic encephalopathy including dementia pugilistica, spinocerebellar ataxia, spinal muscular atrophy, Steele-Richardson-Olszewski disease, Tabes dorsalis, Niemann-Pick Type C (NPC1 and/or NPC2 defect), Smith-Lemli-Opitz Syndrome (SLOS), an inborn error of cholesterol synthesis, Tangier disease, Pelizaeus-Merzbacher disease, a neuronal ceroid lipofuscinosis, a primary glycosphingolipidosis, Farber disease or multiple sulphatase deficiency, Gaucher disease, Fabry disease, GM1 gangliosidosis, GM2 gangliosidosis, Krabbe disease, metachromatic leukodystrophy (MLD), NPC, GM1 gangliosidosis, Fabry disease, a neurodegenerative mucopolysaccharidosis, MPS I, MPS IH, MPS IS, MPS II, MPS III, MPS IIIA, MPS IIIB, MPS IIIC, MPS HID, MPS, IV, MPS IV A, MPS IV B, MPS VI, MPS VII, MPS IX, a disease with secondary lysosomal involvement, SLOS, Tangier disease, ganglioglioma, gangliocytoma, meningioangiomatosis, postencephalitic parkinsonism, subacute sclerosing panencephalitis, lead encephalopathy, tuberous sclerosis, Hallervorden-Spatz disease, lipofuscinosis, cerebellar ataxia, parkinsonism, Louis-Barr syndrome, multiple systems atrophy, fronto-temporal dementia or lower body Parkinson's syndrome, Niemann Pick disease, Niemann Pick type C, Niemann Pick type A, Tay-Sachs disease, multisystemic atrophy cerebellar type (MSA-C), fronto-temporal dementia with parkinsonism, progressive supranuclear palsy, cerebellar downbeat nystagmus, Sandhoff's disease or mucolipidosis type II, or combinations thereof.

In certain embodiments, the target RNA is a transcript (e.g., mRNA) of a target gene associated with a cancer.

In certain embodiments, the cancer is carcinomas, sarcomas, myelomas, leukemias, lymphomas and mixed type tumors. Non-limiting examples of cancers that may treated by methods and compositions described herein include, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; plasmacytoma, colorectal cancer, rectal cancer, and hairy cell leukemia.

In certain embodiments, said one or more sgRNAs comprise SEQ ID NOs: 7 and 8.

In certain embodiments, the rAAV vector genome comprises an ITR-to-ITR polynucleotide (such as SEQ ID NO: 17) comprising, from 5′ to 3′: (a) a 5′ ITR from AAV2 (such as SEQ ID NO: 10); (b) an EFS promoter (such as SEQ ID NO: 12); (c) a KOZAK sequence (such as SEQ ID NO: 13); (d) a first SV40 NLS coding sequence (such as SEQ ID NO: 14); (e) the Cas13X polynucleotide (such as SEQ ID NO: 5) encoding the Cas13X polypeptide of SEQ ID NO: 2 or 3; (f) a second SV40 NLS coding sequence (such as SEQ ID NO: 14); (g) an SV40 polyA signal sequence (such as SEQ ID NO: 15); (h) a U6 promoter (such as SEQ ID NO: 16); (i) a first direct repeat sequence (such as SEQ ID NO: 6); (j) an sg1 coding sequence specific for VEGFA (such as SEQ ID NO: 7); (k) a second direct repeat sequence (such as SEQ ID NO: 6); (l) an sg2 coding sequence specific for VEGFA (such as SEQ ID NO: 8); (m) a third direct repeat sequence (such as SEQ ID NO: 6); and, (n) a 3′ ITR from AAV2 (such as SEQ ID NO: 11); or a polynucleotide at least about 90%, 91%, 92%, 93%, 94%, 95%, 96%, 97%, 98%, 99%, 99.5%, 99.6%, 99.7%, 99.8%, 99.9% identical to said ITR-to-ITR polynucleotide.

Another aspect of the invention provides a recombinant AAV (rAAV) vector genome comprising, consisting essentially of, or consisting of SEQ ID NO: 17, or a polynucleotide at least 90%, 91%, 92%, 93%, 94%, 95%, 96%, 97%, 98%, 99%, 99.5%, 99.6%, 99.7%, 99.8%, 99.9% identical thereto, wherein said polynucleotide encodes a Cas13X polypeptide at least 80%, 85%, 90%, 95%, 96%, 97%, 98%, or 99% identical to SEQ ID NO: 4, and an sgRNA specific for VEGFA, wherein said Cas13X polypeptide comprises 1-3 substitutions at Y672, Y676, and/or I751 of SEQ ID NO: 4, and wherein said sgRNA forms a complex with said Cas13X polypeptide and directs said Cas13X polypeptide to cleave a VEGFA mRNA transcript with substantially the same (e.g., at least about 80%, 90%, 95%, 99% or more) guide RNA-specific nuclease activity as SEQ ID NO: 4 and substantially no (e.g., at most 20%, 15%, 10%, 5%) collateral (guide RNA-independent) nuclease activity of SEQ ID NO: 4.

In certain embodiments, the rAAV vector genome is SEQ ID NO: 17, or the polynucleotide at least 95% or 99% identical thereto. In certain embodiments, the rAAV vector genome is SEQ ID NO: 17.

In certain embodiments, the rAAV vector further comprising a third transcription unit with a coding sequence under the transcriptional control of a 3rd promoter.

In certain embodiments, the third transcription unit is the most 3′ transcription unit, and the coding sequence encodes a marker gene (such as mCherry) under the transcriptional control of a CMV promoter; optionally, the third transcription unit further comprises a bGH polyA signal sequence.

Another aspect of the invention provides a recombinant AAV (rAAV) viral particle comprising the rAAV vector genome of the invention.

In certain embodiments, the rAAV viral particle comprises a capsid with a serotype of AAV1, AAV2, AAV3A, AAV3B, AAV4, AAV5, AAV6, AAV7, AAVrh74, AAV8, AAV9, AAV10, AAV11, AAV12, AAV13, or a member of the Clade to which any of the AAV1-AAV13 belong. In certain embodiments, the capsid serotype is AAV9.

Another aspect of the invention provides a recombinant AAV (rAAV) viral particle comprising the rAAV vector genome of the invention, encapsidated in a capsid with a serotype of AAV9.

In certain embodiments, the rAAV viral particle comprises the rAAV vector genome of the invention.

Another aspect of the invention provides a pharmaceutical composition comprising the rAAV vector genome of the invention, or the rAAV viral particle of the invention, and a pharmaceutically acceptable excipient.

Another aspect of the invention provides a method of treating a subject having an eye disease or disorder in a subject in need thereof, the method comprising administering to the subject a therapeutically effective amount of the rAAV vector genome of the invention, the rAAV viral particle of the invention, or the pharmaceutically composition of the invention, wherein the rAAV vector genome or the rAAV viral particle specifically down-regulate the expression of a target gene causative of the eye disease or disorder.

In certain embodiments, administrating comprises contacting a cell with the therapeutically effective amount of the rAAV vector genome of the invention, the rAAV viral particle of the invention, or the pharmaceutically composition of the invention.

In certain embodiments, the cell is located in the eye of the subject.

In certain embodiments, the eye disease or disorder is amoebic keratitis, fungal keratitis, bacterial keratitis, viral keratitis, onchorcercal keratitis, keratoconjunctivitis, bacterial keratoconjunctivitis, viral keratoconjunctivitis, vernal keratoconjunctivitis, atopic keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, noninfectious uveitis, anterior uveitis, posterior uveitis (including toxoplasmosis), pan-uveitis, an inflammatory disease of the vitreous or retina, endophthalmitis prophylaxis and treatment, macular edema, macular degeneration, wet age related macular degeneration (wet AMD), dry age related macular degeneration (dry AMD), diabetic macular edema (DME), allergic conjunctivitis, proliferative and non-proliferative diabetic retinopathy, hypertensive retinopathy, an autoimmune disease of the retina, primary and metastatic intraocular melanoma, other intraocular metastatic tumors, open angle glaucoma, Stargardt's disease, Fundus Flavimaculatus, closed angle glaucoma, pigmentary glaucoma, retinitis pigmentosa (RP), Leber's congenital amaurosis (LCA), Usher's syndrome, choroideremia, a rod-cone or cone-rod dystrophy, a ciliopathy, a mitochondrial disorder, progressive retinal atrophy, a degenerative retinal disease, geographic atrophy, a familial or acquired maculopathy, a retinal photoreceptor disease, a retinal pigment epithelial-based disease, cystoid macular edema, retinal detachment, traumatic retinal injury, iatrogenic retinal injury, macular holes, macular telangiectasia, a ganglion cell disease, an optic nerve cell disease, optic neuropathy, ischemic retinal disease, retinopathy of prematurity, retinal vascular occlusion, familial macroaneurysm, a retinal vascular disease, an ocular vascular diseases, a vascular disease, an ischemic optic neuropathy disease, diabetic retinal oedema, senile macular degeneration due to sub-retinal neovascularization, myopic retinopathy, retinal ischemia, choroidal vascular insufficiency, choroidal thrombosis and neovascular retinopathies resulting from carotoid artery ischemia, corneal neovascularisation, a corneal disease or opacification with an exudative or inflammatory component, diffuse lamellar keratitis, neovascularisation due to penetration of the eye or contusive ocular injury, rubosis iritis, Fuchs' heterochromic iridocyclitis, chronic uveitis, anterior uveitis, inflammatory conditions resulting from surgeries such as LASIK, LASEK, refractive surgery, IOL implantation; irreversible corneal oedema as a complication of cataract surgery, oedema as a result of insult or trauma, inflammation, infectious and non-infectious conjunctivitis, iridocyclitis, iritis, scleritis, episcleritis, superficial punctuate keratitis, keratoconus, posterior polymorphous dystrophy, Fuch's dystrophies, aphakic and pseudophakic bullous keratopathy, corneal oedema, scleral disease, ocular cicatrcial pemphigoid, pars planitis, Posner Schlossman syndrome, Behcet's disease, Vogt-Koyanagi-Harada syndrome, hypersensitivity reactions, ocular surface disorders, conjunctival oedema, Toxoplasmosis chorioretinitis, inflammatory pseudotumor of the orbit, chemosis, conjunctival venous congestion, periorbiatal cellulits, acute dacroycystitis, non-specific vasculitis, sarcoidosis, cytomegalovirus infection, and combinations thereof. In certain embodiments, the eye disease or disorder is wet age related macular degeneration (wet AMD).

In certain embodiments, the subject is a human.

In certain embodiments, expression of the target gene in the cell is decreased in comparison to a cell having not been contacted with the rAAV vector genome of the invention, the rAAV viral particle of the invention, or the pharmaceutically composition of the invention. In certain embodiments, the target gene is VEGFA.

In certain embodiments, choroidal neovascularization (CVN) is decreased in the eye of the subject by about 30%, about 35%, about 40%, about 45%, about 50%, about 55%, about 60%, about 65%, about 70%, about 75%, about 80%, or about 85% compared to CVN prior to treatment.

In certain embodiments, the decrease of CVN in the eye of the subject is stable for at least about 2 weeks, about 3 weeks, about 4 weeks, about 5 weeks, about 6 weeks, about 7 weeks, about 8 weeks, about 9 weeks, or at least about 10 weeks.

Another aspect of the invention provides a non-human primate (NHP) model of wet AMD/CNV, comprising a NHP with an eye having developed laser-induced CNV, wherein the CNV is induced by laser photocoagulation about one month (e.g., about 3 weeks, 4 weeks, 31 days, or 5 weeks) after first administering one or more immunosuppressors to the NHP, and the laser-induced CNV persists at least about 4 weeks.

In certain embodiments, the one or more immunosuppressors are administered daily, for at least 20 days, at least 22 days, at least 24 days, at least 26 days, at least 28 days, at least 30 days, at least 32 days, at least 34 days, at least 36 days, at least 38 days, at least 40 days, at least 42 days, at least 43 days, at least 44 days, at least 46 days, at least 48 days, or at least 50 days.

In certain embodiments, the laser-induced CNV persists at least 5 weeks, at least 6 weeks, at least 7 weeks, at least 8 weeks, at least 9 weeks, or at least 10 weeks.

In certain embodiments, the one or more immunosuppressors are first administered at least 2 weeks, at least 3 weeks, at least 4 weeks, at least 5 weeks, at least 6 weeks, at least 7 weeks, or at least 8 weeks prior to laser photocoagulation.

In certain embodiments, the one or more immunosuppressors comprise triptolide, corticosteroids such as prednisone, calcineurin inhibitors such as tacrolimus (Envarsus XR® or Protopic), cyclosporine (Gengraf®, Neoral® or Sandimmune®), Inosine monophosphate dehydrogenase (IMDH) inhibitors such as mycophenolate mofetil (CellCept®), imuran (Azathioprine), mechanistic target of rapamycin (mTOR) inhibitors such as sirolimus (Rapamune®), Janus kinase inhibitors such as tofacitinib (Xeljanz®), and/or monoclonal antibodies such as basiliximab (Simulect®).

In certain embodiments, the immunosuppressors comprise calcineurin inhibitors, interleukin inhibitors, and/or selective immunosuppressants and TNF alpha inhibitors (such as adalimumab, infliximab, certolizumab pegol, golimumab, and other anti-TNFα neutralizing antibodies or fusion proteins such as etanercept).

In certain embodiments, the laser photocoagulation is conducted in the perimacular region of the eye, at about 1.5-2 disk diameter from the foveal center.

In certain embodiments, the laser photocoagulation comprises a setting of about 50 μm spot size, about 0.1 second's (or 100 ms) duration, and/or about 400˜700 mW intensity.

In certain embodiments, photocoagulation is repeated to produce a break in Bruch's membrane and bubble formation.

In certain embodiments, the laser is argon laser.

In certain embodiments, the NHP is Cynomolgus Monkeys (Macaca fascicularis), stumptailed macaque (Macaca speciosa), rhesus macaques (Macaca mulatta), or African green monkey (Chlorocebus sabaeus).

In certain embodiments, the NHP is Cynomolgus Monkeys (Macaca fascicularis).

Another aspect of the invention provides a method of identifying an inhibitor of wet AMD/CNV development or progression in the NHP model of wet AMD/CNV of the invention, the method comprising contacting the retina of the NHP model of wet AMD/CNV with a candidate inhibitor, and determining the extent said candidate inhibitor inhibits the progression of CNV as compared to a vehicle control, wherein the candidate inhibitor that statistically significantly inhibits CNV progression compared to the vehicle control is selected as the inhibitor of wet AMD/CNV.

In certain embodiments, the candidate inhibitor comes into contact with the retina via subretinal injection.

In certain embodiments, the candidate inhibitor comes into contact with the retina through a canular inserted through a punctuation spot on the eye of the NHP.

In certain embodiments, the candidate inhibitor comes into contact with the retina after first administering one or more immunosuppressors to the NHP.

In certain embodiments, the candidate inhibitor comes into contact with the retina 1, 2, 3, 4, or 5 days after first administering one or more immunosuppressors to the NHP.

In certain embodiments, the candidate inhibitor comes into contact with the retina before (e.g., 3, 4, or 5 weeks before) laser induction of CNV.

In certain embodiments, the candidate inhibitor comprises an AAV viral vector.

It should be understood that any one embodiment described herein, including those described only in the examples or the claims, can be combined with any one or more additional embodiments of the invention unless expressly disclaimed or improper.

### BRIEF DESCRIPTION OF THE DRAWINGS

FIG. 1 is a schematic illustration of treatment of an eye with wetAMD with AAV viral genome expressing hfCas13X.1 (HG-203) decreasing expression of VEGFA.

FIG. 2 is a schematic (not to scale) illustration of an exemplary AAV viral vector genome AAV9-hfCas13X.1-sg^{VEGFA}.

FIG. 3A is a schematic (not to scale) illustration of a control expressing mCherry only.

FIG. 3B is a schematic (not to scale) illustration of a construct expressing hfCas13X.1 and sgVEGFA with mCherry as a reporter.

FIG. 3C is a schematic (not to scale) illustration of a control expressing shRNA with mCherry as a reporter.

FIG. 4 shows a graph of in vitro knockdown of VEGFA by hfCas13X.1 compared to controls expressing CMV-mCherry and shRNA, respectively. Expression of VEGFA were detected by qPCR. N=8/group. *, P<0.05; ***, P<0.001.

FIG. 5 shows a volcano plot showing the statistical significance (P value) versus magnitude of change (fold change) of off-target RNA detection using either AAV9-hfCas13X.1-sg^{VEGFA} (HG-203) versus shRNA against VEGFA.

FIG. 6 shows a graph of total hfCas13X.1 (top ascending curve) and sgVEGFA (bottom ascending curve) expression and VEGFA (descending curve) expression after injection into eyes of C57BL/6 mice. Each group contains 10-20 eyes harvest at various time-points from 8 to 14 weeks post dosing.

FIG. 7 shows a graph of in vivo choroidal neovascularization (CNV) growth inhibition in CNV mouse model. Therapeutic effect of hfCas13X.1-sg^{VEGFA} on a mouse CNV model in comparison to PBS control (negative control) and aflibercept and conbercept (positive controls) is shown. N=25-93/group. *, P<0.05; **, P<0.01; ***, P<0.001.

FIG. 8A shows choroidal neovascularization (CNV) in CNV NHP mouse model after treatment with hfCas13X.1-sg^{VEGFA} (HG-203) or a vehicle control.

FIG. 8B shows a graph of in vivo choroidal neovascularization (CNV) growth inhibition in CNV NHP model. Therapeutic effect of hfCas13X.1-sg^{VEGFA} NHP CNV model measured by CNV area and 5, 6, 8, 10, 13, 18, and 23 weeks post dosing. Vehicle N=29; AAV9 N=23. *, P<0.05; ***, P<0.001. Top curve is vehicle control.

FIG. 8C shows a graph of the ratio of the number of Grade 4 lesions to the Grade 4 lesions at the time of administration of hfCas13X.1-sg^{VEGFA} (HG-203) or vehicle at 5, 6, 8, 10, 13, 18, and 23 weeks after administration. Top curve is vehicle control.

FIG. 9A shows inhibition of CNV in NHP model after administration of hfCas13X.1-sg^{VEGFA} (HG-203) measured by CNV SHRM height. SHRM: subretinal hyper-reflective material; OCT: optical coherence tomography.

FIG. 9B shows a graph of in vivo choroidal neovascularization (CNV) growth inhibition in CNV NHP model. Therapeutic effect of hfCas13X.1-sg^{VEGFA} NHP CNV model measured by CNV SHRM height. Top curve is vehicle control.

FIG. 10 shows a graph of visual function in vivo choroidal neovascularization (CNV) NHP model. Therapeutic effect of hfCas13X.1-sgVEGFA NHP CNV model measured by ERG. Vehicle N=4; AAV9 N=3. *, P<0.05; **, P<0.01; ***, P<0.001.

### DETAILED DESCRIPTION OF THE INVENTION

### 1. Overview

The invention described herein provides reagents and methods for use in treating diseases via an RNA knock-down or editing therapy strategy, e.g., targeting VEGFA to treat wetAMD.

Specifically, the invention described herein provides engineered Cas13 family effector enzymes (referred to herein as Cas13X effector enzymes)—such as the exemplified hfCas13X.1—a class of smaller, safer, and more specific RNA editors, with substantially reduced/eliminated collateral effect. Polynucleotides encoding such engineered Cas13X effector enzymes can be encapsidated in AAV9 serotype capsids for delivery (e.g., subretinal injection) to disease site to knock down or edit target gene (e.g., VEGFA) expression.

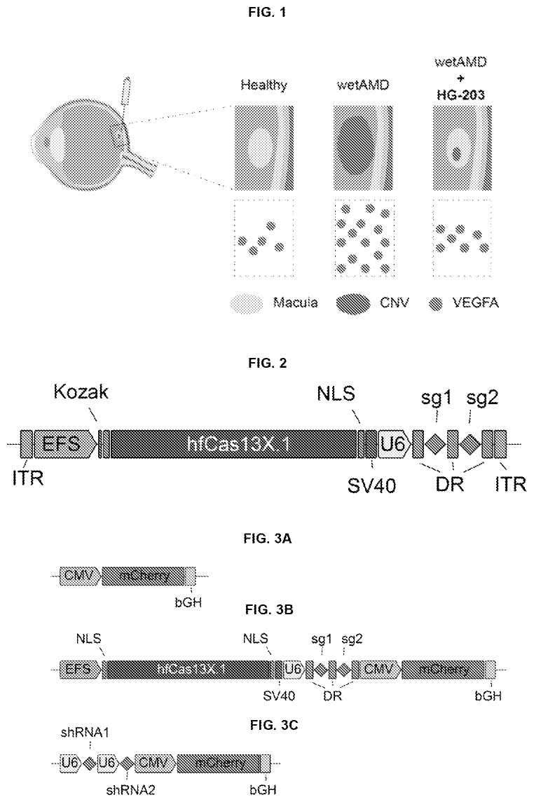

Exemplary constructs of the invention—the subject AA9-EFS-hfCas13X.1-sg^{VEGFA} constructs—such as the one show schematically in FIG. 2, have demonstrated efficacy to treat wetAMD and/or to reduce CNV area (see FIG. 1). The results showed that the subject Cas13X constructs can knockdown expression of a target gene, such as VEGFA, in both cultured cells and experimental animal (e.g., mouse and NHP (non-human primate)) eyes, and the subject AAV9-delivered Cas13X.1 constructs (e.g., AAV9-hfCas13X.1-sg-VEGFA) effectively inhibited CNV growth in both mouse and NHP (non-human primate such as monkey) disease models, thus opening the door for gene therapy to treat wetAMD.

The subject engineered Cas13X effector enzymes, like their respective native forms, display unprecedented sensitivity to recognize specific target RNAs within a heterogeneous population of non-target RNAs—it is believed that the subject engineered Cas13X effector enzymes can detect target RNAs with femtomolar sensitivity.

Wile-type or native Class 2 type VI enzymes or Cas13, from which the subject engineered Cas13X effector enzymes are derived, while offering tremendous opportunity to knock down target gene products (e.g., mRNA) for gene therapy, their use is inherently limited by the co-called collateral activity that poses significant risk of cytotoxicity.

Specifically, in Class 2 type VI systems, a guide sequence non-specific RNA cleavage, referred to as “collateral activity,” is conferred by the higher eukaryotes and prokaryotes nucleotide-binding (HEPN) domain in Cas13 after target RNA binding. Binding of its cognate target ssRNA complementary to the bound crRNA causes substantial conformational changes in Cas13 effector enzyme, leading to the formation of a single, composite catalytic site for guide-sequence independent “collateral” RNA cleavage, thus converting Cas13 into a sequence non-specific ribonuclease. This newly formed highly accessible active site would not only degrade the target RNA in cis if the target RNA is sufficiently long to reach this new active site, but also degrade non-target RNAs in trans based on this promiscuous RNase activity.

Most RNAs appear to be vulnerable to this promiscuous RNase activity of Cas13, and most (if not all) Cas13 effector enzymes possess this collateral endonuclease activity. It has been shown recently that the collateral effects by Cas13-mediated knockdown exist in mammalian cells and animals (manuscript submitted), suggesting that clinical application of Cas13-mediated target RNA knock down will face significant challenge in the presence of collateral effect.

Thus, in order to use the Cas13 enzymes for specifically knocking down a target RNA in gene therapy, it is evident that this guide-sequence non-specific collateral activity must be tightly controlled to prevent unwanted spontaneous cellular toxicity.

The invention described herein provides compositions and methods of use of engineered Class 2 type VI or Cas13 (e.g., Cas13X, such as those based on Cas13e (e.g., Cas13X.1)) to treat eye diseases (e.g., age-related macular degeneration (AMD), e.g., wet AMD).

In one aspect, the invention provides engineered Class 2 type VI or Cas13 (e.g., Cas13e, e.g., Cas13X.1) effector enzymes that largely maintain their sequence-specific endonuclease activity against a target RNA (e.g., vascular endothelial growth factor A (VEGFA), yet with diminished if not eliminated non-guide sequence-specific endonuclease activity against non-target RNAs. Such engineered Cas13X (e.g., Cas13X.1) effector enzymes that substantially lack collateral effect pave the way for using Cas13 in target RNA-knock down-based utility, such as gene therapy. Such engineered Cas13X effector enzymes that substantially lack collateral effect are also useful for RNA-base editing, because a nuclease dead version (or “dCas13”) of such engineered Cas13 also has reduced off-target effect, which is still present in dCas13 without the mutations in the subject engineered Cas13.

While not wishing to be bound by any particular theory, a wild-type Cas13 not only possesses the ability to bind a target RNA through the guide sequence of the crRNA, but also possesses a non-specific RNA binding site for any RNA at the vicinity of the HEPN catalytic domains. Once the target RNA is recognized by the guide sequence, a conformation change of Cas13 activates its catalytic activity, and the target RNA, bound by both the complementary guide sequence and the non-specific RNA binding site, is cleaved. Once activated, Cas13 also non-specifically cleave non-target RNA that does not bind to the guide sequence, partly due to the binding of such non-target RNA to the non-specific RNA binding site on Cas13. Mutations in the non-specific RNA binding motif reduces/eliminates the ability of Cas13 to bind RNA, thus collateral activity against non-target RNA is reduced/eliminated without significantly affecting target RNA cleavage because the target RNA is still bound by the guide sequence.

According to this model, off-target effect in RNA-base editing using a nuclease-deficient (dCas13) version of the engineered Cas13 can also be reduced or eliminated, because the loss of non-specific RNA binding in the engineered dCas13 reduced/eliminates unintended RNA based editing due to the proximity of the RNA base editing domain (e.g., ADAR or CDAR) and an off-target RNA substrate.

More specifically, one aspect of the invention provides an engineered Class 2 type VI Clustered Regularly Interspaced Short Palindromic Repeat (CRISPR)-Cas effector enzyme, such as Cas13 (e.g., Cas13e, e.g., Cas13X.1) wherein the engineered Class 2 type VI Cas effector enzyme: (1) comprises a mutation in a region spatially close to an endonuclease catalytic domain of the corresponding wild-type effector enzyme; (2) substantially preserves guide sequence-specific endonuclease cleavage activity of the wild-type effector enzyme towards a target RNA (such as VEGFA mRNA) complementary to the guide sequence; and, (3) substantially lacks guide sequence-independent collateral endonuclease cleavage activity of the wild-type effector enzyme towards a non-target RNA that is substantially not complement to/does not bind to the guide sequence.

As used herein, “Cas13” is a Class 2 type VI CRISPR-Cas effector enzyme that displays collateral activity as wild-type enzyme upon binding to a cognate target RNA complementary to a guide sequence of its crRNA. The collateral activity of a wild-type Class 2 type VI effector enzyme enables it to cleave RNase or endonuclease activity against a non-target RNA that does not or substantially does not complement with the guide sequence of the crRNA. The wild-type Class 2 type VI effector enzyme may also exhibit one or more of the following characteristics: having one or two conserved HEPN-like RNase domains, such as HEPN domains having the conserved RXXXXH motif (with X being any amino acid), e.g., the RXXXXH motifs described herein below; having a “clenched fist”-like structure when the Class 2 type VI effector enzyme (e.g., Cas13) binds a cognate crRNA; having a bi-lobed structure with a nuclease (NUC) lobe and a crRNA recognition (REC) lobe, optionally, the REC lobe has a variable N-terminal domain (NTD), followed by a helical domain (Helical-1), and/or optionally, the NUC lobe consists of the two HEPN domains (HEPN-1 and HEPN-2) separated by a linker domain (Helical-3), wherein the HEPN-1 domain is optionally split into two subdomains by another helical domain (Helical-2); processes pre-crRNA transcript into crRNA; does not require a trans-activating crRNA (tracrRNA) or other host factors for pre-crRNA processing; and exhibits femtomolar sensitivity to recognize guide sequence-specific target RNAs within a heterogeneous population of non-target RNAs.

In certain embodiments, the Class 2 type VI effector enzyme (e.g., Cas13, e.g., Cas13X.1) has one of the RXXXXN motifs in the HEPN-like domains located at or close to (e.g., within 50-160 residues, or within 50, 60, 70, 80, 90, 100, 110, 120, 130, 140, 150 or 160 residues of) the N-terminus. In certain embodiments, the Class 2 type VI effector enzyme (e.g., Cas13, e.g., Cas13X.1) has one of the RXXXXN motifs in the HEPN-like domains located at or close to (e.g., within 50-160 residues, or within 50, 60, 70, 80, 90, 100, 110, 120, 130, 140, 150 or 160 residues of) the C-terminus. In certain embodiments, the Class 2 type VI effector enzyme (e.g., Cas13, e.g., Cas13X.1) has one of the RXXXXN motifs of the HEPN-like domains located at or close to (e.g., within 50-160 residues, or within 50, 60, 70, 80, 90, 100, 110, 120, 130, 140, 150 or 160 residues of) the N-terminus, while the other of the RXXXXN of the HEPN-like domains is located at or close to (e.g., within 50-160 residues, or within 50, 60, 70, 80, 90, 100, 110, 120, 130, 140, 150 or 160 residues of) the C-terminus. An RXXXXN motif is “at or near” the N- or C-terminus, if either the R or the N residue of the RXXXXN motif is at or near the N- or C-terminus.

Based on biological and cellular experimental data, the engineered Class 2 type VI effector enzyme (e.g., Cas13 particularly Cas13e, e.g., Cas13X.1) effector enzymes have drastically reduced non-sequence-specific endonuclease activity against non-target RNAs, yet simultaneously exhibiting substantially the same if not higher sequence-specific endonuclease activity against a target RNA that substantially complements the guide sequence of the crRNA. The engineered effector enzymes enable high fidelity RNA targeting/editing.

In certain embodiments, the Class 2 type VI effector enzyme is Cas13a, Cas13b, Cas13c, Cas13d, Cas13e (including the engineered variant Cas13X.1), or Cas13f, or an ortholog, paralog, homolog, natural or engineered variant thereof, or functional fragment thereof that substantially maintains the guide sequence-specific endonuclease activity.

In certain embodiments, the variant or functional fragment thereof maintains at least one function of the corresponding wild-type effector enzyme. Such functions include, but are not limited to, the ability to bind a guide RNA/crRNA of the invention (described herein below) to form a complex, the guide sequence-specific RNase activity, and the ability to bind to and cleave a target RNA at a specific site under the guidance of the crRNA that is at least partially complementary to the target RNA.

In some embodiments, the engineered Cas13X protein is an engineered Cas13e protein, e.g., Cas13X.1. In some embodiments, the Cas13e protein is from a species of the genus Planctomycetes.

In certain embodiments, the polynucleotide coding sequence for the wild-type Cas13e.1 protein has the polynucleotide sequence of SEQ ID NO: 1. The encoded wild-type Cas13e.1 protein has the amino acid sequence of SEQ ID NO: 4.

In certain embodiments, the engineered Cas13e.1, e.g., Cas13X.1, protein has an amino acid sequence of SEQ ID NO: 2, e.g., otherwise wild type Cas13e.1 with substitutions at 1-3 residues of Y672, Y676, and I751. In certain embodiments, the substitution substantially reduces or eliminates the collateral activity of the wt Cas13e.1.

For example, the wild-type Cas13e.1 protein sequence SEQ ID NO: 4 may comprises one point mutation at any one of residues Y672, Y676 and I751.

In certain embodiments, the wild-type Cas13e.1 protein sequence SEQ ID NO: 4 may comprises two point mutations at any two of residues Y672, Y676 and I751, such as at Y672 and Y676.

In certain embodiments, the wild-type Cas13e.1 protein sequence SEQ ID NO: 4 may comprises three point mutations at all three residues of Y672, Y676 and I751.

In any of the mutations, the native residue (e.g., Y at 672 and 676, and I at 751) can be substituted by any amino acid other than the native sequence.

In certain embodiments, Y672 can be changed to any of the 19 other amino acids that is not Tyr (Y), such as Ala (A), Cys (C), Asp (D), Glu (E), Phe (F), Gly (G), His (H), Ile (I), Lys (K), Leu (L), Met (M), Asn (N), Pro (P), Gln (Q), Arg (R), Ser (S), Thr (T), Val (V), or Trp (W). In certain embodiments, Y672 can be changed to A, C, D, E, G, H, I, K, L, M, N, Q, R, S, T, or V. In certain embodiments, Y672 can be changed to A, G, I, L, or V. In certain embodiments, Y672 can be changed to A.

In certain embodiments, Y676 can be changed to any of the 19 other amino acids that is not Tyr (Y), such as Ala (A), Cys (C), Asp (D), Glu (E), Phe (F), Gly (G), His (H), Ile (I), Lys (K), Leu (L), Met (M), Asn (N), Pro (P), Gln (Q), Arg (R), Ser (S), Thr (T), Val (V), or Trp (W). In certain embodiments, Y676 can be changed to A, C, D, E, G, H, I, K, L, M, N, P, Q, R, S, T, or V. In certain embodiments, Y676 can be changed to A, G, I, L, or V. In certain embodiments, Y676 can be changed to A.

In certain embodiments, I751 can be changed to any of the 19 other amino acids that is not Ile (I), such as Ala (A), Cys (C), Asp (D), Glu (E), Phe (F), Gly (G), His (H), Lys (K), Leu (L), Met (M), Asn (N), Pro (P), Gln (Q), Arg (R), Ser (S), Thr (T), Val (V), Trp (W), or Tye (Y). In certain embodiments, I751 can be changed to A, C, D, E, G, H, K, L, M, N, P, Q, R, S, T, or V. In certain embodiments, I751 can be changed to A, G, I, L, or V. In certain embodiments, I751 can be changed to A.

In certain embodiments, both Y672 and Y676 are substituted independently by any of the 19 other amino acids that is not Tyr (Y), such as Ala (A), Cys (C), Asp (D), Glu (E), Phe (F), Gly (G), His (H), Ile (I), Lys (K), Leu (L), Met (M), Asn (N), Pro (P), Gln (Q), Arg (R), Ser (S), Thr (T), Val (V), or Tip (W). In certain embodiments, both Y672 and Y676 are substituted independently by A, C, D, E, G, H, I, K, L, M, N, P, Q, R, S, T, or V. In certain embodiments, both Y672 and Y676 are substituted independently by A, G, I, L, or V. In certain embodiments, both Y672 and Y676 are substituted by A.

Thus in SEQ ID NO: 2, (1) Xaa at residue 672 is defined as: any amino acid, except Y when Xaa at 676 is Y and Xaa at 751 is I; (2) Xaa at residue 676 is defined as: any amino acid, except Y when Xaa at 672 is Y and Xaa at 751 is I; and (3) Xaa at residue 751 is defined as: any amino acid, except I when Xaa at 672 and Xaa at 676 are both Y.

In certain embodiments, Xaa at residues 672 and 676 are both Ala (A), and Xaa at residue 751 is Ile (I) (e.g., the engineered Cas13X.1 of SEQ ID NO: 3).

The engineered Cas13e.1, in addition to the mandatory mutations at 1-3 residues of Y672, Y676, and I751, may further comprise one or more additional substitutions, deletions or insertions that collectively result in an engineered Cas13e.1 protein that: (1) has substantially the same (e.g., at least about 80%, 90%, 95%, 99% or more) guide RNA-specific nuclease activity as SEQ ID NO: 4), and (2) has substantially no (e.g., at most 20%, 15%, 10%, 5%) collateral (guide RNA-independent) nuclease activity of SEQ ID NO: 4.

As used herein, “direct repeat sequence” may refer to the DNA coding sequence in the CRISPR locus, or to the RNA encoded by the same in crRNA. Thus when SEQ ID NO: 6 is referred to in the context of an RNA molecule, such as crRNA, each T is understood to represent a U.

In certain embodiments, the engineered Cas13X effector protein of the invention can be: (i) SEQ ID NO: 2, comprising 1-3 substitutions at Y672, Y676, and/or I751; (ii) an ortholog, paralog, homolog of SEQ ID NO: 2 comprising substitutions at Y672, Y676, and/or I751; or (iii) a Class 2 type VI effector enzyme having amino acid sequence identity of at least about 80%, 85%, 90%, 91%, 92%, 93%, 94%, 95%, 96%, 97%, 98%, or 99% compared to any one of SEQ ID NO: 2 comprising substitutions at Y672, Y676, and/or I751.

In certain embodiments, the region spatially close to the endonuclease catalytic domain of the corresponding wild-type Cas13 effector enzyme includes residues within 120, 110, 100, 90, or 80 amino acids from any residues of the endonuclease catalytic domain (e.g., an RXXXXH domain) in the primary sequence of the Cas13.

In certain embodiments, the region spatially close to the endonuclease catalytic domain of the corresponding wild-type Cas13 effector enzyme includes residues more than 100, 110, 120, or 130 residues away from any residues of the endonuclease catalytic domain in the primary sequence of the Cas13, but are spatially within 1-10 or 5 Angstrom of a residue of the endonuclease catalytic domain.

In certain embodiments, the endonuclease catalytic domain is a HEPN domain, optionally a HEPN domain comprising an RXXXXH motif.

In certain embodiments, the RXXXXH motif comprises a R{N/H/K}X₁X₂X₃H sequence.

In certain embodiments, in the R{N/H/K}X₁X₂X₃H sequence, X₁ is R, S, D, E, Q, N, G, or Y; X₂ is I, S, T, V, or L; and X3 is L, F, N, Y, V, I, S, D, E, or A.

In certain embodiments, the RXXXXH motif is an N-terminal RXXXXH motif comprising an RNXXXH sequence, such as an RN{Y/F}{F/Y}SH sequence (SEQ ID NO: 55). In certain embodiments, the N-terminal RXXXXH motif has a RNYFSH sequence (SEQ ID NO: 56). In certain embodiments, the N-terminal RXXXXH motif has a RNFYSH sequence (SEQ ID NO: 57). In certain embodiments, the RXXXXH motif is a C-terminal RXXXXH motif comprising an R{N/A/R}{A/K/S/F}{A/L/F}{F/H/L}H sequence. For example, the C-terminal RXXXXH motif may have a RN(A/K)ALH sequence (SEQ ID NO: 58), or a RAFFHH (SEQ ID NO: 59) or RRAFFH sequence (SEQ ID NO: 60).

In certain embodiments, region comprises, consists essentially of, or consists of residues corresponding to residues between residues 2-187, 227-242, or 634-755 of SEQ ID NO: 2. In certain embodiments, region comprises, consists essentially of, or consists of residues corresponding to residues between residues 35-51, 52-67, 156-171, 666-682, or 712-727 of SEQ ID NO: 2.

In certain embodiments, the mutation comprises, consists essentially of, or consists of substitutions, within a stretch of 15-20 consecutive amino acids within the region, one or more charged or polar residues to a charge neutral short chain aliphatic residue (such as A). For example, in some embodiments, the stretch is about 16 or 17 residues.

In certain embodiments, substantially all, except for up to 1, 2, or 3, charged and polar residues within the stretch are substituted.

In certain embodiments, a total of about 7, 8, 9, or 10 charged and polar residues within the stretch are substituted.

In certain embodiments, the N- and C-terminal 2 residues of the stretch are substituted to amino acids the coding sequences of which contain a restriction enzyme recognition sequence. For example, in some embodiments, the N-terminal two residues may be VF, and the C-terminal 2 residues may be ED, and the restriction enzyme is BpiI. Other suitable RE sites are readily envisioned. The RE sites for the N- and C-terminal ends can be, but need not be identical.

In certain embodiments, the one or more charged or polar residues comprise N, Q, R, K, H, D, E, Y, S, and T residues. In certain embodiments, the one or more charged or polar residues comprise R, K, H, N, Y, and/or Q residues.

In certain embodiments, one or more Y residue(s) within said stretch is substituted. In certain embodiments, said one or more Y residues(s) correspond to Y672 and Y676 of wild-type Cas13e.1 (SEQ ID NO: 4). In certain embodiments, said stretch is residues 35-51, 52-67, 156-171, 666-682, or 712-727 of SEQ ID NO: 4.

In certain embodiments, the mutation leads to reduction or elimination of guide sequence-independent collateral RNase activity.

In certain embodiments, substitution to reduce/eliminate collateral activity includes substitution to charge-neutral short chain aliphatic residues, such as A, I, L, V, or G. In certain embodiments, the charge-neutral short chain aliphatic residue is Ala (A).

In certain embodiments, the mutation to reduce/eliminate collateral activity comprises, consists essentially of, or consists of substitutions within 2, 3, 4, or 5 said stretches of 15-20 consecutive amino acids within the region.

In certain embodiments, the engineered Cas13 preserves at least about 50%, 60%, 70%, 80%, 85%, 90%, 95%, 96%, 97%, 98%, or 99% of the guide sequence-specific endonuclease cleavage activity of the wild-type Cas13 towards the target RNA.

In certain embodiments, the engineered Cas13 lacks at least about 80%, 85%, 90%, 95%, 96%, 97%, 98%, 99%, or 100% of the guide sequence-independent collateral endonuclease cleavage activity of the wild-type Cas13 towards the non-target RNA.

In certain embodiments, the engineered Cas13 preserves at least about 80-90% of the guide sequence-specific endonuclease cleavage activity of the wild-type Cas13 towards the target RNA (e.g., VEGFA), and lacks at least about 95-100% of the guide sequence-independent collateral endonuclease cleavage activity of the wild-type Cas13 towards the non-target RNA.

In certain embodiments, the engineered Cas13 of the invention has an amino acid sequence at least 80%, 85%, 90%, 95%, 96%, 97%, 98%, 99%, 99.1%, 99.2%, 99.3%, 99.4%, 99.5%, 99.6%, 99.7%, 99.8%, or 99.86% identical to any one of SEQ ID NOs: 2 or 3, and comprises the same mutations in SEQ ID NO: 2 or 3 that substantially reduce/eliminate collateral activity. That is, the engineered Cas13 has sequence changes at positions other than/in addition to those corresponding to Y672, Y676, and I751 of Cas13e.1, and such additional sequence changes do not substantially negatively affect the guide sequence-specific endonuclease activity, and/or do not increase the guide sequence-independent collateral effect.

In certain embodiments, the amino acid sequence contains up to 1, 2, 3, 4, or 5 differences (excluding the substitutions at Y672, Y676 and/or I751), without substantially negatively affect the guide sequence-specific endonuclease activity, and/or do not increase the guide sequence-independent collateral effect.

In certain embodiments, the engineered Cas13 of the invention has the amino acid sequence of any one of SEQ ID NOs: 2 or 3. In certain embodiments, the engineered Cas13 of the invention has the amino acid sequence of SEQ ID NO: 3.

In certain embodiments, the engineered Cas13 of the invention has an encoding polynucleotide encoding the amino acid sequence of any one of SEQ ID NOs: 2 or 3. In certain embodiments, the engineered Cas13 of the invention has an encoding polynucleotide encoding the amino acid sequence of SEQ ID NO: 3. In certain embodiments, the encoding polynucleotide has the polynucleotide sequence of SEQ ID NO: 5.

In certain embodiments, the engineered Cas13X of the invention further comprises a nuclear localization signal (NLS) sequence or a nuclear export signal (NES). For example, in certain embodiments, the engineered Cas13X may comprise an N- and/or a C-terminal NLS.

In a related aspect, the invention provides additional derivatives of the subject engineered Cas13, such as those substantially lacking collateral endonuclease activity, such as Cas13e effector proteins based on any one of SEQ ID NOs: 2 or 3, or the above orthologs, homologs, derivatives and functional fragments thereof, which comprises another covalently or non-covalently linked protein or polypeptide or other molecules (such as NLS). Such other proteins/polypeptides/other molecules can be linked through, for example, chemical coupling, gene fusion, or other non-covalent linkage (such as biotin-streptavidin binding). Such derived proteins do not affect the function of the original protein, such as the ability to bind a guide RNA/crRNA of the invention (described herein below) to form a complex, the RNase activity, and the ability to bind to and cleave a target RNA at a specific site, under the guidance of the crRNA that is at least partially complementary to the target RNA. In addition, such derived proteins do retain the characteristics of the subject engineered Cas13 either lacking collateral endonuclease activity.

That is, in certain embodiments, upon binding of the RNP complex of the subject engineered Cas13 (or derivative thereof) to the target RNA, the engineered Cas13 does not exhibit substantial (or detectable) collateral RNase activity.

Such derivation may be used, for example, to add a nuclear localization signal (NLS, such as SV40 large T antigen NLS) to enhance the ability of the subject engineered Cas13, e.g., engineered Cas13e effector proteins, to enter cell nucleus. Such derivation can also be used to add a targeting molecule or moiety to direct the subject Cas13X, e.g., engineered Cas13e effector proteins, to specific cellular or subcellular locations. Such derivation can also be used to add a detectable label to facilitate the detection, monitoring, or purification of the subject Cas13X, e.g., engineered Cas13e effector proteins. Such derivation can further be used to add a deamination enzyme moiety (such as one with adenine or cytosine deamination activity) to facilitate RNA base editing.

The derivation can be through adding any of the additional moieties at the N- or C-terminal of the subject Cas13X effector proteins, or internally (e.g., internal fusion or linkage through side chains of internal amino acids).

In a related aspect, the invention provides conjugates of the subject engineered Cas13. Such conjugated moieties may include, without limitation, localization signals, reporter genes (e.g., GST, HRP, CAT, GFP, HcRed, DsRed, CFP, YFP, BFP), labels (e.g., fluorescent dye such as FITC, or DAPI), NLS, targeting moieties, DNA binding domains (e.g., MBP, Lex A DBD, Gal4 DBD), epitope tags (e.g., His, myc, V5, FLAG, HA, VSV-G, Trx, etc), transcription activation domains (e.g., VP64 or VPR), transcription inhibition domains (e.g., KRAB moiety or SID moiety), nucleases (e.g., FokI), deamination domain (e.g., ADAR1, ADAR2, APOBEC, AID, or TAD), methylase, demethylase, transcription release factor, HDAC, ssRNA cleavage activity, dsRNA cleavage activity, ssDNA cleavage activity, dsDNA cleavage activity, DNA or RNA ligase, any combination thereof, etc.

For example, the conjugate may include one or more NLSs, which can be located at or near N-terminal, C-terminal, internally, or combination thereof. The linkage can be through amino acids (such as D or E, or S or T), amino acid derivatives (such as Ahx, β-Ala, GABA or Ava), or PEG linkage.

In certain embodiments, conjugations do not affect the function of the original engineered protein, such as those either substantially lacking collateral effect, such as the ability to bind a guide RNA/crRNA of the invention (described herein below) to form a complex, and the ability to bind to and cleave a target RNA at a specific site, under the guidance of the crRNA that is at least partially complementary to the target RNA.

In a related aspect, the invention provides fusions of the subject engineered Cas13, such as those substantially lacking collateral endonuclease activity, such as Cas13e effector proteins based on any one of SEQ ID NOs: 2 and 3, or the above orthologs, homologs, derivatives and functional fragments thereof, which fusions are with moieties such as localization signals, reporter genes (e.g., GST, HRP, CAT, GFP, HcRed, DsRed, CFP, YFP, BFP), NLS, protein targeting moieties, DNA binding domains (e.g., MBP, Lex A DBD, Gal4 DBD), epitope tags (e.g., His, myc, V5, FLAG, HA, VSV-G, Trx, etc), transcription activation domains (e.g., VP64 or VPR), transcription inhibition domains (e.g., KRAB moiety or SID moiety), nucleases (e.g., FokI), deamination domain (e.g., ADAR1, ADAR2, APOBEC, AID, or TAD), methylase, demethylase, transcription release factor, HDAC, ssRNA cleavage activity, dsRNA cleavage activity, ssDNA cleavage activity, dsDNA cleavage activity, DNA or RNA ligase, any combination thereof, etc.

For example, the fusion may include one or more NLSs, which can be located at or near N-terminal, C-terminal, internally, or combination thereof. In certain embodiments, conjugations do not affect the function of the original engineered Cas13 protein, such as those substantially lacking collateral activity, such as the ability to bind a guide RNA/crRNA of the invention (described herein below) to form a complex, the RNase activity, and the ability to bind to and cleave a target RNA at a specific site, under the guidance of the crRNA that is at least partially complementary to the target RNA.

In another aspect, the invention provides a polynucleotide encoding the engineered Cas13 (Cas13X) of the invention. The polynucleotide may comprise: (i) a polynucleotide encoding any one of the engineered Cas13 (Cas13X) polypeptide, such as those substantially lacking collateral effect, e.g., those based on Cas13e effector proteins of SEQ ID NOs: 2 and 3, or orthologs, homologs, derivatives, functional fragments, fusions thereof; (ii) a polynucleotide encoding a sgRNA targeting a gene of interest (such as an eye diseases gene of interest, including a wet AMD gene such as VEGFA); or (iii) a polynucleotide comprising (i) and (ii).

In certain embodiments, the polynucleotide of the invention is within an AAV vector genome, flanked by functional AAV (such as AAV2) 5′ and 3′ ITR sequences. In certain embodiments, the AAV vector genome further comprises one or more (e.g., all) of (not necessarily in this order): a promoter (such as an EFS promoter) operably linked to and drives the expression of a coding sequence for the subject Cas13X polypeptide (such as SEQ ID NO: 5) and a polyA signal sequence 3′ thereto, a second promoter operably linked to and drives the expression of one or more DR sequence (such as SEQ ID NO: 6) operably linked to one or more sgRNA coding sequence targeting a target gene (such as VEGFA), and any optional filler, linker, or gap sequences in between the sequence elements.

In certain embodiments, the AAV vector genome further comprises a 3^{rd} transcription unit with a 3^{rd} promoter operably linked to a reporter gene, such as a fluorescent protein reporter (e.g., mCherry or GFP, etc.).

In certain embodiments, the AAV vector genome comprises, consists essentially of, or consists of SEQ ID NO: 17, or a polynucleotide at least about 90%, 91%, 92%, 93%, 94%, 95%, 96%, 97%, 98%, 99%, 99.2%, 99.4%, 99.6%, 99.8%, or 99.9% identical thereto.

In certain embodiments, the polynucleotide of the invention is codon-optimized for expression in a eukaryote, a mammal (such as a human or a non-human mammal), a plant, an insect, a bird, a reptile, a rodent (e.g., mouse, rat), a fish, a worm/nematode, or a yeast.

In a related aspect, the invention provides a polynucleotide having (i) one or more (e.g., 1, 2, 3, 4, 5, 6, 7, 8, 9 or 10) nucleotides additions, deletions, or substitutions compared to the subject polynucleotide described above (e.g., SEQ ID NO: 5 or 17); (ii) at least 50%, 60%, 70%, 80%, 90%, 95%, or 97% sequence identity to the subject polynucleotide described above (e.g., SEQ ID NO: 5 or 17); (iii) hybridize under stringent conditions with the subject polynucleotide described above or any of (i) and (ii); or (iv) is a complement of any of (i)-(iii).

In another related aspect, the invention provides a vector comprising or encompassing any one of the polynucleotide of the invention described herein. The vector can be a cloning vector, viral vector (e.g., AAV, HSV, or baculovius vector) or an expression vector. The vector can be a plasmid, phagemid, or cosmid, just to name a few. In certain embodiments, the vector can be used to express the polynucleotide in a mammalian cell, such as a human cell, any one of the engineered Cas13, such as those substantially lacking collateral activity, e.g., the subject engineered Cas13e or Cas13f effector proteins based on SEQ ID NOs: 2 and 3, or orthologs, homologs, derivatives, functional fragments, fusions thereof; or any of the polynucleotide of the invention; or any of the complex of the invention.

In certain embodiments, the polynucleotide is operably linked to a promoter and optionally an enhancer. For example, in some embodiments, the promoter is a constitutive promoter, an inducible promoter, a ubiquitous promoter, or a tissue specific promoter. In certain embodiments, the vector is a plasmid. In certain embodiments, the vector is a retroviral vector, a phage vector, an adenoviral vector, a herpes simplex viral (HSV) vector, an AAV vector, or a lentiviral vector. In certain embodiments, the AAV vector is a recombinant AAV vector of the serotype AAV1, AAV2, AAV4, AAV5, AAV6, AAV7, AAVrh74, AAV8, AAV9, AAV10, AAV11, AAV 12, or AAV 13. In certain embodiments.

Another aspect of the invention provides a delivery system comprising (1) a delivery vehicle, and (2) the engineered Cas13 of the invention, the polynucleotide of the invention, or the vector of the invention.

In certain embodiments, the delivery vehicle is a nanoparticle, a liposome, an exosome, a microvesicle, or a gene-gun.

A further aspect of the invention provides a cell or a progeny thereof, comprising the engineered Cas13 of the invention, the polynucleotide of the invention, or the vector of the invention. The cell can be a prokaryote such as E. coli, or a cell from a eukaryote such as yeast, insect, plant, animal (e.g., mammal including human and mouse). The cell can be isolated primary cell (such as bone marrow cells for ex vivo therapy), or established cell lines such as tumor cell lines, 293T cells, or stem cells, iPCs, etc.

In certain embodiments, the cell or progeny thereof is a eukaryotic cell (e.g., a non-human mammalian cell, a human cell, or a plant cell) or a prokaryotic cell (e.g., a bacteria cell).

A further aspect of the invention provides a non-human multicellular eukaryote comprising the cell of the invention.

In certain embodiments, the non-human multicellular eukaryote is an animal (e.g., rodent or primate—e.g., NHP) model for a human genetic disorder. In certain embodiments, the NHP is a monkey, such as Cynomolgus Monkeys (Macaca fascicularis).

In another aspect, the invention provides a complex comprising: (i) a protein composition of any one of the subject engineered Cas13X, such as those substantially lacking collateral endonuclease activity, e.g., engineered Cas13e effector protein, or orthologs, homologs, derivatives, conjugates, functional fragments thereof, conjugates thereof, or fusions thereof; and (ii) a polynucleotide composition, comprising an isolated polynucleotide comprising a cognate DR sequence for said engineered Cas13 effector enzyme, and a spacer/guide sequence complementary to at least a portion of a target RNA (e.g., a VEGFA target mRNA).

In certain embodiments, the DR sequence is at the 3′ end of the spacer sequence.

In certain embodiments, the DR sequence is at the 5′ end of the spacer sequence.

In some embodiments, the polynucleotide composition is the guide RNA/crRNA of the subject engineered Cas13, such as those substantially lacking collateral activity, e.g., engineered Cas13e system, which does not include a tracrRNA.

In certain embodiments, for use with the subject engineered Cas13, such as those substantially lacking collateral activity, e.g., the subject engineered Cas13e effector proteins, homologs, orthologs, derivatives, fusions, conjugates, or functional fragments thereof having guide sequence-specific RNase activity, the spacer sequence is at least about 10 nucleotides, or between 10-60, 15-50, 20-50, 25-40, 25-50, or 19-50 nucleotides.

In a related aspect, the invention provides a eukaryotic cell comprising a subject complex comprising a subject engineered Cas13, said complex comprising: (1) an RNA guide sequence comprising a spacer sequence capable of hybridizing to a target RNA (e.g., a VEGFA target mRNA), and a direct repeat (DR) sequence 5′ or 3′ to the spacer sequence; and, (2) a subject engineered Cas13, such as those substantially lacking collateral activity, such as a subject engineered Cas13e effector enzyme (such as SEQ ID NOs: 2 and 3) based on a wild-type having an amino acid sequence of SEQ ID NO: 4, or a derivative or functional fragment of said Cas; wherein the Cas, the derivative, and the functional fragment of said Cas, are capable of (i) binding to the RNA guide sequence and (ii) targeting the target RNA (e.g., a VEGFA target mRNA).

In another aspect, the invention provides a composition comprising: (i) a first (protein) composition selected from any one of the engineered Cas13, such as those substantially lacking collateral activity, e.g., engineered Cas13e effector proteins based on SEQ ID NOs: 2 and 3, or orthologs, homologs, derivatives, conjugates, functional fragments, fusions thereof; and (ii) a second (nucleotide) composition comprising an RNA encompassing a guide RNA/crRNA, particularly a spacer sequence, or a coding sequence for the same. The guide RNA may comprise a DR sequence, and a spacer sequence which can complement or hybridize with a target RNA (e.g., a VEGFA target mRNA). The guide RNA can form a complex with the first (protein) composition of (i). In some embodiment, the DR sequence can be the polynucleotide of the invention (such as SEQ ID NO: 6). In some embodiment, the DR sequence can be at the 5- or 3′-end of the guide RNA. In some embodiments, the composition (such as (i) and/or (ii)) is non-naturally occurring or modified from a naturally occurring composition. In some embodiments, the target sequence is an RNA transcript of the VEGFA gene (such as human VEGFA). The target RNA may be present inside a cell, such as in the cytosol or inside an organelle. In some embodiments, the protein composition may have an NLS that can be located at its N- or C-terminal, or internally.

In another aspect, the invention provides a composition comprising one or more vectors of the invention, said one or more vectors comprise: (i) a first polynucleotide (such as SEQ ID NO: 5) that encodes any one of the engineered Cas13, such as those substantially lacking collateral activity, such as a subject engineered Cas13e effector proteins based on SEQ ID NOs: 2 and 3, or orthologs, homologs, derivatives, functional fragments, fusions thereof; optionally operably linked to a first regulatory element (such as an EF1a promoter or a functional fragment thereof, e.g., EFS promoter); and (ii) a second polynucleotide that encodes a guide RNA of the invention; optionally operably linked to a second regulatory element (such as a U6 promoter). The first and the second polynucleotides can be on different vectors, or on the same vector (e.g., on the same AAV vector genome). The guide RNA can form a complex with the protein product encoded by the first polynucleotide, and comprises a DR sequence (such as any one of the 4th aspect) and a spacer sequence that can bind to/complement with a target RNA (such as an mRNA of VEGFA). In some embodiments, the first regulatory element is a promoter, such as a constitutive promoter or an inducible promoter. In some embodiments, the second regulatory element is a promoter, such as a constitutive promoter (such as a Pol III promoter like U6) or an inducible promoter. In some embodiments, the target sequence is an RNA from a prokaryote or a eukaryote, such as a mammalian (e.g., human) VEGFA mRNA. The target RNA may be present inside a cell, such as in the cytosol or inside an organelle. In some embodiments, the protein composition may have an NLS that can be located at its N- and/or C-terminal, or internally.

In some embodiments, the vector is a plasmid. In some embodiment, the vector is a viral vector based on a retrovirus, a replication incompetent retrovirus, adenovirus, replication incompetent adenovirus, or AAV. In some embodiments, the vector can self-replicate in a host cell (e.g., having a bacterial replication origin sequence). In some embodiments, the vector can integrate into a host genome and be replicated therewith. In some embodiment, the vector is a cloning vector. In some embodiment, the vector is an expression vector.

The invention further provides a delivery composition for delivering any of the engineered Cas13, such as those substantially lacking collateral activity, e.g., a subject engineered Cas13e effector proteins based on SEQ ID NOs: 2 and 3, or orthologs, homologs, derivatives, conjugates, functional fragments, fusions thereof of the invention; the polynucleotide of the invention; the complex of the invention; the vector of the invention; the cell of the invention, and the composition of the invention. The delivery can be through any one known in the art, such as transfection, lipofection, electroporation, gene gun, microinjection, sonication, calcium phosphate transfection, cation transfection, viral vector delivery, etc., using vehicles such as liposome(s), nanoparticle(s), exosome(s), microvesicle(s), a gene-gun or one or more viral vector(s).

The invention further provides a kit comprising any one or more of the following: any of the engineered Cas13, such as those substantially lacking collateral activity, e.g., a subject engineered Cas13e or Cas13f effector proteins based on SEQ ID NOs: 2 and 3, or orthologs, homologs, derivatives, conjugates, functional fragments, fusions thereof of the invention; the polynucleotide of the invention; the complex of the invention; the vector of the invention; the cell of the invention, and the composition of the invention. In some embodiments, the kit may further comprise an instruction for how to use the kit components, and/or how to obtain additional components from 3^{rd} party for use with the kit components. Any component of the kit can be stored in any suitable container.

With the inventions generally described herein above, more detailed descriptions for the various aspects of the invention are provided in separate sections below. However, it should be understood that, for simplicity and to reduce redundancy, certain embodiments of the invention are only described under one section or only described in the claims or examples. Thus it should also be understood that any one embodiment of the invention, including those described only under one aspect, section, or only in the claims or examples, can be combined with any other embodiment of the invention, unless specifically disclaimed or the combination is improper.

### 2. Representative Engineered Class 2 Type VI Cas and Derivatives Thereof

One aspect of the invention provides engineered Cas13, such as those substantially lacking collateral activity.

In certain embodiments, the Cas13 effector enzyme is a Class 2, type VI effector enzyme having two strictly conserved RX4-6H (RXXXXH)-like motifs, characteristic of Higher Eukaryotes and Prokaryotes Nucleotide-binding (HEPN) domains. In certain embodiments, the CRISPR Class 2, type VI effectors that contain two HEPN domains, for example, CRISPR Cas13e (including engineered variant Cas13X.1).

HEPN domains have been shown to be RNase domains and confer the ability to bind to and cleave target RNA molecule. The target RNA may be any suitable form of RNA, including but not limited to mRNA, tRNA, ribosomal RNA, non-coding RNA, lncRNA (long non-coding RNA), and nuclear RNA. For example, in some embodiments, the engineered Cas13 proteins recognize and cleave RNA targets located on the coding strand of open reading frames (ORFs).

In one embodiment, the Class 2 type VI Cas13 effector enzyme is of the subtype Type VI-E or Cas13e (such as SEQ ID NOs: 2 and 3). Direct comparison of the wild-type Type VI-E CRISPR-Cas effector proteins with the effector of these other systems shows that Type VI-E CRISPR-Cas effector proteins are significantly smaller (e.g., about 20% fewer amino acids) than even the smallest previously identified Type VI-D/Cas13d effectors, and have less than 30% sequence similarity in one to one sequence alignments to other previously described effector proteins, including the phylogenetically closest relatives Cas13b.

Class 2, subtype VI-E effector, like other Cas13 proteins, can be used in a variety of applications, and are particularly suitable for therapeutic applications since they are significantly smaller than other effectors (e.g., CRISPR Cas13a, Cas13b, Cas13c, and Cas13d/CasRx effectors) which allows for the packaging of the nucleic acids encoding the effectors and their guide RNA coding sequences into delivery systems having size limitations, such as the AAV vectors. Further, the lack of detectable collateral/non-specific RNase activity of the subject engineered Cas13, upon activation of the guide sequence-specific RNase activity, makes these engineered Cas13 effectors less prong to (if not immune from) potentially dangerous generalized off-target RNA digestion in target cells that are desirably not destroyed.

Exemplary Type VI-E CRISPR-Cas effector proteins ae provided in the table below.

| | |
|---|---|
| Cas13X.1 | MAQVSKQTSKKRELSIDEYQGARKWCFTIAFNKALVNRDKNDGLFVESL |
| (comprising | LRHEKYSKHDWYDEDTRALIKCSTQAANAKAEAL [symbol] HYRHSPGCL |
| substitutions | TFTAEDELRTIMERAYERAIFECRRRETEVIIEFPSLFEGDRITTAGVVF |
| at Y672, | FVSFFVERRVLDRLYGAVSGLKKNEGQYKLTRKALSMYCLKDSRFTKAWD |
| Y676, and/or | KRVLLFRDILAQLGRIPAEAYEYYHGEQGDKKRANDNEGTNPKRHKDK |
| 1751 | FIEFALHYLEAQHSEICFGRRHIVREEAGAGDEHKKHRTKGKVVVDFSK |
| (SEQ ID | KDEDQSYYISKNNVIVRIDKNAGPRSYRMGLNELKYLVLLSLQGKGDDA |
| NO: 2) | IAKLYRYRQHVENILDVVKVTDKDNHVFLPRFVLEQHGIGRKAFKQRID |
| | GRVKHVRGVWEKKKAATNEMTLHEKARDILQYVNENCTRSFNPGEYN |
| | RLLVCLVGKDVENFQAGLKRLQLAERIDGRVYSIFAQTSTINEMHQVVC |
| | DQILNRLCRIGDQKLYDYVGLGKKDEIDYKQKVAWFKEHISIRRGFLRK |
| | KFWYDSKKGFAKLVEEHLESGGGQRDVGLDKKYYHIDAIGRFEGANPA |
| | LYETLARDRLCLMMAQYFLGSVRKELGNKIVWSNDSIELPVEGSVGNEK |
| | SIVFSVSDYGKLYVLDDAEFLGRICEYFMPHEKGKIR(Xaa)HTV(Xaa) |
| | EKGFRAYNDLQKKCVEAVLAFEEKVVKAKKMSEKEGAHYIDFREILAQT |
| | MCKEAEKTAVNKV [symbol] HHLKFV(Xaa)DEFGLFSDVMKKYGIEKEWK |
| | FPVK (SEQ ID NO: 2) |
| Cas13X.1 | MAQVSKQTSKKRELSIDEYQGARKWCFTIAFNKALVNRDKNDGLFVESL |
| (SEQ ID | LRHEKYSKHDWYDEDTRALIKCSTQAANAKAEAL [symbol] YRHSPGCL |
| NO: 3) | TFTAEDELRTIMERAYERAIFECRRRETEVIIEFPSLFEGDRITTAGVVF |
| Y672A, | FVSFFVERRVLDRLYGAVSGLKKNEGQYKLTRKALSMYCLKDSRFTKAWD |
| Y676A in | KRVLLFRDILAQLGRIPAEAYEYYHGEQGDKKRANDNEGTNPKRHKDK |
| [symbol] | FIEFALHYLEAQHSEICFGRRHIVREEAGAGDEHKKHRTKGKVVVDFSK |
| | KDEDQSYYISKNNVIVRIDKNAGPRSYRMGLNELKYLVLLSLQGKGDDA |
| | IAKLYRYRQHVENILDVVKVTDKDNHVFLPRFVLEQHGIGRKAFKQRID |
| | GRVKHVRGVWEKKKAATNEMTLHEKARDILQYVNENCTRSFNPGEYN |
| | RLLVCLVGKDVENFQAGLKRLQLAERIDGRVYSIFAQTSTINEMHQVVC |
| | DQILNRLCRIGDQKLYDYVGLGKKDEIDYKQKVAWFKEHISIRRGFLRK |
| | KFWYDSKKGFAKLVEEHLESGGGQRDVGLDKKYYHIDAIGRFEGANPA |
| | LYETLARDRLCLMMAQYFLGSVRKELGNKIVWSNDSIELPVEGSVGNEK |
| | SIVFSVSDYGKLYVLDDAEFLGRICEYFMPHEKGKIRAHTVAEKGFRAYN |
| | DLQKKCVEAVLAFEEKVVKAKKMSEKEGAHYIDFREILAQTMCKEAEK |
| | TAVNKV [symbol] HHLKFVIDEFGLFSDVMKKYGIEKEWKFPVK |
| | (SEQ ID NO: 3) |

In the sequences above, the two RX4-6H (RXXXXH) motifs in each effector are double-underlined. In Cas13e.1, the C-terminal motif may have two possibilities due to the RR and HH sequences flanking the motif. Mutations at one or both such domains may create an RNase dead version (or “dCas”) of the Cas13e and Cas13f effector proteins, homologs, orthologs, fusions, conjugates, derivatives, or functional fragments thereof, while substantially maintaining their ability to bind the guide RNA and the target RNA complementary to the guide RNA.

The corresponding DR coding sequences for the Cas effectors are listed below:

| | |
|---|---|
| Cas13e | GCTGGAGCAGCCCCCGATTTGTGGGGTGATTACAGC |
| DR | (SEQ ID NO: 6) |

In some embodiments, a subject engineered Cas13 effector enzyme, such as those substantially lacking collateral activity is based on a “derivative” of a wild-type Type VI-E CRISPR-Cas effector proteins, said derivative having an amino acid sequence with at least about 80% sequence identity to the amino acid sequence of any one of SEQ ID NOs: 2 and 3 above (e.g., 81%, 82%, 83%, 84%, 85%, 86%, 87% 88%, 89%, 90%, 91%, 92%, 93%, 94%, 95%, 96%, 97%, 98%, 99%, or 100%), including having the same mutations at Y672/Y676/I751. Such derivative Cas effectors sharing significant protein sequence identity to any one of SEQ ID NOs: 2 and 3 have retained at least one of the functions of the Cas of SEQ ID NOs: 2 and 3 (see below), such as the ability to bind to and form a complex with a crRNA comprising at least one of the DR sequences of SEQ ID NO: 6. For example, a Cas3e.1 derivative may share 85% amino acid sequence identity to SEQ ID NO: 2 and 3, respectively, and retains the ability to bind to and form a complex with a crRNA having a DR sequence of SEQ ID NO: 6, respectively.

In some embodiments, the derivative comprises conserved amino acid residue substitutions. In some embodiments, the derivative comprises only conserved amino acid residue substitutions (i.e., all amino acid substitutions in the derivative are conserved substitutions, and there is no substitution that is not conserved).

In some embodiments, the derivative comprises no more than 1, 2, 3, 4, 5, 6, 7, 8, 9, or 10 amino acid additional insertions or deletions compared to SEQ ID NOs: 2 or 3. The insertion and/or deletion maybe clustered together, or separated throughout the entire length of the sequences, so long as at least one of the functions of the SEQ ID NOs: 2 or 3 sequence is preserved. Such functions may include the ability to bind the guide/crRNA, the RNase activity, the ability to bind to and/or cleave the target RNA complementary to the guide/crRNA. In some embodiments, the insertions and/or deletions are not present in the RXXXXH motifs, or within 5, 10, 15, or 20 residues from the RXXXXH motifs.

In some embodiments, the derivative has retained the ability to bind guide RNA/crRNA.

In some embodiments, the derivative has retained the guide/crRNA-activated RNase activity.

In some embodiments, the derivative has retained the ability to bind target RNA and/or cleave the target RNA in the presence of the bound guide/crRNA that is complementary in sequence to at least a portion of the target RNA.

In other embodiments, the derivative has completely or partially lost the guide/crRNA-activated RNase activity, due to, for example, mutations in one or more catalytic residues of the RNA-guided RNase. Such derivatives are sometimes referred to as dCas, such as dCas13X.1.

In certain embodiments, the effector protein as described herein is a “dead” effector protein, such as a dead Cas13e effector protein (i.e. dCas13e). In certain embodiments, the effector protein has one or more mutations in HEPN domain 1 (N-terminal). In certain embodiments, the effector protein has one or more mutations in HEPN domain 2 (C-terminal). In certain embodiments, the effector protein has one or more mutations in HEPN domain 1 and HEPN domain 2.

The inactivated Cas or derivative or functional fragment thereof can be fused or associated with one or more heterologous/functional domains (e.g., via fusion protein, linker peptides, “GS” linkers, etc.). These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, base-editing activity, and switch activity (e.g., light inducible). In some embodiments, the functional domains are Krüppel associated box (KRAB), SID (e.g. SID4X), VP64, VPR, VP16, Fok1, P65, HSF1, MyoD1, Adenosine Deaminase Acting on RNA such as ADAR1, ADAR2, APOBEC, cytidine deaminase (AID), TAD, mini-SOG, APEX, and biotin-APEX.

In some embodiments, the functional domain is a base editing domain, e.g., ADAR1 (including wild-type or ADAR2DD version thereof, with or without the E1008Q and/or the E488Q mutation(s)), ADAR2 (including wild-type or ADAR2DD version thereof, with or without the E1008Q and/or the E488Q mutation(s)), APOBEC, or AID.

In some embodiments, the functional domain may comprise one or more nuclear localization signal (NLS) domains. The one or more heterologous functional domains may comprise at least two or more NLS domains. The one or more NLS domain(s) may be positioned at or near or in proximity to a terminus of the effector protein (e.g., Cas13e/effector proteins) and if two or more NLSs, each of the two may be positioned at or near or in proximity to a terminus of the effector protein (e.g., Cas13e effector proteins).

In some embodiments, at least one or more heterologous functional domains may be at or near the amino-terminus of the effector protein and/or wherein at least one or more heterologous functional domains is at or near the carboxy-terminus of the effector protein. The one or more heterologous functional domains may be fused to the effector protein. The one or more heterologous functional domains may be tethered to the effector protein. The one or more heterologous functional domains may be linked to the effector protein by a linker moiety.

In some embodiments, multiple (e.g., two, three, four, five, six, seven, eight, or more) identical or different functional domains are present.

In some embodiments, the functional domain (e.g., a base editing domain) is further fused to an RNA-binding domain (e.g., MS2).

In some embodiments, the functional domain is associated to or fused via a linker sequence (e.g., a flexible linker sequence or a rigid linker sequence). Exemplary linker sequences and functional domain sequences are provided in table below.

Amino Acid Sequences of Motifs and Functional Domains in
Engineered Variants of Type VI-E CRISPR Cas Effectors

| | |
|---|---|
| Linker 1 | GS |
| Linker 2 | GSGGGGS (SEQ ID NO: 61) |
| Linker 3 | GGGGSGGGGSGGGGS (SEQ ID NO: 62) |
| ADARID | SLGTGNRCVKGDSLSLKGETVNDCHAEIISRRGFIRFLYSELMKYNSQTAKD |
| D-WT | SIFEPAKGGEKLQIKKTVSFHLYISTAPCGDGALFDKSCSDRAMESTESRHY |
| | PVFENPKQGKLRTKVENGEGTIPVESSDIVPTWDGIRLGERLRTMSCSDKIL |
| | RWNVLGLQGALLTHFLQPIYLKSVTLGYLFSQGHLTRAICCRVTRDGSAFE |
| | DGLRHPFIVNHPKVGRVSIYDSKRQSGKTKETSVNWCLADGYDLEILDGTR |
| | GTVDGPRNELSRVSKKNIFLLFKKLCSFRYRRDLLRLSYGEAKKAARDYET |
| | AKNYFKKGLKDMGYGNWISKPQEEKNF (SEQ ID NO: 63) |
| AD ARID | SLGTGNRCVKGDSLSLKGETVNDCHAEIISRRGFIRFLYSELMKYNSQTAKD |
| D-E1008Q | SIFEPAKGGEKLQIKKTVSFHLYISTAPCGDGALFDKSCSDRAMESTESRHY |
| | PVFENPKQGKLRTKVENGQGTIPVESSDIVPTWDGIRLGERLRTMSCSDKIL |
| | RWNVLGLQGALLTHFLQPIYLKSVTLGYLFSQGHLTRAICCRVTRDGSAFE |
| | DGLRHPFIVNHPKVGRVSIYDSKRQSGKTKETSVNWCLADGYDLEILDGTR |
| | GTVDGPRNELSRVSKKNIFLLFKKLCSFRYRRDLLRLSYGEAKKAARDYET |
| | AKNYFKKGLKDMGYGNWISKPQEEKNF (SEQ ID NO: 64) |
| ADAR2D | QLHLPQVLADAVSRLVLGKFGDLTDNFSSPHARRKVLAGVVMTTGTDVKDA |
| D-WT | KVISVSTGTKCINGEYMSDRGLALNDCHAEIISRRSLLRFLYTQLELYLNN |
| | KDDQKRSIFQKSERGGFRLKENVQFHLYISTSPCGDARIFSPHEPILEEPA |
| | DRHPNRKARGQLRTKIESGEGTIPVRSNASIQTWDGVLQGERLLTMSCSDK |
| | IARWNVVGIQGSLLSIFVEPIYFSSIILGSLYHGDHLSRAMYQRISNIEDL |
| | PPLYTLNKPLLSGISNAEARQPGKAPNFSVNWTVGDSAIEVINATTGKDEL |
| | GRASRLCKHALYCRWMRVHGKVPSHLLRSKITKPNVYHESKLAAKEYQAAK |
| | ARLFTAFIKAGLGAWVEKPTEQDQFSLT (SEQ ID NO: 65) |
| ADAR2D | QLHLPQVLADAVSRLVLGKFGDLTDNFSSPHARRKVLAGVVMTTGTDVKDA |
| D-E488Q | KVISVSTGTKCINGEYMSDRGLALNDCHAEIISRRSLLRFLYTQLELYLNN |
| | KDDQKRSIFQKSERGGFRLKENVQFHLYISTSPCGDARIFSPHEPILEEPA |
| | DRHPNRKARGQLRTKIESGQGTIPVRSNASIQTWDGVLQGERLLTMSCSDK |
| | IARWNVVGIQGSLLSIFVEPIYFSSIILGSLYHGDHLSRAMYQRISNIEDL |
| | PPLYTLNKPLLSGISNAEARQPGKAPNFSVNWTVGDSAIEVINATTGKDEL |
| | GRASRLCKHALYCRWMRVHGKVPSHLLRSKITKPNVYHESKLAAKEYQAAK |
| | ARLFTAFIKAGLGAWVEKPTEQDQFSLT (SEQ ID NO: 66) |
| AID- | MDSLLMNRRKFLYQFKNVRWAKGRRETYLCYVVKRRDSATSFSLDFGYLRN |
| APOBEC | KNGCHVELLFLRYISDWDLDPGRCYRVTWFTSWSPCYDCARHVADFLRGNP |
| 1 | NLSLRIFTARLYFCEDRKAEPEGLRRLHRAGVQIAIMTFKDYFYCWNTFVE |
| | NHERTFKAWEGLHENSVRLSRQLRRILLPLYEVDDLRDAFRTLGL |
| | (SEQ ID NO: 67) |
| Lamprey | MTDAEYVRIHEKLDIYTFKKQFFNNKKSVSHRCYVLFELKRRGERRACFWG |
| AID- | YAVNKPQSGTERGIHAEIFSIRKVEEYLRDNPGQFTINWYSSWSPCADCAE |
| APOBEC | KILEWYNQELRGNGHTLKIWACKLYYEKNARNQIGLWNLRDNGVGLNVMVS |
| 1 | EHYQCCRKIFIQSSHNQLNENRWLEKTLKRAEKRRSELSIMIQVKILHTTK |
| | SPAV (SEQ ID NO: 68) |
| APOBEC | MSSETGPVAVDPTLRRRIEPHEFEVFFDPRELRKETCLLYEINWGGRHSIW |
| 1_BE1 | RHTSQNTNKHVEVNFIEKFTTERYFCPNTRCSITWFLSWSPCGECSRAITE |
| | FLSRYPHVTLFIYIARLYHHADPRNRQGLRDLISSGVTIQIMTEQESGYCW |
| | RNFVNYSPSNEAHWPRYPHLWVRLYVLELYCIILGLPPCLNILRRKQPQLT |
| | FFTIALQSCHYQRLPPHILWATGLK (SEQ ID NO: 69) |

In some embodiments, instead of using full-length wild-type or derivative Type VI-E and VI-F Cas effectors, “functional fragments” thereof can be used.

A “functional fragment,” as used herein, refers to a fragment of a functional Cas13 protein such as any one of SEQ ID NOs: 2 and 3, or a derivative thereof, that has less-than full-length sequence. The deleted residues in the functional fragment can be at the N-terminus, the C-terminus, and/or internally. The functional fragment retains at least one function of the original functional VI-E or VI-F Cas, or at least one function of its derivative. Thus a functional fragment is defined specifically with respect to the function at issue. In certain embodiments, the engineered Cas13 of the invention including a functional fragment of an engineered Cas13 that substantially retains the corresponding original Cas13's (e.g., Cas13e.1) guide sequence-dependent RNase activity, but substantially lacks collateral activity.

In some embodiments, compared to full-length wild-type sequences, the engineered Class 2 type VI effector proteins or derivatives thereof or functional fragments thereof lacks about 30, 60, 90, 120, 150, or about 180 residues from the N-terminus.

In some embodiments, compared to full-length wild-type sequences, the engineered Class 2 type VI effector proteins or derivatives thereof or functional fragments thereof lacks about 30, 60, 90, 120, or about 150 residues from the C-terminus.

In some embodiments, compared to full-length wild-type sequences, the engineered Class 2 type VI effector proteins or derivatives thereof or functional fragments thereof lacks about 30, 60, 90, 120, 150, or about 180 residues from the N-terminus, and lacks about 30, 60, 90, 120, or about 150 residues from the C-terminus.

In some embodiments, the engineered Class 2 Type VI Cas13 effector proteins or derivatives thereof or functional fragments thereof have RNase activity, e.g., guide/crRNA-activated specific RNase activity.

In some embodiments, the engineered Class 2 Type VI Cas13 effector proteins or derivatives thereof or functional fragments thereof have no substantial/detectable collateral RNase activity.

The present disclosure also provides a split version of the engineered Class 2 type VI Cas13 effector enzyme described herein (e.g., a Type VI-E or VI-F CRISPR-Cas effector protein). The split version of the engineered Cas13 may be advantageous for delivery. In some embodiments, the engineered Cas13 is split into two parts of the enzyme, which together substantially comprise a functioning engineered Class 2 type VI Cas13.

The split can be done in a way that the catalytic domain(s) are unaffected. The CRISPR-associated protein may function as a nuclease or may be an inactivated enzyme, which is essentially a RNA-binding protein with very little or no catalytic activity (e.g., due to mutation(s) in its catalytic domains). Split enzymes are described, e.g., in Wright et al., “Rational design of a split-Cas9 enzyme complex,” Proc. Nat'l. Acad. Sci. 112(10): 2984-2989, 2015, which is incorporated herein by reference in its entirety.

For example, in some embodiments, the nuclease lobe and α-helical lobe are expressed as separate polypeptides. Although the lobes do not interact on their own, the crRNA recruits them into a ternary complex that recapitulates the activity of full-length CRISPR-associated proteins and catalyzes site-specific cleavage. The use of a modified crRNA abrogates split-enzyme activity by preventing dimerization, allowing for the development of an inducible dimerization system.

In some embodiments, the split CRISPR-associated protein can be fused to a dimerization partner, e.g., by employing rapamycin sensitive dimerization domains. This allows the generation of a chemically inducible CRISPR-associated protein for temporal control of the activity of the protein. The CRISPR-associated protein can thus be rendered chemically inducible by being split into two fragments and rapamycin-sensitive dimerization domains can be used for controlled re-assembly of the protein.

The split point is typically designed in silico and cloned into the constructs. During this process, mutations can be introduced to the split CRISPR-associated protein and non-functional domains can be removed.

In some embodiments, the two parts or fragments of the split CRISPR-associated protein (i.e., the N-terminal and C-terminal fragments), can form a full CRISPR-associated protein, comprising, e.g., at least 70%, at least 80%, at least 90%, at least 95%, or at least 99% of the sequence of the wild-type CRISPR-associated protein.

The CRISPR-associated proteins described herein (e.g., a Type VI-E or VI-F CRISPR-Cas effector protein) can be designed to be self-activating or self-inactivating. For example, the target sequence can be introduced into the coding construct of the CRISPR-associated protein. Thus, the CRISPR-associated protein can cleave the target sequence, as well as the construct encoding the protein thereby self-inactivating their expression. Methods of constructing a self-inactivating CRISPR system are described, e.g., in Epstein and Schaffer, Mol. Ther. 24: S50, 2016, which is incorporated herein by reference in its entirety.

In some other embodiments, an additional crRNA, expressed under the control of a weak promoter (e.g., 7SK promoter), can target the nucleic acid sequence encoding the CRISPR-associated protein to prevent and/or block its expression (e.g., by preventing the transcription and/or translation of the nucleic acid). The transfection of cells with vectors expressing the CRISPR-associated protein, the crRNAs, and crRNAs that target the nucleic acid encoding the CRISPR-associated protein can lead to efficient disruption of the nucleic acid encoding the CRISPR-associated protein and decrease the levels of CRISPR-associated protein, thereby limiting its activity.

In some embodiments, the activity of the CRISPR-associated protein can be modulated through endogenous RNA signatures (e.g., miRNA) in mammalian cells. A CRISPR-associated protein switch can be made by using a miRNA-complementary sequence in the 5′-UTR of mRNA encoding the CRISPR-associated protein. The switches selectively and efficiently respond to miRNA in the target cells. Thus, the switches can differentially control the Cas activity by sensing endogenous miRNA activities within a heterogeneous cell population. Therefore, the switch systems can provide a framework for cell-type selective activity and cell engineering based on intracellular miRNA information (see, e.g., Hirosawa et al., Nucl. Acids Res. 45(13): e118, 2017).

The engineered Class 2 type VI Cas13 effectors, such as those substantially lacking collateral activity (e.g., engineered Type VI-E and VI-F CRISPR-Cas effector proteins) can be inducibly expressed, e.g., their expression can be light-induced or chemically-induced. This mechanism allows for activation of the functional domain in the CRISPR-associated proteins. Light inducibility can be achieved by various methods known in the art, e.g., by designing a fusion complex wherein CRY2 PHR/CIBN pairing is used in split CRISPR-associated proteins (see, e.g., Konermann et al., “Optical control of mammalian endogenous transcription and epigenetic states,” Nature 500:7463, 2013.

Chemical inducibility can be achieved, e.g., by designing a fusion complex wherein FKBP/FRB (FK506 binding protein/FKBP rapamycin binding domain) pairing is used in split CRISPR-associated proteins. Rapamycin is required for forming the fusion complex, thereby activating the CRISPR-associated proteins (see, e.g., Zetsche et al., “A split-Cas9 architecture for inducible genome editing and transcription modulation,” Nature Biotech. 33:2:139-42, 2015).

Furthermore, expression of the engineered Class 2 type VI Cas13 effectors, such as those substantially lacking collateral activity can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the RNA targeting effector protein can be modulated via a riboswitch, which can sense a small molecule like tetracycline (see, e.g., Goldfless et al., “Direct and specific chemical control of eukaryotic translation with a synthetic RNA-protein interaction,” Nucl. Acids Res. 40:9: e64-e64, 2012).

Various embodiments of inducible CRISPR-associated proteins and inducible CRISPR systems are described, e.g., in U.S. Pat. No. 8,871,445, US Publication No. 2016/0208243, and International Publication No. WO 2016/205764, each of which is incorporated herein by reference in its entirety.

In some embodiments, the engineered Class 2 type VI Cas13 effectors, such as those substantially lacking collateral activity include at least one (e.g., 1, 2, 3, 4, 5, 6, 7, 8, 9, or 10) Nuclear Localization Signal (NLS) attached to the N-terminal or C-terminal of the protein. Non-limiting examples of NLSs include an NLS sequence derived from: the NLS of the SV40 virus large T-antigen, having the amino acid sequence PKKKRKV (SEQ ID NO: 20); the NLS from nucleoplasmin (e.g., the nucleoplasmin bipartite NLS with the sequence KRPAATKKAGQAKKKK (SEQ ID NO: 21)); the c-myc NLS having the amino acid sequence PAAKRVKLD (SEQ ID NO: 22) or RQRRNELKRSP (SEQ ID NO: 23); the hRNPA1 M9 NLS having the sequence NQSSNFGPMKGGNFGGRSSGPYGGGGQYFAKPRNQGGY (SEQ ID NO: 24); the sequence RMRIZFKNKGKDTAELRRRRVEVSVELRKAKKDEQILKRRNV (SEQ ID NO: 25) of the IBB domain from importin-alpha; the sequences VSRKRPRP (SEQ ID NO: 26) and PPKKARED (SEQ ID NO: 27) of the myoma T protein; the sequence PQPKKKPL (SEQ ID NO: 28) of human p53; the sequence SALIKKKKKMAP (SEQ ID NO: 53) of mouse c-abl IV; the sequences DRLRR (SEQ ID NO: 29) and PKQKKRK (SEQ ID NO: 30) of the influenza virus NS1; the sequence RKLKKKIKKL (SEQ ID NO: 31) of the Hepatitis virus delta antigen; the sequence REKKKFLKRR (SEQ ID NO: 32) of the mouse Mx1 protein; the sequence KRKGDEVDGVDEVAKKKSKK (SEQ ID NO: 33) of the human poly(ADP-ribose) polymerase; the sequence RKCLQAGMNLEARKTKK (SEQ ID NO: 34) of the human glucocorticoid receptor; the sequence PKLKRQ (SEQ ID NO: 35) of VACM-1/CUL5; the sequence RPRK (SEQ ID NO: 36) of CXCR4; the sequence RRARRPRG (SEQ ID NO: 37) of VP1; the sequence GKRKLITSEEERSPAKRGRKS (SEQ ID NO: 38) of 53BP1; the sequence KGKKGRTQKEKKAARARSKGKN (SEQ ID NO: 39) of ING4; the sequence RKRCAAGVGGGPAGCPAPGSTPLKKPRR (SEQ ID NO: 40) of IER5; the sequence RKPVTAQERQREREEKRRRRQERAKEREKRRQERER (SEQ ID NO: 41) of ERK5; the sequence RSGGNHRRNGRGGRGGYNRRNNGYHPY (SEQ ID NO: 42) of Hrp1; the sequence TLLLRETMNNLGVSDHAVLSRKTPQPY (SEQ ID NO: 43) of UL79; the sequence of PGKMDKGEHRQERRDRPY (SEQ ID NO: 44) of EWS; the sequence GKKKKGKPGKRREQRKKKRRT (SEQ ID NO: 45) of PTHrP; the sequence SANKVTKNKSNSSPYLNKRKGKPGPDS (SEQ ID NO: 46) of Pho4; the sequence VHSHKKKKIPTSPFFITPKTLTLRRQPKYPRKSAPRRNKLDHY (SEQ ID NO: 47) of rpL23a; the sequence RKHKTNRKPR (SEQ ID NO): 48) MSX1; and the sequence RNKKKK (SEQ ID NO: 54) of NLS-RARα.

In some embodiments, the CRISPR-associated protein comprises at least one (e.g., 1, 2, 3, 4, 5, 6, 7, 8, 9, or 10) Nuclear Export Signal (NES) attached the N-terminal or C-terminal of the protein. In a preferred embodiment a C-terminal and/or N-terminal NLS or NES is attached for optimal expression and nuclear targeting in eukaryotic cells, e.g., human cells.

In some embodiments, the engineered Class 2 type VI Cas13 effectors, such as those substantially lacking collateral activity are mutated at one or more amino acid residues to alter one or more functional activities.

For example, in some embodiments, the engineered Class 2 type VI Cas13 effectors, such as those substantially lacking collateral activity is mutated at one or more amino acid residues to alter its helicase activity.

In some embodiments, the engineered Class 2 type VI Cas13 effectors, such as those substantially lacking collateral activity is mutated at one or more amino acid residues to alter its nuclease activity (e.g., endonuclease activity or exonuclease activity), such as the collateral nuclease activity that is not dependent on guide sequence.

In some embodiments, the engineered Class 2 type VI Cas13 effectors, such as those substantially lacking collateral activity is mutated at one or more amino acid residues to alter its ability to functionally associate with a guide RNA.

In some embodiments, the engineered Class 2 type VI Cas13 effectors, such as those substantially lacking collateral activity is mutated at one or more amino acid residues to alter its ability to functionally associate with a target nucleic acid (such as VEGFA mRNA).

In some embodiments, the engineered Class 2 type VI Cas13 effectors, such as those substantially lacking collateral activity described herein are capable of cleaving a target RNA molecule (such as VEGFA mRNA).

In some embodiments, the engineered Class 2 type VI Cas13 effectors, such as those substantially lacking collateral activity is mutated at one or more amino acid residues to alter its cleaving activity. For example, in some embodiments, the engineered Class 2 type VI Cas13 effectors, such as those substantially lacking collateral activity may comprise one or more mutations that render the enzyme incapable of cleaving a target nucleic acid (such as VEGFA mRNA).

In some embodiments, the engineered Class 2 type VI Cas13 effectors, such as those substantially lacking collateral activity is capable of cleaving the strand of the target nucleic acid that is complementary to the strand to which the guide RNA hybridizes.

In some embodiments, a engineered Class 2 type VI Cas13 effectors, such as those substantially lacking collateral activity described herein can be engineered to have a deletion in one or more amino acid residues to reduce the size of the enzyme while retaining one or more desired functional activities (e.g., nuclease activity and the ability to interact functionally with a guide RNA). The truncated engineered Class 2 type VI Cas13 effectors, such as those substantially lacking collateral activity can be advantageously used in combination with delivery systems having load limitations.

In some embodiments, the engineered Class 2 type VI Cas13 effectors, such as those substantially lacking collateral activity described herein can be fused to one or more peptide tags, including a His-tag, GST-tag, a V5-tag, FLAG-tag, HA-tag, VSV-G-tag, Trx-tag, or myc-tag.

In some embodiments, the engineered Class 2 type VI Cas13 effectors, such as those substantially lacking collateral activity described herein can be fused to a detectable moiety such as GST, a fluorescent protein (e.g., GFP, HcRed, DsRed, CFP, YFP, or BFP), or an enzyme (such as HRP or CAT).

In some embodiments, the engineered Class 2 type VI Cas13 effectors, such as those substantially lacking collateral activity described herein can be fused to MBP, LexA DNA binding domain, or Gal4 DNA-binding domain.

In some embodiments, the engineered Class 2 type VI Cas13 effectors, such as those substantially lacking collateral activity described herein can be linked to or conjugated with a detectable label such as a fluorescent dye, including FITC and DAPI.

In any of the embodiments herein, the linkage between the engineered Class 2 type VI Cas13 effectors, such as those substantially lacking collateral activity described herein and the other moiety can be at the N- or C-terminal of the CRISPR-associated proteins, and sometimes even internally via covalent chemical bonds. The linkage can be effected by any chemical linkage known in the art, such as peptide linkage, linkage through the side chain of amino acids such as D, E, S, T, or amino acid derivatives (Ahx, β-Ala, GABA or Ava), or PEG linkage.

### 3. Polynucleotides and AAV Vector Genomes

One aspect of the invention provides a recombinant adeno-associate virus (rAAV) vector genome, comprising (1) a Cas13X polynucleotide encoding an engineered Cas13X polypeptide of the invention (which substantially lacks guide-RNA independent collateral nuclease activity, but substantially retains guide-RNA-dependent nuclease activity of the original Cas13 protein from which such engineered Cas13X polypeptide derives); and (2) an expression cassette for transcribing a guide RNA targeting a target gene transcript (such as an VEGFA mRNA), wherein the guide RNA includes a functionally linked DR sequence for forming a complex with the Cas13X polypeptide.

More specifically, one aspect of the invention provides a recombinant adeno-associate virus (rAAV) vector genome, comprising: (1) a Cas13X polynucleotide (such as SEQ ID NO: 5) encoding a Cas13X polypeptide, said Cas13X polynucleotide being at least 80%, 85%, 90%, 95%, 96%, 97%, 98%, 99%, 99.2%, 99.4%, 99.6%, 99.8%, or 99.9% identical to SEQ ID NO: 1, said Cas13X polypeptide comprising 1-3 substitutions at Y672, Y676, and/or I751 of SEQ ID NO: 4 (the wt protein encoded by SEQ ID NO: 1), and having substantially the same (e.g., at least about 80%, 90%, 95%, 99% or more) guide RNA-specific nuclease activity as SEQ ID NO: 4 and substantially no (e.g., at most 20%, 15%, 10%, 5%) collateral (guide RNA-independent) nuclease activity of SEQ ID NO: 4; and, (2) a polyA signal sequence 3′ to the Cas13X polynucleotide; optionally, said Cas13X polypeptide has the amino acid sequence of SEQ ID NO: 2 or 3.

Inverted Terminal Repeat (ITR) sequences are important for initiation of viral DNA replication and circularization of adeno-associated virus genomes. Within the ITR sequences, secondary structures (e.g., stems and loops formed by palindromic sequences) are important one or more ITR functions in viral replication and/or packaging. Such sequence elements includes the RBE sequence (Rep binding element), RBE′ sequence, and the trs (terminal resolution sequence).

In certain embodiments, the rAAV vector genome comprises a 5′ AAV ITR sequence and a 3′ AAV ITR sequence.

In certain embodiments, the 5′ and the 3′ AAV ITR sequences are both wild-type AAV ITR sequences from AAV1, AAV2, AAV3A, AAV3B, AAV4, AAV5, AAV6, AAV7, AAVrh74, AAV8, AAV9, AAV10, AAV11, AAV12, AAV13, or a member of the Clade to which any of the AAV1-AAV13 belong.

In certain embodiments, the 5′ and the 3′ AAV ITR sequences are both wild-type AAV ITR sequences from AAV2.

In certain embodiments, the 5′ and/or 3′ ITR sequences are modified ITR sequences. For example, the most 5′ end or the most 3′ end of the wild-type ITR sequences (e.g., AAV2 ITR sequences) may be deleted. The deletion can be up to 18, 17, 16, 15, 14, 13, 12, 11, 10, 9, 8, 7, 6, 5, 4, 3, 2, or 1 nucleotides.

In certain embodiments, up to 15 (such as exactly 15) nucleotides of the most 5′ end nucleotides, and/or up to 15 (such as exactly 15) nucleotides of the most 3′ end nucleotides, of the wild-type AAV2 ITR sequences may be deleted.

Thus the 5′ and/or 3′ modified ITR(s) may comprising up to 144, 143, 142, 141, 140, 139, 138, 137, 136, 135, 134, 133, 132, 131, 130, 129, 128, or 127-nt (such as 130 nucleotides) of the 145-nt wild-type AAV ITR sequences.

In certain embodiments, the modified ITR sequences comprise the RBE sequence, the RBE′ sequence, and/or the trs of the wt ITR sequence.

In certain embodiments, the modified ITR sequences comprise both the RBE sequence and the RBE′ sequence.

In certain embodiments, the modified ITR sequences confer stability of the plasmids of the invention comprising the AAV vector genome (see below) in bacteria, such as stability during plasmid production.

In certain embodiments, the modified ITRs do not interfere with sequencing verification of the plasmids of the invention comprising the AAV vector genome.

In certain embodiments, the modified 5′ ITR sequence comprises a 5′ heterologous sequence that is not part of wild-type AAV 5′ ITR sequence. In certain embodiments, the modified 3′ ITR sequence comprises a 3′ heterologous sequence that is not part of wild-type AAV 3′ ITR sequence.

In certain embodiments, the modified 5′ ITR sequence comprises a 5′ heterologous sequence that is not part of wild-type AAV (e.g., wt AAV2) 5′ ITR sequence, and the modified 3′ ITR sequence comprises a 3′ heterologous sequence that is not part of wild-type AAV (e.g., wt AAV2) 3′ ITR sequence, wherein the 5′ heterologous sequence and the 3′ heterologous sequence are complementary to each other.

In certain embodiments, the 5′ heterologous sequence and the 3′ heterologous sequence each comprises a type II restriction endonuclease recognition sequence, such as recognition sequence for Sse8387I (CCTGCAGG), or recognition sequence for PacI (TTAATTAA).

In certain embodiments, the 5′ heterologous sequence comprises, consists essentially of, or consists of CCTGCAGGCAG (SEQ ID NO: 88), and the 3′ heterologous sequence comprises, consists essentially of, or consists of the reverse complement of SEQ ID NO: 88. An exemplary 5′ ITR comprising SEQ ID NO: 88 is SEQ ID NO: 10. An exemplary 3′ ITR comprising the reverse complement of SEQ ID NO: 88 is SEQ ID NO: 11.

In certain embodiments, the 5′ heterologous sequence comprises, consists essentially of, or consists of TTAATTAAGG (SEQ ID NO: 89), and the 3′ heterologous sequence comprises, consists essentially of, or consists of the reverse complement of SEQ ID NO: 89.

In certain embodiments, the 5′ ITR and the 3′ ITR are both flip ITR's.

In certain embodiments, the 5′ ITR and the 3′ ITR are both flop ITR's.

In certain embodiments, the 5′ ITR and the 3′ ITR are independently flip or flop ITR's.

In certain embodiments, the 5′ ITR is a flip ITR, and the 3′ ITR is a flop ITR.

In certain embodiments, the 5′ ITR is a flop ITR, and the 3′ ITR is a flip ITR.

In certain embodiments, the 5′ ITR is a flip ITR, and the 3′ ITR is a flip ITR.

In certain embodiments, the 5′ ITR is a flop ITR, and the 3′ ITR is a flop ITR.

As used herein, a 5′ flip ITR has the B:B′ segment closer to the 5′-terminal than the C:C′ segment. A 3′ flip ITR has the B:B′ segment closer to the 3′-terminal than the C:C′ segment. A 5′ flop ITR has the C:C′ segment closer to the 5′-terminal than the B:B′ segment. A 3′ flop ITR has the C:C′ segment closer to the 3′-terminal than the B:B′ segment.

In certain embodiments, the modified 5′ ITR and the modified 3′ ITR are both flop ITRs, the modified 5′ ITR comprises a 5′ heterologous sequence that is not part of wild-type AAV2 5′ ITR sequence (such as SEQ ID NO: 88 or 89), and the modified 3′ ITR sequence comprises a 3′ heterologous sequence that is not part of wild-type AAV2 3′ ITR sequence, wherein the 5′ heterologous sequence and the 3′ heterologous sequence are complementary to each other, and each comprises a type II restriction endonuclease recognition sequence, such as recognition sequence for Sse83871 or PacI; optionally, said modified 5′ ITR sequence further comprises a deletion in the C:C′ segment, such as an 11-nts deletion AAAGCCCGGGC (SEQ ID NO: 90).

In certain embodiments, the 5′ ITR comprises up to 141 nts of the most 3′ nucleotides of the 145-nt wt AAV2 5′ ITR (e.g., a deletion of 4 or more most 5′ end of the 145-nt wt AAV2 5′ ITR).

In certain embodiments, the 5′ ITR comprises up to 130 nts of the most 3′ nucleotides of the 145-nt wt AAV2 5′ ITR (e.g., a deletion of 15 or more most 5′ end of the 145-nt wt AAV2 5′ ITR).

In certain embodiments, the 3′ ITR comprises up to 141 nts of the most 5′ nucleotides of the 145-nt wt AAV2 3′ ITR (e.g., a deletion of 4 or more most 3′ end of the 145-nt wt AAV2 3′ ITR).

In certain embodiments, the 3′ ITR comprises up to 130 nts of the most 5′ nucleotides of the 145-nt wt AAV2 3′ ITR (e.g., a deletion of 15 or more most 3′ end of the 145-nt wt AAV2 3′ ITR).

In certain embodiments, the 5′ and 3′ ITR sequences are compatible for AAV production in mammalian-cell based on triple transfection.

In certain embodiments, the 5′ and 3′ ITR sequences are compatible for AAV production in insect cell (e.g., Sf9) based on baculovirus vector (see below).

In certain embodiments, the 5′ and 3′ ITR sequences are compatible for AAV production in mammalian-cell based on HSV vectors (see below).

In some embodiments, the Cas13X polynucleotide is operably linked to a regulatory element (e.g., a promoter) in order to control the expression of the Cas13X polypeptide. In some embodiments, the promoter is ubiquitous. In some embodiments, the promoter is a constitutive promoter. In some embodiments, the promoter is an inducible promoter. In some embodiments, the promoter is a cell-specific promoter. In some embodiments, the promoter is an organism-specific promoter, e.g., tissue-specific promoter.

Suitable promoters are known in the art and include, for example, a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter. For example, a U6 promoter can be used to regulate the expression of a guide RNA molecule described herein. In some embodiments, the elongation factor 1α short (EFS) promoter can be used to regulate the expression of the Cas13 effector protein described herein.

In certain embodiments, the promoter is the elongation factor 1α short (EFS) promoter, such as SEQ ID NO: 12.

In certain embodiments, the rAAV vector genome of the invention further comprises a coding sequence for a nuclear localization sequence (NLS) fused N-terminal, C-terminal, and/or internally to the Cas13X polypeptide, and/or a coding sequence for a nuclear export signal (NES) fused N-terminal, C-terminal, and/or internally to the Cas13X polypeptide.

In certain embodiments, the rAAV vector genome of the invention comprises a first NLS coding sequence 5′ to the Cas13X polynucleotide, and/or a second NLS coding sequence 3′ to the Cas13X polynucleotide (e.g., comprising both the first and the second NLS coding sequences).

In certain embodiments, the NLS, the first NLS, and the second NLS is independently selected from the group consisting of SEQ ID NOs: 20-48 or 53-54.

In certain embodiments, the rAAV vector genome of the invention further comprises a Kozak sequence or a functional variant thereof. In certain embodiments, the Kozak sequence is SEQ ID NO: 13; or a sequence comprising at most 1, 2, 3, or 4 nucleotide differences from SEQ ID NO: 13 other than the ATG start codon within the Kozak sequence, wherein the last three nucleotide is optionally ACC or GCC.

In certain embodiments, the rAAV vector genome of the invention further comprises a polyadenylation (polyA) signal sequence. In certain embodiments, the polyA signal sequence is selected from the group consisting of growth hormone polyadenylation signal (bGH polyA), a small polyA signal (SPA), a human growth hormone polyadenylation signal (hGH polyA), a SV40 polyA signal (SV40 polyA), a rabbit beta globin polyA signal (rBG polyA), or a variant thereof. In certain embodiments, the polyA signal sequence is SV40 polyA signal sequence or a functional variant thereof (such as SEQ ID NO: 15).

In certain embodiments, the expression cassette for transcribing a guide RNA targeting the target gene transcript (e.g., VEGFA mRNA) comprises an RNA pol III promoter, wherein said second transcription unit is 3′ to the Cas13X polynucleotide.

In certain embodiments, the RNA pol III promoter is U6 (such as SEQ ID NO: 16), H1, 7SK, or a variant thereof.

In certain embodiments, the expression cassette for transcribing the guide RNA targeting the target gene transcript encodes one or more (e.g., 2 or 3) single guide RNAs (sgRNAs) each complementary to a target RNA sequence (e.g., VEGFA mRNA), and each capable of directing said Cas13X polypeptide to cleave said target RNA; optionally, each said sgRNA comprises a direct repeat (DR) sequence that binds the Cas13X polypeptide. More detailed description for the DR sequence and sgRNA/crRNA are provided in a separate section below (incorporated herein by reference).

In certain embodiments, the one or more sgRNAs comprise SEQ ID NOs: 7 and 8.

In certain embodiments, the DR sequence is a nucleic sequence having at least 90% identity to SEQ ID NO: 6, at most 1, 2, 3, 4, or 5 nucleotide differences from SEQ ID NO: 6, and/or substantially the same secondary structure as that of SEQ ID NO: 6.

In certain embodiments, the DR sequence comprises, consists essentially of, or consists of SEQ ID NO: 6.

In certain embodiments, the target RNA is a transcript (e.g., mRNA) of a target gene associated with an eye disease or disorder.

In certain embodiments, the eye disease or disorder is amoebic keratitis, fungal keratitis, bacterial keratitis, viral keratitis, onchorcercal keratitis, keratoconjunctivitis, bacterial keratoconjunctivitis, viral keratoconjunctivitis, vernal keratoconjunctivitis, atopic keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, noninfectious uveitis, anterior uveitis, posterior uveitis (including toxoplasmosis), pan-uveitis, an inflammatory disease of the vitreous or retina, endophthalmitis prophylaxis and treatment, macular edema, macular degeneration, wet age related macular degeneration (wet AMD), dry age related macular degeneration (dry AMD), diabetic macular edema (DME), allergic conjunctivitis, proliferative and non-proliferative diabetic retinopathy, hypertensive retinopathy, an autoimmune disease of the retina, primary and metastatic intraocular melanoma, other intraocular metastatic tumors, open angle glaucoma, Stargardt's disease, Fundus Flavimaculatus, closed angle glaucoma, pigmentary glaucoma, retinitis pigmentosa (RP), Leber's congenital amaurosis (LCA), Usher's syndrome, choroideremia, a rod-cone or cone-rod dystrophy, a ciliopathy, a mitochondrial disorder, progressive retinal atrophy, a degenerative retinal disease, geographic atrophy, a familial or acquired maculopathy, a retinal photoreceptor disease, a retinal pigment epithelial-based disease, cystoid macular edema, retinal detachment, traumatic retinal injury, iatrogenic retinal injury, macular holes, macular telangiectasia, a ganglion cell disease, an optic nerve cell disease, optic neuropathy, ischemic retinal disease, retinopathy of prematurity, retinal vascular occlusion, familial macroaneurysm, a retinal vascular disease, an ocular vascular diseases, a vascular disease, an ischemic optic neuropathy disease, diabetic retinal oedema, senile macular degeneration due to sub-retinal neovascularization, myopic retinopathy, retinal ischemia, choroidal vascular insufficiency, choroidal thrombosis and neovascular retinopathies resulting from carotoid artery ischemia, corneal neovascularisation, a corneal disease or opacification with an exudative or inflammatory component, diffuse lamellar keratitis, neovascularisation due to penetration of the eye or contusive ocular injury, rubosis iritis, Fuchs' heterochromic iridocyclitis, chronic uveitis, anterior uveitis, inflammatory conditions resulting from surgeries such as LASIK, LASEK, refractive surgery, IOL implantation; irreversible corneal oedema as a complication of cataract surgery, oedema as a result of insult or trauma, inflammation, infectious and non-infectious conjunctivitis, iridocyclitis, iritis, scleritis, episcleritis, superficial punctuate keratitis, keratoconus, posterior polymorphous dystrophy, Fuch's dystrophies, aphakic and pseudophakic bullous keratopathy, corneal oedema, scleral disease, ocular cicatrcial pemphigoid, pars planitis, Posner Schlossman syndrome, Behcet's disease, Vogt-Koyanagi-Harada syndrome, hypersensitivity reactions, ocular surface disorders, conjunctival oedema, Toxoplasmosis chorioretinitis, inflammatory pseudotumor of the orbit, chemosis, conjunctival venous congestion, periorbiatal cellulits, acute dacroycystitis, non-specific vasculitis, sarcoidosis, cytomegalovirus infection, and combinations thereof.

In certain embodiments, the eye disease or disorder is wet age related macular degeneration (wet AMD).

In certain embodiments, the target gene is selected from the group consisting of Vascular Endothelial Growth Factor A (VEGFA), complement factor H (CFH), age-related maculopathy susceptibility 2 (ARMS2), HtrA serine peptidase 1 (HTRA1), ATP Binding Cassette Subfamily A Member 4 (ABCA4), Peripherin-2 (PRPH2), fibulin-5 (FBLN5), ERCC Excision Repair 6 Chromatin Remodeling Factor (ERCC6), Retina And Anterior Neural Fold Homeobox 2 (RAX2), Complement C3 (C3), Toll Like Receptor 4 (TLR4), Cystatin C (CST3), CX3C Chemokine Receptor 1 (CX3CR1), complement factor I (CFI), Complement C2 (C2), Complement Factor B (CFB), Complement C9 (C9), Mitochondrially Encoded TRNA Leucine 1 (UUA/G) (MT-TL-1), Complement Factor H Related 1 (CFHR1), Complement Factor H Related 3 (CFHR3), Ciliary Neurotrophic Factor (CNTF), pigment epithelium-derived factor (PEDF), rod-derived cone viability factor (RdCVF), glial-derived neurotrophic factor (GDNF), Myosin VIIA (MYO7A); Centrosomal Protein 290 (CEP290), Cadherin Related 23 (CDH23), Eyes Shut Homolog (EYS), Usherin (USH2A), adhesion G protein-coupled receptor V1 (ADGRV1), ALMS1 Centrosome And Basal Body Associated Protein (ALMS1), Retinoid Isomerohydrolase 65 kDa (RPE65), Aryl-hydrocarbon-interacting protein-like 1 (AIPL1), Guanylate Cyclase 2D, Retinal (GUCY2D), Leber Congenital Amaurosis 5 Protein (LCA5), Cone-Rod Homeobox (CRX), Clarin (CLRN1), ATP Binding Cassette Subfamily A Member 4 (ABCA4), Retinol Dehydrogenase 12 (RDH12), Inosine Monophosphate Dehydrogenase 1 (IMPDH1), Crumbs Cell Polarity Complex Component 1 (CRB1), Lecithin retinol acyltransferase (LRAT), Nicotinamide Nucleotide Adenylyltransferase 1 (NMNAT1), TUB Like Protein 1 (TULP1), MER Proto-Oncogene, Tyrosine Kinase (MERTK), Retinitis Pigmentosa GTPase Regulato (RPGR), RP2 Activator Of ARL3 GTPase (RP2), X-linked retinitis pigmentosa GTPase regulator-interacting protein 1 (RPGRIP), Cyclic Nucleotide Gated Channel Subunit Alpha 3 (CNGA3), Cyclic Nucleotide Gated Channel Subunit Beta 3 (CNGB3), G Protein Subunit Alpha Transducin 2 (GNAT2), Fibroblast Growth Factor 2 (FGF2), Erythropoietin (EPO), BCL2 Apoptosis Regulator (BCL2), BCL2 Like 1 (BCL2L1), Nuclear Factor Kappa B (NFκB), Endostatin, Angiostatin, fms-like tyrosine kinase receptor (sFlt), Pigment-dispersing factor receptor (Pdfr), Interleukin 10 (IL10), soluble interleukin 17 (sIL17R), Interleukin-1-receptor antagonist (IL1-ra), TNF Receptor Superfamily Member 1A (TNFRSF1A), TNF Receptor Superfamily Member 1B (TNFRSF1B), and interleukin 4 (IL4).

In certain embodiments, the target gene is VEGFA.

In certain embodiments, the target RNA is a transcript (e.g., mRNA) of a target gene associated with a neurodegenerative disease or disorder.

In certain embodiments, the neurodegenerative disease or disorder is alcoholism, Alexander's disease, Alper's disease, Alzheimer's Disease, amyotrophic lateral sclerosis (ALS), ataxia telangiectasia, neuronal ceroid lipofuscinoses, Batten disease, bovine spongiform encephalopathy (BSE), Canavan disease, cerebral palsy, Cockayne syndrome, corticobasal degeneration, Creutzfeldt-Jakob disease, frontotemporal lobar degeneration, Huntington's disease, HIV-associated dementia, Kennedy's disease, Lewy body dementia, neuroborreliosis, primary age-related tauopathy (PART)/Neurofibrillary tangle-predominant senile dementia, Machado-Joseph disease, multiple system atrophy, multiple sclerosis, multiple sulfatase deficiency, mucolipidoses, narcolepsy, Niemann Pick disease, Parkinson's Disease, Pick's disease, Pompe disease, primary lateral sclerosis, prion diseases, neuronal loss, cognitive defect, motor neuron diseases, Duchenne Muscular Dystrophy (DMD), frontotemporal dementia, frontotemporal dementia and parkinsonism linked to chromosome 17, Lytico-Bodig disease (Parkinson-dementia complex of Guam), neuroaxonal dystrophies, Refsum's disease, Schilder's disease, subacute combined degeneration of spinal cord secondary to pernicious anaemia, Spielmeyer-Vogt-Sjogren-Batten disease, Parkinsonism linked to chromosome 17 (FTDP-17), Prader Willi syndrome, Myotonic dystrophy, chronic traumatic encephalopathy including dementia pugilistica, spinocerebellar ataxia, spinal muscular atrophy, Steele-Richardson-Olszewski disease, Tabes dorsalis, Niemann-Pick Type C (NPC1 and/or NPC2 defect), Smith-Lemli-Opitz Syndrome (SLOS), an inborn error of cholesterol synthesis, Tangier disease, Pelizaeus-Merzbacher disease, a neuronal ceroid lipofuscinosis, a primary glycosphingolipidosis, Farber disease or multiple sulphatase deficiency, Gaucher disease, Fabry disease, GM1 gangliosidosis, GM2 gangliosidosis, Krabbe disease, metachromatic leukodystrophy (MLD), NPC, GM1 gangliosidosis, Fabry disease, a neurodegenerative mucopolysaccharidosis, MPS I, MPS IH, MPS IS, MPS II, MPS III, MPS IIIA, MPS IIIB, MPS IIIC, MPS HID, MPS, IV, MPS IV A, MPS IV B, MPS VI, MPS VII, MPS IX, a disease with secondary lysosomal involvement, SLOS, Tangier disease, ganglioglioma, gangliocytoma, meningioangiomatosis, postencephalitic parkinsonism, subacute sclerosing panencephalitis, lead encephalopathy, tuberous sclerosis, Hallervorden-Spatz disease, lipofuscinosis, cerebellar ataxia, parkinsonism, Louis-Barr syndrome, multiple systems atrophy, fronto-temporal dementia or lower body Parkinson's syndrome, Niemann Pick disease, Niemann Pick type C, Niemann Pick type A, Tay-Sachs disease, multisystemic atrophy cerebellar type (MSA-C), fronto-temporal dementia with parkinsonism, progressive supranuclear palsy, cerebellar downbeat nystagmus, Sandhoff's disease or mucolipidosis type II, or combinations thereof.

In certain embodiments, the target RNA is a transcript (e.g., mRNA) of a target gene associated with a cancer.

In certain embodiments, the cancer is carcinomas, sarcomas, myelomas, leukemias, lymphomas and mixed type tumors. Non-limiting examples of cancers that may treated by methods and compositions described herein include, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; plasmacytoma, colorectal cancer, rectal cancer, and hairy cell leukemia.

In certain embodiments, the rAAV vector genome of the invention comprises an ITR-to-ITR polynucleotide (such as SEQ ID NO: 17) comprising, from 5′ to 3′: (a) a 5′ ITR from AAV2 (such as SEQ ID NO: 10); (b) an EFS promoter (such as SEQ ID NO: 12); (c) a KOZAK sequence (such as SEQ ID NO: 13); (d) a first SV40 NLS coding sequence (such as SEQ ID NO: 14); (e) the Cas13X polynucleotide (such as SEQ ID NO: 5) encoding the Cas13X polypeptide of SEQ ID NO: 2 or 3; (f) a second SV40 NLS coding sequence (such as SEQ ID NO: 14); (g) an SV40 polyA signal sequence (such as SEQ ID NO: 15); (h) a U6 promoter (such as SEQ ID NO: 16); (i) a first direct repeat sequence (such as SEQ ID NO: 6); (j) an sg1 coding sequence specific for VEGFA (such as SEQ ID NO: 7); (k) a second direct repeat sequence (such as SEQ ID NO: 6); (l) an sg2 coding sequence specific for VEGFA (such as SEQ ID NO: 8); (m) a third direct repeat sequence (such as SEQ ID NO: 6); and, (n) a 3′ ITR from AAV2 (such as SEQ ID NO: 11); or a polynucleotide at least about 90%, 91%, 92%, 93%, 94%, 95%, 96%, 97%, 98%, 99%, 99.5%, 99.6%, 99.7%, 99.8%, 99.9% identical to said ITR-to-ITR polynucleotide.

In certain embodiments, the recombinant AAV (rAAV) vector genome comprises, consists essentially of, or consists of SEQ ID NO: 17, or a polynucleotide at least 90%, 91%, 92%, 93%, 94%, 95%, 96%, 97%, 98%, 99%, 99.5%, 99.6%, 99.7%, 99.8%, 99.9% identical thereto, wherein said polynucleotide encodes a Cas13X polypeptide at least 80%, 85%, 90%, 95%, 96%, 97%, 98%, or 99% identical to SEQ ID NO: 4, and an sgRNA specific for VEGFA, wherein said Cas13X polypeptide comprises 1-3 substitutions at Y672, Y676, and/or I751 of SEQ ID NO: 4, and wherein said sgRNA forms a complex with said Cas13X polypeptide and directs said Cas13X polypeptide to cleave a VEGFA mRNA transcript with substantially the same (e.g., at least about 80%, 90%, 95%, 99% or more) guide RNA-specific nuclease activity as SEQ ID NO: 4 and substantially no (e.g., at most 20%, 15%, 10%, 5%) collateral (guide RNA-independent) nuclease activity of SEQ ID NO: 4.

In certain embodiments, the rAAV vector genome is SEQ ID NO: 17, or the polynucleotide at least 95% or 99% identical thereto. In certain embodiments, the rAAV vector genome is SEQ ID NO: 17.

In some embodiments, the rAAV vector genome is present in a vector (e.g., a viral vector or a phage, such as an HSV vector, a baculovirus vector, or an AAV vector). The vector can be a cloning vector, or an expression vector. The vectors can be plasmids, phagemids, Cosmids, etc. The vectors may include one or more regulatory elements that allow for the propagation of the vector in a cell of interest (e.g., a bacterial cell, insect cell, or a mammalian cell). In some embodiments, the vector includes a nucleic acid encoding a single component of a CRISPR-associated (Cas) system described herein. In some embodiments, the vector includes multiple nucleic acids, each encoding a component of a CRISPR-associated (Cas) system described herein.

In one aspect, the present disclosure provides nucleic acid sequences that are at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequences described herein, e.g., nucleic acid sequences (such as ITR-to-ITR sequences, for example, SEQ ID NO: 17) encoding the engineered Class 2 type VI Cas13 protein substantially lacking collateral activity, derivatives, functional fragments, and comprising transcription cassette for guide/crRNA, including the DR sequences.

In certain embodiments, the Cas13X polynucleotide sequence of the invention encodes amino acid sequences that are at least 50%, 55%, 60%, 65%, 70%, 75%, 80%, 85%, 90%, 91%, 92%, 93%, 94%, 95%, 96%, 97%, 98%, 99%, or 100% identical to the amino acid sequences of the subject engineered Class 2 type VI Cas13 protein substantially lacking collateral activity (e.g., SEQ ID NO: 2 or 3).

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as the sequences described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In related embodiments, the invention provides amino acid sequences having at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

The proteins described herein (e.g., an engineered Class 2 type VI Cas13 protein substantially lacking collateral activity) can be delivered or used as either nucleic acid molecules or polypeptides.

In certain embodiments, the nucleic acid molecule encoding the engineered Class 2 type VI Cas13 protein, such as those substantially lacking collateral activity, derivatives or functional fragments thereof are codon-optimized for expression in a host cell or organism. The host cell may include established cell lines (such as HeLa, 293, or 293T cells) or isolated primary cells. The nucleic acid can be codon optimized for use in any organism of interest, in particular human cells or bacteria. For example, the nucleic acid can be codon-optimized for any prokaryotes (such as E. coli), or any eukaryotes such as human and other non-human eukaryotes including yeast, worm, insect, plants and algae (including food crop, rice, corn, vegetables, fruits, trees, grasses), vertebrate, fish, non-human mammal (e.g., mice, rats, rabbits, dogs, birds (such as chicken), livestock (cow or cattle, pig, horse, sheep, goat etc.), or non-human primates). Codon usage tables are readily available, for example, at the “Codon Usage Database” available at www.kazusa.orjp/codon/, and these tables can be adapted in a number of ways. See Nakamura et al., Nucl. Acids Res. 28:292, 2000 (incorporated herein by reference in its entirety). Computer algorithms for codon optimizing a particular sequence for expression in a particular host cell are also available, such as Gene Forge (Aptagen; Jacobus, Pa.).

An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at http://www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a Cas correspond to the most frequently used codon for a particular amino acid.

4. RNA Guides or crRNA

In some embodiments, the CRISPR systems described herein include at least RNA guide (e.g., a gRNA or a crRNA). Such guide RNA may be encoded by the same AAV vector genome encoding the engineered Cas13X polypeptide (see FIG. 2).

The architecture of multiple RNA guides is known in the art (see, e.g., International Publication NOs. WO 2014/093622 and WO 2015/070083, the entire contents of each of which are incorporated herein by reference).

In some embodiments, the CRISPR systems described herein include multiple RNA guides (e.g., one, two, three, four, five, six, seven, eight, or more RNA guides).

In some embodiments, the RNA guide includes a crRNA. In some embodiments, the RNA guide includes a crRNA but not a tracrRNA.

Sequences for guide RNAs from multiple CRISPR systems are generally known in the art, see, for example, Grissa et al. (Nucleic Acids Res. 35 (web server issue): W52-7, 2007; Grissa et al., BMC Bioinformatics 8:172, 2007; Grissa et al., Nucleic Acids Res. 36 (web server issue): W145-8, 2008; and Moller and Liang, PeerJ 5: e3788, 2017; the CRISPR database at: crispr.i2bc.paris-saclayfr/crispr/BLAST/CRISPRsBlast.php; and MetaCRAST available at: github.com/molleraj/MetaCRAST). All incorporated herein by reference.

In some embodiments, the crRNA includes a direct repeat (DR) sequence and a spacer sequence. In certain embodiments, the crRNA comprises, consists essentially of, or consists of a direct repeat sequence linked to a guide sequence or spacer sequence, preferably at the 3′-end of the spacer sequence.

In general, an engineered Class 2 type VI Cas13 protein, such as those substantially lacking collateral activity forms a complex with the mature crRNA, which spacer sequence directs the complex to a sequence-specific binding with the target RNA that is complementary to the spacer sequence, and/or hybridizes to the spacer sequence. The resulting complex comprises the engineered Class 2 type VI Cas13 protein, such as those substantially lacking collateral activity and the mature crRNA bound to the target RNA.

The direct repeat sequences for the Cas13 systems are generally well conserved, especially at the ends, with, for example, a GCTG for Cas13e and GCTGT for Cas13f at the 5′-end, reverse complementary to a CAGC for Cas13e and ACAGC for Cas13f at the 3′ end. This conservation suggests strong base pairing for an RNA stem-loop structure that potentially interacts with the protein(s) in the locus.

In some embodiments, the direct repeat sequence, when in RNA, comprises the general secondary structure of 5′-Sla-Ba-S2a-L-S2b-Bb-S1b-3′, wherein segments S1a and S1b are reverse complement sequences and form a first stem (S1) having 4 nucleotides in Cas13e and 5 nucleotides in Cas13f; segments Ba and Bb do not base pair with each other and form a symmetrical or nearly symmetrical bulge (B), and have 5 nucleotides each in Cas13e, and 5 (Ba) and 4 (Bb) or 6 (Ba) and 5 (Bb) nucleotides respectively in Cas13f; segments S2a and S2b are reverse complement sequences and form a second stem (S2) having 5 base pairs in Cas13e and either 6 or 5 base pairs in Cas13f; and L is an 8-nucleotide loop in Cas13e and a 5-nucleotide loop in Cas13f.

In certain embodiments, Sla has a sequence of GCUG in Cas13e and GCUGU in Cas13f.

In certain embodiments, S2a has a sequence of GCCCC in Cas13e and A/G CCUC G/A in Cas13f (wherein the first A or G may be absent).

In some embodiments, the direct repeat sequence comprises or consists of a nucleic acid sequence of SEQ ID NO: 6.

As used herein, “direct repeat sequence” may refer to the DNA coding sequence in the CRISPR locus, or to the RNA encoded by the same in crRNA. Thus when any of SEQ ID NO: 6 is referred to in the context of an RNA molecule, such as crRNA, each T is understood to represent a U.

In some embodiments, the direct repeat sequence comprises or consists of a nucleic acid sequence having up to 1, 2, 3, 4, 5, 6, 7, or 8 nucleotides of deletion, insertion, or substitution of SEQ ID NO: 6. In some embodiments, the direct repeat sequence comprises or consists of a nucleic acid sequence having at least 80%, 85%, 90%, 95%, or 97% of sequence identity with SEQ ID NO: 6 (e.g., due to deletion, insertion, or substitution of nucleotides in SEQ ID NO: 6). In some embodiments, the direct repeat sequence comprises or consists of a nucleic acid sequence that is not identical to any one of SEQ ID NO: 6, but can hybridize with a complement of any one of SEQ ID NO: 6 under stringent hybridization conditions, or can bind to a complement of any one of SEQ ID NO: 6 under physiological conditions.

In certain embodiments, the deletion, insertion, or substitution does not change the overall secondary structure of that of SEQ ID NO: 6 (e.g., the relative locations and/or sizes of the stems and bulges and loop do not significantly deviate from that of the original stems, bulges, and loop). For example, the deletion, insert, or substitution may be in the bulge or loop region so that the overall symmetry of the bulge remains largely the same. The deletion, insertion, or substitution may be in the stems so that the length of the stems do not significantly deviate from that of the original stems (e.g., adding or deleting one base pair in each of the two stems correspond to 4 total base changes).

In certain embodiments, the deletion, insertion, or substitution results in a derivative DR sequence that may have t 1 or 2 base pair(s) in one or both stems, have t 1, 2, or 3 bases in either or both of the single strands in the bulge, and/or have t 1, 2, 3, or 4 bases in the loop region.

In certain embodiments, any of the above direct repeat sequences that is different from any one of SEQ ID NO: 6 retains the ability to function as a direct repeat sequence in the Cas13e proteins, as the DR sequence of SEQ ID NO: 6.

In some embodiments, the direct repeat sequence comprises or consists of a nucleic acid having a nucleic acid sequence of any one of SEQ ID NO: 6, with a truncation of the initial three, four, five, six, seven, or eight 3′ nucleotides.

In classic CRISPR systems, the degree of complementarity between a guide sequence (e.g., a crRNA) and its corresponding target sequence can be about 50%, 60%, 75%, 80%, 85%, 90%, 95%, 97.5%, 99%, or 100%. In some embodiments, the degree of complementarity is 90-100%.

The guide RNAs can be about 5, 10, 11, 12, 13, 14, 15, 16, 17, 18, 19, 20, 21, 22, 23, 24, 25, 26, 27, 28, 29, 30, 35, 40, 45, 50, 75, 100, 125, 150, 175, 200 or more nucleotides in length. For example, for use in a functional engineered Cas13e effector protein, or homologs, orthologs, derivatives, fusions, conjugates, or functional fragment thereof, the spacer can be between 10-60 nucleotides, 20-50 nucleotides, 25-45 nucleotides, 25-35 nucleotides, or about 27, 28, 29, 30, 31, 32, or 33 nucleotides. For use in dCas version of any of the above, however, the spacer can be between 10-200 nucleotides, 20-150 nucleotides, 25-100 nucleotides, 25-85 nucleotides, 35-75 nucleotides, 45-60 nucleotides, or about 46, 47, 48, 49, 50, 51, 52, 53, 54, or 55 nucleotides.

To reduce off-target interactions, e.g., to reduce the guide interacting with a target sequence having low complementarity, mutations can be introduced to the CRISPR systems so that the CRISPR systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

It is known in the field that complete complementarity is not required, provided there is sufficient complementarity to be functional. Modulations of cleavage efficiency can be exploited by introduction of mismatches, e.g., one or more mismatches, such as 1 or 2 mismatches between spacer sequence and target sequence, including the position of the mismatch along the spacer/target. The more central (i.e., not at the 3′ or 5′-ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

Type VI CRISPR-Cas effectors have been demonstrated to employ more than one RNA guide, thus enabling the ability of these effectors, and systems and complexes that include them, to target multiple nucleic acids. In some embodiments, the CRISPR systems comprising the engineered Class 2 type VI Cas13 protein, such as those substantially lacking collateral activity, as described herein, include multiple RNA guides (e.g., two, three, four, five, six, seven, eight, nine, ten, fifteen, twenty, thirty, forty, or more) RNA guides. In some embodiments, the CRISPR systems described herein include a single RNA strand or a nucleic acid encoding a single RNA strand, wherein the RNA guides are arranged in tandem. The single RNA strand can include multiple copies of the same RNA guide, multiple copies of distinct RNA guides, or combinations thereof. The processing capability of the Type VI-E and VI-F CRISPR-Cas effector proteins described herein enables these effectors to be able to target multiple target nucleic acids (e.g., target RNAs) without a loss of activity. In some embodiments, the Type VI-E and VI-F CRISPR-Cas effector proteins may be delivered in complex with multiple RNA guides directed to different target RNA. In some embodiments, the engineered Class 2 type VI Cas13 protein, such as those substantially lacking collateral activity may be co-delivered with multiple RNA guides, each specific for a different target nucleic acid. Methods of multiplexing using CRISPR-associated proteins are described, for example, in U.S. Pat. No. 9,790,490 B2, and EP 3009511 B1, the entire contents of each of which are expressly incorporated herein by reference.

The spacer length of crRNAs can range from about 10-50 nucleotides, such as 15-50 nucleotides, 20-50 nucleotides, 25-50 nucleotide, or 19-50 nucleotides. In some embodiments, the spacer length of a guide RNA is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides (e.g., 15, 16, or 17 nucleotides), from 17 to 20 nucleotides (e.g., 17, 18, 19, or 20 nucleotides), from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 36, 37, 38, 39, 40, 41, 42, 43, 44, or 45 nucleotides), from 30 or 35 to 40 nucleotides, from 41 to 45 nucleotides, from 45 to 50 nucleotides (e.g., 45, 46, 47, 48, 49, or 50 nucleotides), or longer. In some embodiments, the spacer length is from about 15 to about 42 nucleotides.

In some embodiments, the direct repeat length of the guide RNA is 15-36 nucleotides, is at least 16 nucleotides, is from 16 to 20 nucleotides (e.g., 16, 17, 18, 19, or 20 nucleotides), is from 20-30 nucleotides (e.g., 20, 21, 22, 23, 24, 25, 26, 27, 28, 29, or 30 nucleotides), is from 30-40 nucleotides (e.g., 30, 31, 32, 33, 34, 35, 36, 37, 38, 39, or 40 nucleotides), or is about 36 nucleotides (e.g., 33, 34, 35, 36, 37, 38, or 39 nucleotides). In some embodiments, the direct repeat length of the guide RNA is 36 nucleotides.

In some embodiments, the overall length of the crRNA/guide RNA is about 36 nucleotides longer than any one of the spacer sequence length described herein above. For example, the overall length of the crRNA/guide RNA may be between 45-86 nucleotides, or 60-86 nucleotides, 62-86 nucleotides, or 63-86 nucleotides.

The crRNA sequences can be modified in a manner that allows for formation of a complex between the crRNA and the engineered Class 2 type VI Cas13 protein, such as those substantially lacking collateral activity, and successful binding to the target, while at the same time not allowing for successful nuclease activity (i.e., without nuclease activity/without causing indels). These modified guide sequences are referred to as “dead crRNAs,” “dead guides,” or “dead guide sequences.” These dead guides or dead guide sequences may be catalytically inactive or conformationally inactive with regard to nuclease activity. Dead guide sequences are typically shorter than respective guide sequences that result in active RNA cleavage. In some embodiments, dead guides are 5%, 10%, 20%, 30%, 40%, or 50%, shorter than respective guide RNAs that have nuclease activity. Dead guide sequences of guide RNAs can be from 13 to 15 nucleotides in length (e.g., 13, 14, or 15 nucleotides in length), from 15 to 19 nucleotides in length, or from 17 to 18 nucleotides in length (e.g., 17 nucleotides in length).

In some embodiments, the guide RNA comprises SEQ ID NO: 7 and/or 8.

Thus, in one aspect, the disclosure provides non-naturally occurring or engineered CRISPR systems including a functional engineered Class 2 type VI Cas13 protein, such as those substantially lacking collateral activity as described herein, and a crRNA, wherein the crRNA comprises a dead crRNA sequence whereby the crRNA is capable of hybridizing to a target sequence such that the CRISPR system is directed to a target RNA of interest in a cell without detectable nuclease activity (e.g., RNase activity).

A detailed description of dead guides is described, e.g., in International Publication No. WO 2016/094872, which is incorporated herein by reference in its entirety.

Guide RNAs (e.g., crRNAs) can be generated as components of inducible systems. The inducible nature of the systems allows for spatio-temporal control of gene editing or gene expression. In some embodiments, the stimuli for the inducible systems include, e.g., electromagnetic radiation, sound energy, chemical energy, and/or thermal energy.

In some embodiments, the transcription of guide RNA (e.g., crRNA) can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in the entirety.

The sequences and the lengths of the RNA guides (e.g., crRNAs) described herein can be optimized. In some embodiments, the optimized length of an RNA guide can be determined by identifying the processed form of crRNA (i.e., a mature crRNA), or by empirical length studies for crRNA tetraloops.

The crRNAs can also include one or more aptamer sequences. Aptamers are oligonucleotide or peptide molecules have a specific three-dimensional structure and can bind to a specific target molecule. The aptamers can be specific to gene effectors, gene activators, or gene repressors. In some embodiments, the aptamers can be specific to a protein, which in turn is specific to and recruits and/or binds to specific gene effectors, gene activators, or gene repressors. The effectors, activators, or repressors can be present in the form of fusion proteins. In some embodiments, the guide RNA has two or more aptamer sequences that are specific to the same adaptor proteins. In some embodiments, the two or more aptamer sequences are specific to different adaptor proteins. The adaptor proteins can include, e.g., MS2, PP7, Qβ, F2, GA, fr, JP501, M12, R17, BZ13, JP34, JP500, KU1, M11, MX1, TW18, VK, SP, FI, ID2, NL95, TW19, AP205, ϕkCb5, ϕkCb8r, ϕkCb12r, ϕkCb23r, 7s, and PRR1. Accordingly, in some embodiments, the aptamer is selected from binding proteins specifically binding any one of the adaptor proteins as described herein. In some embodiments, the aptamer sequence is a MS2 binding loop (5′-ggcccAACAUGAGGAUCACCCAUGUCUGCAGgggcc-3′ (SEQ ID NO: 70)). In some embodiments, the aptamer sequence is a QBeta binding loop (5′-ggcccAUGCUGUCUAAGACAGCAUgggcc-3′ (SEQ ID NO: 71)). In some embodiments, the aptamer sequence is a PP7 binding loop (5′-ggcccUAAGGGUUUAUAUGGAAACCCUUAgggcc-3′ (SEQ ID NO: 72)). A detailed description of aptamers can be found, e.g., in Nowak et al., “Guide RNA engineering for versatile Cas9 functionality,” Nucl. Acid. Res., 44(20):9555-9564, 2016; and WO 2016205764, which are incorporated herein by reference in their entirety.

The invention also encompasses methods for delivering multiple nucleic acid components, wherein each nucleic acid component is specific for a different target locus of interest thereby modifying multiple target loci of interest (for example, two different sgRNA each targeting a different target sequence within the same VEGFA mRNA may be employed in the construct of the invention). The nucleic acid component of the complex may comprise one or more protein-binding RNA aptamers. The one or more aptamers may be capable of binding a bacteriophage coat protein. The bacteriophage coat protein may be selected from the group comprising Qβ, F2, GA, fr, JP501, MS2, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. In certain embodiments, the bacteriophage coat protein is MS2.

### 5. Target RNA

The target RNA can be any RNA molecule of interest, including naturally-occurring and engineered RNA molecules. The target RNA can be an mRNA, a tRNA, a ribosomal RNA (rRNA), a microRNA (miRNA), an interfering RNA (siRNA), a ribozyme, a riboswitch, a satellite RNA, a microswitch, a microzyme, or a viral RNA.

In some embodiments, the target nucleic acid is associated with a condition or disease (e.g., an infectious disease or a cancer).

In certain embodiments, the target nucleic acid is an mRNA encoding VEGFA, such as human VEGFA.

In certain embodiments, the VEGFA mRNA target is any one of 17 known transcripts or isoforms produced by alternative promoter usage, alternative splicing, and/or alternative initiation.

In certain embodiments, the VEGFA mRNA target is any one of the following RefSeq number: NM_001025366.2 [P15692-14], NM_001025367.2 [P15692-16], NM_001025368.2 [P15692-11], NM_001025369.2 [P15692-17], NM_001025370.2 [P15692-12], NM_001033756.2 [P15692-15], NM_001171622.1 [P15692-18], NM_001171623.1 [P15692-1], NM_001171624.1 [P15692-2], NM_001171625.1 [P15692-3], NM_001171626.1 [P15692-4], NM_001171627.1 [P15692-5], NM_001171628.1 [P15692-9], NM_001171629.1 [P15692-8], NM_001171630.1 [P15692-10], NM_001204384.1 [P15692-6], NM_001204385.1 NM_001287044.1, NM_001317010.1, NM_003376.5 [P15692-13]. All nucleotide sequences are incorporated herein by reference.

Thus, in some embodiments, the systems described herein can be used to treat a condition or disease (such as wet AMD) by targeting these nucleic acids (e.g., VEGFA). For instance, the target nucleic acid associated with a condition or disease may be an RNA molecule that is overexpressed in a diseased cell (e.g., a disease cell in wetAMD patient eye). The target nucleic acid may also be a toxic RNA and/or a mutated RNA (e.g., an mRNA molecule having a splicing defect or a mutation). The target nucleic acid may also be an RNA that is specific for a particular microorganism (e.g., a pathogenic bacteria).

### 6. Complex and Cell

One aspect of the invention provides a complex of an engineered Class 2 type VI Cas13 protein, such as those substantially lacking collateral activity, such as CRISPR/Cas13e complex, comprising (1) any of the engineered Class 2 type VI Cas13 protein, such as those substantially lacking collateral activity (e.g., engineered Cas13e effector proteins, homologs, orthologs, fusions, derivative, conjugates, or functional fragments thereof as described herein), and (2) any of the guide RNA described herein, each including a spacer sequence designed to be at least partially complementary to a target RNA, and a DR sequence compatible with the engineered Class 2 type VI Cas13 protein, such as those substantially lacking collateral activity (e.g., Cas13e effector proteins), homologs, orthologs, fusions, derivatives, conjugates, or functional fragments thereof.

In certain embodiments, the complex further comprises the target RNA (such as a VEGFA mRNA) bound by the guide RNA.

In a related aspect, the invention also provides a cell comprising any of the complex of the invention. In certain embodiments, the cell is a prokaryote. In certain embodiments, the cell is a eukaryote.

### 7. Therapeutic Applications

The CRISPR systems described herein can have various therapeutic applications. Such applications may be based on one or more of the abilities below, both in vitro and in vivo, of the subject engineered Cas13, e.g., engineered CRISPR/Cas13e or Cas13f systems.

In some embodiments, the new engineered CRISPR systems can be used to treat various diseases and disorders, e.g., genetic disorders (e.g., monogenetic diseases), diseases that can be treated by nuclease activity (e.g., Pcsk9 targeting, Duchenne Muscular Dystrophy (DMD), BCL11a targeting), and various cancers, etc.

In some embodiments, the CRISPR systems described herein can be used to edit a target nucleic acid to modify the target nucleic acid (e.g., by inserting, deleting, or mutating one or more nucleic acid residues).

In one aspect, the CRISPR systems described herein can be used for treating a disease caused by overexpression of RNAs, toxic RNAs, and/or mutated RNAs (e.g., splicing defects or truncations). For example, expression of toxic RNAs may be associated with the formation of nuclear inclusions and late-onset degenerative changes in brain, heart, or skeletal muscle. In some embodiments, the disorder is myotonic dystrophy. In myotonic dystrophy, the main pathogenic effect of the toxic RNAs is to sequester binding proteins and compromise the regulation of alternative splicing (see, e.g., Osborne et al., “RNA-dominant diseases,” Hum. Mol. Genet., 2009 Apr. 15; 18(8):1471-81). Myotonic dystrophy (dystrophia myotonica (DM)) is of particular interest to geneticists because it produces an extremely wide range of clinical features. The classical form of DM, which is now called DM type 1 (DM1), is caused by an expansion of CTG repeats in the 3′-untranslated region (UTR) of DMPK, a gene encoding a cytosolic protein kinase. The CRISPR systems as described herein can target overexpressed RNA or toxic RNA, e.g., the DMPK gene or any of the mis-regulated alternative splicing in DM1 skeletal muscle, heart, or brain.

The CRISPR systems described herein can also target trans-acting mutations affecting RNA-dependent functions that cause various diseases such as, e.g., Prader Willi syndrome, Spinal muscular atrophy (SMA), and Dyskeratosis congenita. A list of diseases that can be treated using the CRISPR systems described herein is summarized in Cooper et al., “RNA and disease,” Cell, 136.4 (2009): 777-793, and WO 2016/205764 A1, both of which are incorporated herein by reference in the entirety. Those of skill in this field will understand how to use the new CRISPR systems to treat these diseases.

The CRISPR systems described herein can also be used in the treatment of various tauopathies, including, e.g., primary and secondary tauopathies, such as primary age-related tauopathy (PART)/Neurofibrillary tangle (NFT)-predominant senile dementia (with NFTs similar to those seen in Alzheimer Disease (AD), but without plaques), dementia pugilistica (chronic traumatic encephalopathy), and progressive supranuclear palsy. A useful list of tauopathies and methods of treating these diseases are described, e.g., in WO 2016205764, which is incorporated herein by reference in its entirety.

The CRISPR systems described herein can also be used to target mutations disrupting the cis-acting splicing codes that can cause splicing defects and diseases. These diseases include, e.g., motor neuron degenerative disease that results from deletion of the SMN1 gene (e.g., spinal muscular atrophy), Duchenne Muscular Dystrophy (DMD), frontotemporal dementia, and Parkinsonism linked to chromosome 17 (FTDP-17), and cystic fibrosis. The CRISPR systems described herein can further be used for antiviral activity, in particular against RNA viruses. The CRISPR-associated proteins can target the viral RNAs using suitable guide RNAs selected to target viral RNA sequences.

The CRISPR systems described herein can also be used to treat a cancer in a subject (e.g., a human subject). For example, the CRISPR-associated proteins described herein can be programmed with crRNA targeting a RNA molecule that is aberrant (e.g., comprises a point mutation or are alternatively-spliced) and found in cancer cells to induce cell death in the cancer cells (e.g., via apoptosis).

The CRISPR systems described herein can also be used to treat an autoimmune disease or disorder in a subject (e.g., a human subject). For example, the CRISPR-associated proteins described herein can be programmed with crRNA targeting a RNA molecule that is aberrant (e.g., comprises a point mutation or are alternatively-spliced) and found in cells responsible for causing the autoimmune disease or disorder.

Further, the CRISPR systems described herein can also be used to treat an infectious disease in a subject. For example, the CRISPR-associated proteins described herein can be programmed with crRNA targeting a RNA molecule expressed by an infectious agent (e.g., a bacteria, a virus, a parasite or a protozoan) in order to target and induce cell death in the infectious agent cell. The CRISPR systems may also be used to treat diseases where an intracellular infectious agent infects the cells of a host subject. By programming the CRISPR-associated protein to target a RNA molecule encoded by an infectious agent gene, cells infected with the infectious agent can be targeted and cell death induced.

Furthermore, in vitro RNA sensing assays can be used to detect specific RNA substrates. The CRISPR-associated proteins can be used for RNA-based sensing in living cells. Examples of applications are diagnostics by sensing of, for examples, disease-specific RNAs.

A detailed description of therapeutic applications of the CRISPR systems described herein can be found, e.g., in U.S. Pat. No. 8,795,965, EP 3009511, WO 2016205764, and WO 2017070605; each of which is incorporated herein by reference in its entirety.

In certain embodiments, the methods of the invention can be used to treat an eye disease or disorder. In some embodiments, the eye disease or disorder is amoebic keratitis, fungal keratitis, bacterial keratitis, viral keratitis, onchorcercal keratitis, keratoconjunctivitis, bacterial keratoconjunctivitis, viral keratoconjunctivitis, vernal keratoconjunctivitis, atopic keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, noninfectious uveitis, anterior uveitis, posterior uveitis (including toxoplasmosis), pan-uveitis, an inflammatory disease of the vitreous or retina, endophthalmitis prophylaxis and treatment, macular edema, macular degeneration, wet age related macular degeneration (wet AMD), dry age related macular degeneration (dry AMD), diabatic macular edema (DME), allergic conjunctivitis, proliferative and non-proliferative diabetic retinopathy, hypertensive retinopathy, an autoimmune disease of the retina, primary and metastatic intraocular melanoma, other intraocular metastatic tumors, open angle glaucoma, Stargardt's disease, Fundus Flavimaculatus, closed angle glaucoma, pigmentary glaucoma, retinitis pigmentosa (RP), Leber's congenital amaurosis (LCA), Usher's syndrome, choroideremia, a rod-cone or cone-rod dystrophy, a ciliopathy, a mitochondrial disorder, progressive retinal atrophy, a degenerative retinal disease, geographic atrophy, a familial or acquired maculopathy, a retinal photoreceptor disease, a retinal pigment epithelial-based disease, cystoid macular edema, retinal detachment, traumatic retinal injury, iatrogenic retinal injury, macular holes, macular telangiectasia, a ganglion cell disease, an optic nerve cell disease, optic neuropathy, ischemic retinal disease, retinopathy of prematurity, retinal vascular occlusion, familial macroaneurysm, a retinal vascular disease, an ocular vascular diseases, a vascular disease, an ischemic optic neuropathy disease, diabetic retinal oedema, senile macular degeneration due to sub-retinal neovascularization, myopic retinopathy, retinal ischemia, choroidal vascular insufficiency, choroidal thrombosis and neovascular retinopathies resulting from carotoid artery ischemia, corneal neovascularisation, a corneal disease or opacification with an exudative or inflammatory component, diffuse lamellar keratitis, neovascularisation due to penetration of the eye or contusive ocular injury, rubosis iritis, Fuchs' heterochromic iridocyclitis, chronic uveitis, anterior uveitis, inflammatory conditions resulting from surgeries such as LASIK, LASEK, refractive surgery, IOL implantation; irreversible corneal oedema as a complication of cataract surgery, oedema as a result of insult or trauma, inflammation, infectious and non-infectious conjunctivitis, iridocyclitis, iritis, scleritis, episcleritis, superficial punctuate keratitis, keratoconus, posterior polymorphous dystrophy, Fuch's dystrophies, aphakic and pseudophakic bullous keratopathy, corneal oedema, scleral disease, ocular cicatrcial pemphigoid, pars planitis, Posner Schlossman syndrome, Behcet's disease, Vogt-Koyanagi-Harada syndrome, hypersensitivity reactions, ocular surface disorders, conjunctival oedema, Toxoplasmosis chorioretinitis, inflammatory pseudotumor of the orbit, chemosis, conjunctival venous congestion, periorbiatal cellulits, acute dacroycystitis, non-specific vasculitis, sarcoidosis, cytomegalovirus infection, and combinations thereof. In certain embodiments, the eye disease or disorder is age related macular degeneration. In some embodiments, the eye disease or disorder is wet age related macular degeneration (wet AMD) or dry age related macular degeneration (dry AMD). In some embodiments, the eye disease or disorder is wet age related macular degeneration (wet AMD).

In certain embodiments, the methods of the invention can be used to treat a neurodegenerative disease or disorder. In some embodiments, the neurodegenerative disease or disorder is alcoholism, Alexander's disease, Alper's disease, Alzheimer's Disease, amyotrophic lateral sclerosis (ALS), ataxia telangiectasia, neuronal ceroid lipofuscinoses, Batten disease, bovine spongiform encephalopathy (BSE), Canavan disease, cerebral palsy, Cockayne syndrome, corticobasal degeneration, Creutzfeldt-Jakob disease, frontotemporal lobar degeneration, Huntington's disease, HIV-associated dementia, Kennedy's disease, Lewy body dementia, neuroborreliosis, primary age-related tauopathy (PART)/Neurofibrillary tangle-predominant senile dementia, Machado-Joseph disease, multiple system atrophy, multiple sclerosis, multiple sulfatase deficiency, mucolipidoses, narcolepsy, Niemann Pick disease, Parkinson's Disease, Pick's disease, Pompe disease, primary lateral sclerosis, prion diseases, neuronal loss, cognitive defect, motor neuron diseases, Duchenne Muscular Dystrophy (DMD), frontotemporal dementia, frontotemporal dementia and parkinsonism linked to chromosome 17, Lytico-Bodig disease (Parkinson-dementia complex of Guam), neuroaxonal dystrophies, Refsum's disease, Schilder's disease, subacute combined degeneration of spinal cord secondary to pernicious anaemia, Spielmeyer-Vogt-Sjogren-Batten disease, Parkinsonism linked to chromosome 17 (FTDP-17), Prader Willi syndrome, Myotonic dystrophy, chronic traumatic encephalopathy including dementia pugilistica, spinocerebellar ataxia, spinal muscular atrophy, Steele-Richardson-Olszewski disease, Tabes dorsalis, Niemann-Pick Type C (NPC1 and/or NPC2 defect), Smith-Lemli-Opitz Syndrome (SLOS), an inborn error of cholesterol synthesis, Tangier disease, Pelizaeus-Merzbacher disease, a neuronal ceroid lipofuscinosis, a primary glycosphingolipidosis, Farber disease or multiple sulphatase deficiency, Gaucher disease, Fabry disease, GM1 gangliosidosis, GM2 gangliosidosis, Krabbe disease, metachromatic leukodystrophy (MLD), NPC, GM1 gangliosidosis, Fabry disease, a neurodegenerative mucopolysaccharidosis, MPS I, MPS IH, MPS IS, MPS II, MPS III, MPS IIIA, MPS IIIB, MPS IIIC, MPS HID, MPS, IV, MPS IV A, MPS IV B, MPS VI, MPS VII, MPS IX, a disease with secondary lysosomal involvement, SLOS, Tangier disease, ganglioglioma, gangliocytoma, meningioangiomatosis, postencephalitic parkinsonism, subacute sclerosing panencephalitis, lead encephalopathy, tuberous sclerosis, Hallervorden-Spatz disease, lipofuscinosis, cerebellar ataxia, parkinsonism, Louis-Barr syndrome, multiple systems atrophy, fronto-temporal dementia or lower body Parkinson's syndrome, Niemann Pick disease, Niemann Pick type C, Niemann Pick type A, Tay-Sachs disease, multisystemic atrophy cerebellar type (MSA-C), fronto-temporal dementia with parkinsonism, progressive supranuclear palsy, cerebellar downbeat nystagmus, Sandhoff's disease or mucolipidosis type II, or combinations thereof.

In some embodiments, the neurodegenerative disease or disorder is Alzheimer's Disease, amyotrophic lateral sclerosis (ALS), ataxia telangiectasia, cerebral palsy, Cockayne syndrome, corticobasal degeneration, Creutzfeldt-Jakob disease, frontotemporal lobar degeneration, Huntington's disease, Lewy body dementia, multiple sclerosis, Parkinson's Disease, Pick's disease, Pompe disease, Duchenne Muscular Dystrophy (DMD), Prader Willi syndrome, spinal muscular atrophy, or combinations thereof.

In certain embodiments, the methods of the invention can be used to treat a cancer. As used herein, “cancer” refers to all types of cancer or neoplasm or malignant tumors including leukemias, carcinomas and sarcomas, whether new or recurring. Specific examples of cancers are: carcinomas, sarcomas, myelomas, leukemias, lymphomas and mixed type tumors. Non-limiting examples of cancers that may treated by methods and compositions described herein include, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; plasmacytoma, colorectal cancer, rectal cancer, and hairy cell leukemia.

Non-limiting examples of leukemia diseases include, acute nonlymphocytic leukemia, chronic lymphocytic leukemia, acute granulocytic leukemia, chronic granulocytic leukemia, acute promyelocytic leukemia, adult T-cell leukemia, a leukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

Non-limiting exemplary types of carcinomas include, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, merkel cell carcinoma, salivary gland carcinoma and carcinoma scroti.

Sarcomas include, but are not limited to, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

In certain embodiments, the methods of the invention can be used to introduce the CRISPR systems described herein into a cell, and cause the cell and/or its progeny to alter the production of one or more cellular produces, such as growth factor (such as VEGFA), antibody, starch, ethanol, or any other desired products. Such cells and progenies thereof are within the scope of the invention.

In certain embodiments, the methods and/or the CRISPR systems described herein lead to modification of the translation and/or transcription of one or more RNA products of the cells. For example, the modification may lead to increased transcription/translation/expression of the RNA product. In other embodiments, the modification may lead to decreased transcription/translation/expression of the RNA product.

In certain embodiments, the cell is a prokaryotic cell.

In certain embodiments, the cell is a eukaryotic cell, such as a mammalian cell, including a human cell (a primary human cell or an established human cell line). In certain embodiments, the cell is a non-human mammalian cell, such as a cell from a non-human primate (e.g., monkey), a cow/bull/cattle, sheep, goat, pig, horse, dog, cat, rodent (such as rabbit, mouse, rat, hamster, etc). In certain embodiments, the cell is from fish (such as salmon), bird (such as poultry bird, including chick, duck, goose), reptile, shellfish (e.g., oyster, claim, lobster, shrimp), insect, worm, yeast, etc. In certain embodiments, the cell is from a plant, such as monocot or dicot. In certain embodiment, the plant is a food crop such as barley, cassava, cotton, groundnuts or peanuts, maize, millet, oil palm fruit, potatoes, pulses, rapeseed or canola, rice, rye, sorghum, soybeans, sugar cane, sugar beets, sunflower, and wheat. In certain embodiment, the plant is a cereal (barley, maize, millet, rice, rye, sorghum, and wheat). In certain embodiment, the plant is a tuber (cassava and potatoes). In certain embodiment, the plant is a sugar crop (sugar beets and sugar cane). In certain embodiment, the plant is an oil-bearing crop (soybeans, groundnuts or peanuts, rapeseed or canola, sunflower, and oil palm fruit). In certain embodiment, the plant is a fiber crop (cotton). In certain embodiment, the plant is a tree (such as a peach or a nectarine tree, an apple or pear tree, a nut tree such as almond or walnut or pistachio tree, or a citrus tree, e.g., orange, grapefruit or lemon tree), a grass, a vegetable, a fruit, or an algae. In certain embodiment, the plant is a nightshade plant; a plant of the genus Brassica; a plant of the genus Lactuca; a plant of the genus Spinacia; a plant of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

A related aspect provides cells or progenies thereof modified by the methods of the invention using the CRISPR systems described herein.

In certain embodiments, the cell is modified in vitro, in vivo, or ex vivo. In certain embodiments, the cell is a stem cell.

### 8. Delivery

Through this disclosure and the knowledge in the art, the CRISPR systems described herein comprising an engineered Class 2 type VI Cas13 protein, such as those substantially lacking collateral activity (such as Cas13e or Cas13f, e.g., Cas13X.1), or any of the components thereof described herein (Cas13 proteins, derivatives, functional fragments or the various fusions or adducts thereof, and guide RNA/crRNA), nucleic acid molecules thereof, and/or nucleic acid molecules encoding or providing components thereof, can be delivered by various delivery systems such as vectors, e.g., plasmids and viral delivery vectors, using any suitable means in the art. Such methods include (and are not limited to) electroporation, lipofection, microinjection, transfection, sonication, gene gun, etc.

In certain embodiments, the CRISPR-associated proteins and/or any of the RNAs (e.g., guide RNAs or crRNAs) and/or accessory proteins can be delivered using suitable vectors, e.g., plasmids or viral vectors, such as adeno-associated viruses (AAV), lentiviruses, adenoviruses, retroviral vectors, and other viral vectors, or combinations thereof. The proteins and one or more crRNAs can be packaged into one or more vectors, e.g., plasmids or viral vectors. For bacterial applications, the nucleic acids encoding any of the components of the CRISPR systems described herein can be delivered to the bacteria using a phage. Exemplary phages, include, but are not limited to, T4 phage, Mu, λ phage, T5 phage, T7 phage, T3 phage, Φ29, M13, MS2, Qβ, and ΦX174.

In certain embodiments, the delivery is through AAV9 serotype viral vectors, such as AAV9 or other Clade F capsids, or mutants/derivatives based on AAV9 (e.g, sharing significant sequence homology and spectrum of tropism as AAV9).

In some embodiments, the vectors, e.g., plasmids or viral vectors (e.g., AAV viral vectors), are delivered to the tissue of interest by, e.g., intramuscular injection, intravenous administration, transdermal administration, intranasal administration, oral administration, or mucosal administration.

In certain embodiments, the AAV viral particle of the invention (e.g., AAV9 viral particle) is delivered through subretinal injection, such as subretinal injection following a vitrectomy. In certain embodiments, the delivery is one subretinal injection per eye. For example, under adequate anesthesia, a subretinal injection of a therapeutically effective amount of the vector genomes (vg) of the invention in a suitable total volume (e.g., about 0.1-0.5 mL, such as 0.3 mL) is performed on each human eye separately, using standard vitreoretinal techniques for subretinal surgery. In certain embodiments, the subject is given a short-term corticosteroid regimen of oral prednisone (or the equivalent), before and/or after the subretinal injection to each eye in need to treatment.

Delivery may be either via a single dose, or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adenoviruses, which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. The delivery methods and the doses are described, e.g., in WO 2016205764 A1 and U.S. Pat. No. 8,454,972 B2, both of which are incorporated herein by reference in the entirety.

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter, (ii) a sequence encoding a nucleic acid-targeting CRISPR-associated proteins and/or an accessory protein, each operably linked to a promoter (e.g., the same promoter or a different promoter); (iii) a selectable marker, (iv) an origin of replication; and (v) a transcription terminator downstream of and operably linked to (ii). The plasmids can also encode the RNA components of a CRISPR complex, but one or more of these may instead be encoded on different vectors. The frequency of administration is within the ambit of the medical or veterinary practitioner (e.g., physician, veterinarian), or a person skilled in the art.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

Further means of introducing one or more components of the new CRISPR systems to the cell is by using cell penetrating peptides (CPP). In some embodiments, a cell penetrating peptide is linked to the CRISPR-associated proteins. In some embodiments, the CRISPR-associated proteins and/or guide RNAs are coupled to one or more CPPs to effectively transport them inside cells (e.g., plant protoplasts). In some embodiments, the CRISPR-associated proteins and/or guide RNA(s) are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin β3 signal peptide sequence, polyarginine peptide Args sequence, Guanine rich-molecular transporters, and sweet arrow peptide. CPPs and methods of using them are described, e.g., in Hallbrink et al., “Prediction of cell-penetrating peptides,” Methods Mol. Biol., 2015; 1324:39-58; Ramakrishna et al., “Gene disruption by cell-penetrating peptide-mediated delivery of Cas9 protein and guide RNA,” Genome Res., 2014 June; 24(6):1020-7; and WO 2016205764 A1; each of which is incorporated herein by reference in its entirety.

Various delivery methods for the CRISPR systems described herein are also described, e.g., in U.S. Pat. No. 8,795,965, EP 3009511, WO 2016205764, and WO 2017070605; each of which is incorporated herein by reference in its entirety.

### 9. Kits

Another aspect of the invention provides a kit, comprising any two or more components of the subject CRISPR/Cas system described herein comprising an engineered Class 2 type VI Cas13 protein, such as those substantially lacking collateral activity, such as the Cas13X.1, Cas13e and Cas13f proteins, derivatives, functional fragments or the various fusions or adducts thereof, guide RNA/crRNA, complexes thereof, vectors encompassing the same, or host encompassing the same.

In certain embodiments, the kit comprises an rAAV vector genome or an rAAV viral particle described herein comprising an polynucleotide comprising a Cas13X coding sequence, as well as coding sequence for one or more sgRNA targeting VEGFA separated by DR sequences (such as SEQ ID NO: 6).

In certain embodiments, the kit further comprise an instruction to use the components encompassed therein, and/or instructions for combining with additional components that may be available elsewhere.

In certain embodiments, the kit further comprise one or more nucleotides, such as nucleotide(s) corresponding to those useful to insert the guide RNA coding sequence into a vector and operably linking the coding sequence to one or more control elements of the vector.

In certain embodiments, the kit further comprise one or more buffers that may be used to dissolve any of the components, and/or to provide suitable reaction conditions for one or more of the components. Such buffers may include one or more of PBS, HEPES, Tris, MOPS, Na2CO3, NaHCO₃, NaB, or combinations thereof. In certain embodiments, the reaction condition includes a proper pH, such as a basic pH. In certain embodiments, the pH is between 7-10.

In certain embodiments, any one or more of the kit components may be stored in a suitable container.

### 10. Host Cells and AAV Production

General principles of rAAV production are known in the art. See review in, for example, Carter (Current Opinions in Biotechnology, 1533-539, 1992); and Muzyczka, Curr. Topics in Microbial, and Immunol 158:97-129, 1992, both incorporated herein by reference). Various approaches are described in Ratschin et al (Mol. Cell. Biol. 4:2072, 1984; Hermonat et al. (Proc. Natl. Acad. Sci. USA 81:6466, 1984); Tratschin et al. (Mol. Cell. Biol. 5:3251, 1985); McLaughlin et al. (J. Virol 62:1963, 1988); and Lebkowski et al. (Mol. Cell. Biol 7:349, 1988), Samulski et al. (J. Virol 63:3822-3828, 1989); U.S. Pat. No. 5,173,414; WO 95/13365 and U.S. Pat. No. 5,658,776; WO 95/13392; WO 96/17947; PCT/US98/18600; WO 97/09441; WO 97/08298; WO 97/21825; WO 97/06243; WO 99/11764; Perrin et al. (Vaccine 13:1244-1250, 1995; Paul et al. (Human Gene Therapy 4:609-615, 1993); Clark et al. (Gene Therapy 3:1124-1132, 1996; U.S. Pat. Nos. 5,786,211; 5,871,982; and 6,258,595.

AAV vector serotypes can be matched to target cell types. For example, Table 2 of WO2018002719A1 lists exemplary cell types that can be transduced by the indicated AAV serotypes (incorporated herein by reference).

Packaging cells are used to form virus particles that are capable of infecting a host cell. Such cells include HEK293 and Sf9 cells, which can be used to package AAV and adenovirus.

Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions can be supplied in trans by the packaging cell line, usually as a result of expression of these viral functions/proteins (such as the rep and cap genes for AAV) either as transgenes integrated into the packaging cell, or as transgenes on a second viral vector or expression vector introduced into the packaging cell.

For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a gene of interest) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some embodiments, the subject rAAV viral particle is produced using a baculovirus expression system packaged in insect cells such as Sf9 cells. See, for example, WO2007046703, WO2007148971, WO2009014445, WO2009104964, WO2013036118, WO2011112089, WO2016083560, WO2015137802, and WO2019016349, all incorporated herein by reference.

The vector titers are usually expressed as viral genomes per ml (vg/ml). In certain embodiments, viral titers is above 1×10⁹, above 5×10¹⁰, above 1×10¹¹, above 5×10¹¹, above 1×10¹², above 5×10¹², or above 1×10¹³ vg/ml.

### 11. Non-Human Primate Model of Wet AMD/CNV

Another aspect of the invention provides a non-human primate (NHP) model of wet AMD/choroidal neovascularization (CNV), and method of making and using the same.

NHP models may be useful, since rodent models may not be appropriate to test the effects of certain wetAMD treatments, such as humanized anti-VEGF antibody (including the current standard of care for neovascular AMD, the anti-VEGF antibody ranibizumab and bevaciumab), presumably due to differences between the rodent and human forms of VEGF, whereas the studies in macaques showed strong evidence of safety and efficacy and helped to provide the basis for human clinical trials.

While not wishing to be bound by any particular theory, it is believed that administering immunosuppressor to an NHP before laser induction of CNV resulted in longer lasting laser-induced CNV, e.g., to more than 2-4 weeks. In contrast, previous NHP model of laser-induced CNV lasts about 2-4 weeks.

Thus in one aspect, the invention provides a NHP (such as Cynomolgus Monkeys (Macaca fascicularis)) model of wet AMD/CNV, comprising a NHP (such as Cynomolgus Monkeys (Macaca fascicularis)) with an eye having developed laser-induced CNV, wherein the CNV is induced by laser photocoagulation about one month (e.g., about 3 weeks, 4 weeks, 31 days, or 5 weeks) after first administering one or more immunosuppressors to the NHP, and the laser-induced CNV persists at least about 4 weeks, e.g., at least 5 weeks, at least 6 weeks, at least 7 weeks, at least 8 weeks, at least 9 weeks, or at least 10 weeks.

In some embodiments, the one or more immunosuppressors are first administered at least 2 weeks, at least 3 weeks, at least 4 weeks, at least 5 weeks, at least 6 weeks, at least 7 weeks, or at least 8 weeks prior to laser photocoagulation.

In certain embodiments, the one or more immunosuppressors are administered daily, for at least 20 days, at least 22 days, at least 24 days, at least 26 days, at least 28 days, at least 30 days, at least 32 days, at least 34 days, at least 36 days, at least 38 days, at least 40 days, at least 42 days, at least 43 days, at least 44 days, at least 46 days, at least 48 days, or at least 50 days.

In some embodiments, laser photocoagulation-induced CNV persists for at least 4 weeks, at least 5 weeks, at least 6 weeks, at least 7 weeks, at least 8 weeks, at least 9 weeks, or at least 10 weeks.

In certain embodiments, the one or more immunosuppressors comprise triptolide, corticosteroids such as prednisone, calcineurin inhibitors such as tacrolimus (ENVARSUS XR® or Protopic), cyclosporine (GENGRAF®, NEORAL® or SANDIMMUNE®), Inosine monophosphate dehydrogenase (IMDH) inhibitors such as mycophenolate mofetil (CELLCEPT®), imuran (Azathioprine), mechanistic target of rapamycin (mTOR) inhibitors such as sirolimus (RAPAMUNE®), Janus kinase inhibitors such as tofacitinib (XEUANZ®), monoclonal antibodies such as basiliximab (SIMULECT®).

In certain embodiments, the immunosuppressants comprise calcineurin inhibitors, interleukin inhibitors, and/or selective immunosuppressants and TNF alfa inhibitors (such as adalimumab, infliximab, certolizumab pegol, golimumab, and other anti-TNFα neutralizing antibodies or fusion proteins such as etanercept).

In certain embodiments, the laser photocoagulation is conducted in the perimacular region of the eye, at about 1.5-2 disk diameter from the foveal center.

In certain embodiments, laser photocoagulation comprises a setting of about 50 μm spot size, about 0.1 second's (or 100 ms) duration, and/or about 400-700 mW intensity.

In certain embodiments, photocoagulation is repeated to produce a break in Bruch's membrane and bubble formation.

In certain embodiments, the laser is argon laser.

In certain embodiments, the NHP is Cynomolgus Monkeys (Macaca fascicularis), stumptailed macaque (Macaca speciosa), rhesus macaques (Macaca mulatta), or African green monkey (Chlorocebus sabaeus).

In certain embodiments, the NHP is Cynomolgus Monkeys (Macaca fascicularis).

Another aspect of the invention provides a method of identifying an inhibitor of wet AMD/CNV development or progression in the subject NHP model of wet AMD/CNV, the method comprising contacting the retina of the NHP model of wet AMD/CNV with a candidate inhibitor, and determining the extent said candidate inhibitor inhibits the progression of CNV as compared to a vehicle control, wherein the candidate inhibitor that statistically significantly inhibits CNV progression compared to the vehicle control is selected as the inhibitor of wet AMD/CNV.

In certain embodiments, the candidate inhibitor comes into contact with the retina via subretinal injection.

In certain embodiments, the candidate inhibitor comes into contact with the retina through a canular inserted through a punctuation spot on the eye of the NHP.

In certain embodiments, the candidate inhibitor comes into contact with the retina after first administering one or more immunosuppressors to the NHP.

In certain embodiments, the candidate inhibitor comes into contact with the retina 1, 2, 3, 4, or 5 days after first administering one or more immunosuppressors to the NHP.

In certain embodiments, the candidate inhibitor comes into contact with the retina before (e.g., 3, 4, or 5 weeks before) laser induction of CNV.

In certain embodiments, the candidate inhibitor comprises an AAV viral vector.

### EXAMPLES

### Example 1 High Efficiency In Vitro Knockdown of VEGFA Expression by Engineered Cas13X.1 Having Reduced Collateral Effect

This example demonstrates the high in vitro knockdown efficiency of VEGFA expression by the subject Cas13X.1 construct—hfCas13X.1-sg^{VEGFA} editing box. Briefly, plasmids expressing hfCas13X.1 and sg^{VEGFA} and control plasmids (FIGS. 3A-3C) were respectively transient transfected into cultured 293T cells and harvested 48 hours later.

The hfCas13X.1-sg^{VEGFA} construct in FIG. 3B encodes the Cas13X.1 coding sequence, as well as two sgRNA targeting VEGFA. A third coding sequence encoding the mCherry reporter under the transcriptional control of the CMV promoter was also included. The negative control construct in FIG. 3A only had the mCherry reporter driven by the CMV promoter. Another control in FIG. 3C expressed shRNA against VEGFA, under the transcriptional control of the same U6 promoter used to drive sgRNA expression in the hfCas13X.1-sg^{VEGFA} construct.

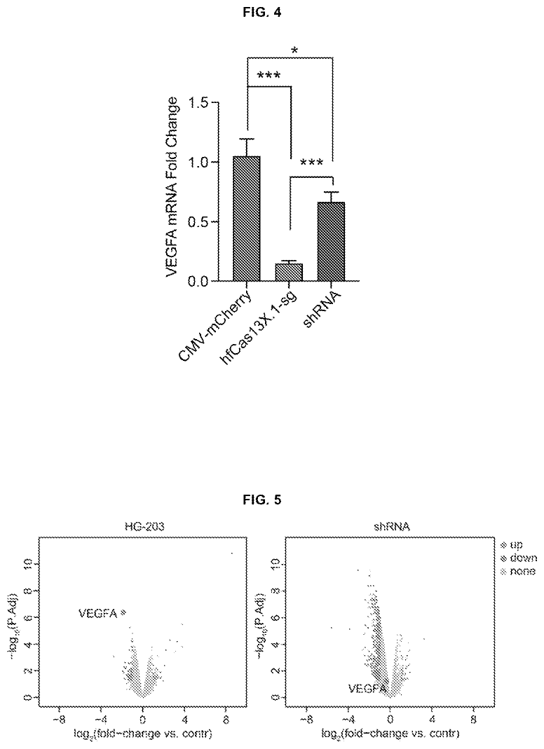

RNA were extracted from the transfected cells, and expression levels of VEGFA were measured by RT-PCR. Knockdown of VEGFA expression was 84.76±1.89% by hfCas13X.1-sg^{VEGFA} editing box, and only ˜32.99±7.9% by shRNA, indicating great potential for wetAMD therapy (FIG. 4).

Off-target RNA detection by hfCas13X.1-sg^{VEGFA} in comparison to VEGFA shRNA was also determined, and the results were shown in a volcano graph (FIG. 5).

### Example 2 High Efficiency In Vivo Knockdown of VEGFA Expression by Engineered Cas13X.1 Having Reduced Collateral Effect

To demonstrate the in vivo editing efficiency of the subject Cas13X.1 constructs, AAV9-hfCas13X.1-sg^{VEGFA} with different doses (range between 1×10⁶ (or 1E+6) and 1×10¹⁰ (or 1E+10) vg/eye) were injected into the eyes of C57BL/6 mice subretinally. The AAV vector genome having the same sequence elements as that used in Example 1, encapsidated within an AAV9 capsid shell, was used in this experiment.

Injected eyes were harvested 8-14 week post dosing. Retina RNA was extracted, and RNA expression level of hfCas13X.1, sg^{VEGFA} and VEGFA were detected by qPCR.

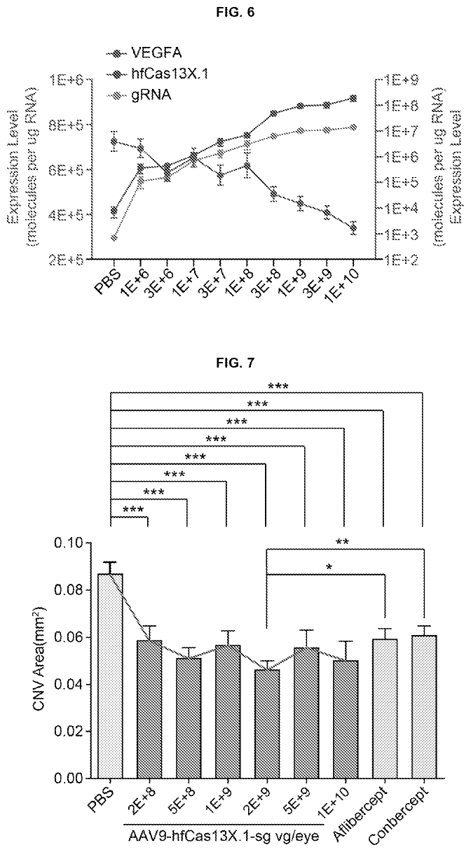

Expression level of hfCas13X.1 and sg^{VEGFA} ranges from 1E+2 to 1E+9 copies per μg RNA, and significant correlated with in vivo knock down efficiency of VEGFA (FIG. 6).

Laser induced choroidal neovascularization (CNV) mouse and non-human primate animal model were used for in vivo CNV growth inhibition study. Vehicle and AAV9-hfCas13X.1-sg^{VEGFA} with different doses (range from 2E+8 to 1E+10 vg/eye) were injected subretinally into the mouse eyes. 4 weeks later, CNVs were induced by laser irradiation and therapeutic effect were assessed 7 days later.

The results showed that CNV growth was significantly inhibited by AAV9-hfCas13X.1-sg^{VEGFA}, with the lowest effective doses of about 2E+8 vg/eye, and the maximum inhibition rate reached ˜46.6±4.55%. This is statistically significantly better than that of the marketed VEGFA antagonistic products, aflibercept (˜31.45±4.08%) and conbercept (˜29.63±3.63%) (FIG. 7).

Aflibercept is a soluble decoy receptor that binds VEGF-A, VEGF-B and placental growth factor (PIGF) with a greater affinity than the native VEGF receptors. Aflibercept competes with VEGF receptor for binding to VEGFA, thus reducing VEGFA signaling.

Conbercept, sold under the commercial name Lumitin, is an anti-VEGF antibody approved by the China State FDA (CFDA) for treating neovascular age-related macular degeneration (AMD) and diabetic macular edema (DME).

Considering both effectiveness and safety, 2E+11 vg/mL was chosen as the dosage used on NHP (non-human primate) study. About 100 μL of 2E+11 vg/ml AAV9-hfCas13X.1-sg^{VEGFA} were subretinally injected into eyes of NHP (pre-screened as AAV9 neutralizing antibody negative), and laser was then used to induce CNV 4 weeks later. Growth of CNV were measured with CNV area and height of SHRM (subretinal hyper-reflective material) at 1/2/4/6/9/14/19 weeks post laser.

It was found that AAV9-hfCas13X.1-sgVEGFA inhibited the growth of CNV both in area and SHRM height, and the inhibition effect lasted for more than 4 months.

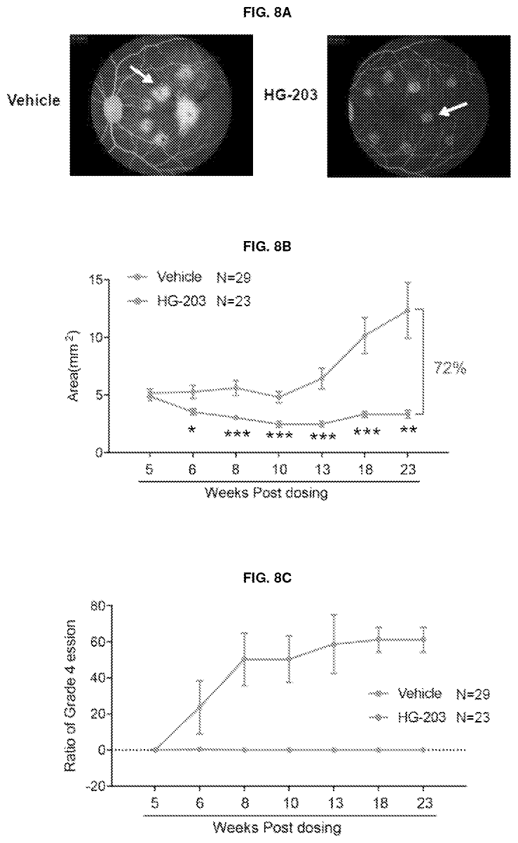

CNV growth, as determined by fundus fluorescein angiography, showed that hfCas13X.1-sgVEGFA inhibited growth of CNV in comparison to an untreated eye (FIG. 8A). The area of CVN was diminished by 72% in comparison to untreated eyes after 23 weeks post treatment (FIG. 8B). Ratio of Grade 4 lesions in untreated eyes increased to about 61% while eyes treated with hfCas13X.1-sgVEGFA were at 0%.

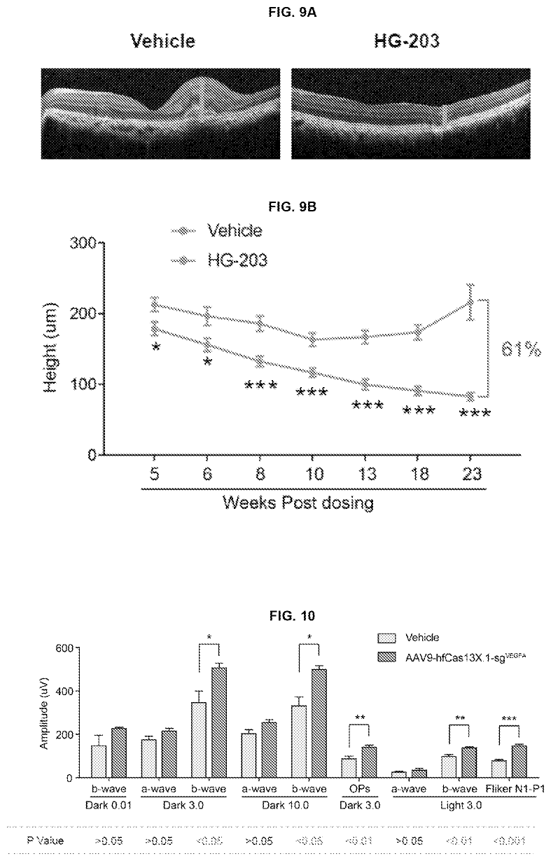

CNV SHRM (subretinal hyper-reflective material) height was measured in optical coherent tomography (OCT) treated with hfCas13X.1-sg^{VEGFA} versus untreated eyes (FIG. 9A). CNV height was decreased by hfCas13X.1-sg^{VEGFA} by about 61% after 23 weeks post dosing in comparison to untreated eyes (FIG. 9B).

To further assess the effect of AAV9-hfCas13X.1I-sg^{VEGFA} on wetAMD, visual function of NHP CNV model were tested by with ERG and AAV9-hfCas13X.1I-sg^{VEGFA} treated eyes showed much better vision than the vehicle treated eyes in both rod function and cone function (FIG. 10).

In summary, the data presented herein demonstrated that the hfCas13X.1I-sg^{VEGFA} editing box efficiently reduced expression of VEGFA in cultured 293T cells and in mouse eyes and subretinally injection of AAV9-hfCas13X.1-sg^{VEGFA} could inhibit laser induced CNV growth, both in mice and NHP. The results demonstrated the ability and potential of AAV9-hfCas13X.1-sg^{VEGFA} on therapy of wetAMD in human and also other VEGFA related macular degeneration diseases.

### Methods

### Animals

C57BL/6J animals were purchased from Beijing Vital River Laboratory Animal Technology Co., Ltd. and housed in the in-house animal facility on 12h:12h light/dark cycle with food and water ad libitum. All experimental protocols were approved by the Animal Care and Use Committee.

Cynomolgus Monkeys (Macaca fascicularis), 2-3 years of age and 2.0-3.5 kg of weight, were used in this study and were originally obtained from GuangDong Blooming-Spring Biological Technology Development Co., Ltd. Animals will be socially housed (up to 3 animals of same sex and same dosing group together) in stainless steel cages of rooms on a 12-hour light/dark cycle. IACUC applications relating to the protocol and any amendments or procedures involving the care and use of animals on this study is reviewed and approved by Testing Facility Institutional Animal Care and Use Committee (IACUC) prior to the initiation of such procedures. A staff veterinarian will monitor the study for animal welfare issues and consult with the Study Director to determine appropriate treatment.

### AAV Vector Preparation

Recombinant AAV9 viral particles were generated by triple transfection of 293T cells using polyethylenimine (PEI). Viral particles were harvested from the media at 72 hours post transfection and from the cells and media at 120 hours. Cell pellets were resuspended in 10 mM Tris with 10 mM MgCl₂ and 150 mM sodium chloride, pH 7.6, freeze-thawed three times, and treated with 125 U/mL Benzonase (Sigma) at 37° C. for at least 1 hr. Viral media was concentrated by precipitation with 10% polyethylene glycol 8000 (Sigma-Aldrich) with 625 mM sodium chloride, resuspended in PBS with 0.001% Pluronic™ F-68 Non-ionic Surfactant, and then added to the lysates. The combined stocks were then adjusted to 1000 mM NaCl, incubated at 37° C. for 1 hr, and clarified by centrifugation at 2000 g. The clarified stocks were then purified over iodixanol (Optiprep, Sigma; D1556) step gradients (15%, 25%, 40% and 58%) Viruses were concentrated and formulated in PBS with 0.001% Pluronic™ F-68 Non-ionic Surfactant. Virus titers were determined by measuring the number of DNasel resistant vector genomes using qPCR with linearized genome plasmid as a standard.

### VEGFA Knockdown in Cell Culture

293T cells were cultured in Dulbecco's modified eagle medium (DMEM) containing 10% fetal bovine serum (FBS) and penicillin/streptomycin, and maintained at 37° C. with 5% CO₂. Cells were seeded in 6-well plates (1.0-1.2E+6 cells/well) and were transfected with 4ug vectors expressing hfCas13X.1-sg^{VEGFA} and mCherry using PEI. Control plasmids expressing mCherry only or shRNA and mCherry. mCherry positive cells were isolated using flow cytometry 2 days after transfection. Total RNA was first purified using Trizol (Ambion) and then transcribed into complementary DNA (HiScript Q RT SuperMix for qPCR, Vazyme, Biotech). qPCR reactions were tracked by SYBR green probe (AceQ qPCR SYBR Green Master Mix, Vazyme, Biotech). VEGFA qPCR primers are: Forward, 5′-GAGGGCAGAATCATCACGAAG-3′ (SEQ ID NO: 73); Reverse, 5′-GTGAGGTITGATCCGCATAATC-3′ (SEQ ID NO: 74), 18s RNA qPCR primers are: Forward, 5′-TTGGTGGAGCGATITGTCTG-3′ (SEQ ID NO: 75); Reverse, 5′-GAATGGGGTTCAACGGGTTA-3′(SEQ ID NO: 76). shRNA1: -GTGCTGTAGGAAGCTCATCTCTCCTAT-(SEQ ID NO: 77); shRNA2: -GAAGATGTCCACCAGGGTCT-(SEQ ID NO: 78).

### Subretinal Injection

Mice—8 weeks old mice were anesthetized with mixture of zoletil (60 μg/g) and xylazine (10 μg/g). A small hole at slight posterior to the limbus were punctured with a sterile 31 G 1/2 needle following pupil dilation. 1 μL AAV9-hfCas13X.1-sg^{VEGFA} with different dose, antibody (Aflibercept 2.5 μg/μL, Conbercept 0.625 μg/μL) or vehicle were subretinally injected through the hole using a Hamilton syringe with a 33G blunt needle.

NHP—Both eyes of the monkeys were instilled with 1-2 drops of tropicamide eye drops to dilute the pupils. Thereafter, the animals were sedated with 10-30 mg/kg ketamine intramuscular injection, and anesthetized with xylazine intramuscular injection at a dosage of 0.5˜0.75 mg/kg. Animals were laid on the operating table, eyelid were opened with eye speculum to expose the eyeball. Adjust the until fundus is clearly visible. The eyeball position was fixed with forceps after fundus was clearly visible through stereo microscope. The eyeball wall was punctured at 3-4 mm posterior to limbus avoiding blood vessels and other ocular tissue. Canular were inserted through the punctuation spot, and then cannula tip was placed in contact with the retina surface, pushed forward into retina. AAV9-hfCas13X.1-sg^{VEGFA} or vehicle were injected slowly and the injected retinal would be uplifted. Then the cannula was pulled out from the retina but remained in the vitreous body for at least 30 seconds before fully pulled out from the eyeball. Injected animals were taken care by keeping warm with a blanket till they can move freely.

### VEGFA Knockdown in Mouse Retinal

Weeks after AAV9-hfCas13X.1-sg^{VEGFA} injection, mice were anesthetized and perfused with PBS and retina were isolated. Total RNA of retina were extracted and purified with Trizol (Ambion) and then transcribed into complementary DNA (HiScript Q RT SuperMix for qPCR, Vazyme, Biotech). Expression of hfCas13X.1, sg^{VEGFA} and VEGFA were detected with qPCR by Taqman probe (Bestar qPCR master mix, DBI-2041, DBI). mVEGFA qPCR primers are: Forward, 5′-GCTACTGCCGTCCGATTGAG-3′ (SEQ ID NO: 79); Reverse, 5′-CACTCCAGGGCTTCATCGTT-3′(SEQ ID NO: 80); probe, TCCAGGAGTACCCCGACGAGATAG (SEQ ID NO: 81), hfCas13X.1 qPCR primers are: Forward, 5′-CGGCGAGCAGGGTGATAAGA-3′ (SEQ ID NO: 82); Reverse, 5′-CCAGGTAGTGCAGTGCAAATT-3′ (SEQ ID NO: 83); probe, TCCTTGTGCCGCTTGGGATITGTG (SEQ ID NO: 84), sg^{VEGFA} qPCR primers are: Forward, 5′-GGTACTCCTGGAAGATGTCC-3′ (SEQ ID NO: 85); Reverse, 5′-TGTAATCACCCCACAAATCG-3′ (SEQ ID NO: 86); probe, ACCAGGGTCTGCTGGAGCAGCC (SEQ ID NO: 87).

### Laser-Induced CNV Mouse Model and CNV Staining

Three weeks after AAV9-hfCas13X.1-sg^{VEGFA} injection, mice were used for laser burn. Briefly, mice were anesthetized with mixture of zoletil (60 μg/g) and xylazine (10 μg/g) and pupils were dilated with dilating eye drops to enlarge the pupil size. Laser photocoagulation was performed using NOVUS Spectra (LUMENIS). The laser parameters used in this study were: 532 nm wave length, 70 ms exposure time, 240 mW power and 50 μm spot size. 4 laser burns around the optic disc were induced. Mice with vitreous hemorrhage were excluded in the study. CNV analysis was conducted 7 days later after laser burn. Mice were perfused with PBS followed by ice-cold 4% paraformaldehyde (PFA) and the eyes were then fixed with PFA for 2 hours. The retina was removed from the eyes, and only RPE/choroid/scleral complex was stained with isolectin-B4 (IB4, 10 μg/mL, 121413, Life Technologies) overnight. RPE complexes were flat-mounted and CNV images were obtained with Nikon microscope. The area of CNV was quantified using ImageJ software by a blind observer.

### Laser Induced CNV NHP Model

Both eyes of the monkeys were instilled with 1-2 drops of tropicamide eye drops to dilute the pupils. Thereafter, the animals were sedated with 10-30 mg/kg ketamine intramuscular injection, and anesthetized with xylazine intramuscular injection at a dosage of 0.5˜0.75 mg/kg. Oxybuprocaine Eye Drops were instilled to the conjunctival sac for surface anesthesia. Carbomer Eye Drops (0.2%) were smeared onto the back surface of laser lens to help observe the fundus clearly. Then laser photocoagulation was conducted in the perimacular region which were about 1.5-2 disk diameter from the foveal center, using a setting of 50 μm spot size, 0.1 second's duration and 400-700 mW intensity. The eyes of animals were smeared with ofloxacin eye ointment after laser photocoagulation and animals were put on the blanket to keep warm, and put back to the cages after they are conscious. Growth of CNV were measured with area and SHRM by FP FFA and OCT examination at different time points post laser. For FFA, animals were given Fluorescein Sodium Injection (10 mg/kg, 100 mg/mL) by quick intravenous injection before fluorescein angiography after fundus photography. Visual function was measured with ERG at week 9 post laser.

Exemplary Sequences
pAAV9-hfCas13X.1-sg^{VEGFA}

Condon optimized wild type Cas13e (SEQ ID NO: 1),
without the TAG stop codon
ATGGCCCAGGTGAGCAAGCAGACCTCCAAGAAGAGGGAGCTGAGCATCGACGAGTACCAGGGCGCCCG
GAAGTGGTGCTTCACCATTGCCTTCAACAAGGCCCTGGTGAACCGGGACAAGAACGACGGCCTGTTCG
TGGAAAGCCTGCTGAGACACGAGAAGTACAGCAAGCACGACTGGTACGACGAAGATACCCGGGCCCTG
ATCAAGTGCAGCACCCAGGCCGCCAACGCCAAGGCTGAAGCCCTGCGGAACTACTTCAGTCACTACCG
GCATAGCCCTGGCTGCCTGACCTTCACCGCCGAGGACGAACTGCGGACCATCATGGAGAGAGCCTATG
AGCGGGCCATCTTCGAGTGCAGAAGAAGAGAGACAGAGGTGATCATCGAGTTTCCCAGCCTGTTCGAG
GGCGACCGGATCACCACCGCCGGCGTGGTGTTTTTCGTGAGCTTTTTCGTGGAAAGAAGAGTGCTGGA
TCGGCTGTATGGAGCCGTGTCCGGCCTGAAGAAGAATGAGGGACAGTACAAGCTGACCCGGAAGGCCC
TGAGCATGTACTGCCTGAAGGACAGCAGATTCACCAAGGCCTGGGATAAGCGGGTGCTGCTGTTCAGA
GACATCCTGGCCCAGCTGGGAAGAATCCCCGCCGAGGCCTACGAGTACTACCACGGCGAGCAGGGTGA
TAAGAAGAGAGCTAACGACAATGAGGGCACAAATCCCAAGCGGCACAAGGACAAGTTCATCGAATTTG
CACTGCACTACCTGGAAGCCCAGCACAGCGAGATCTGCTTCGGCAGACGCCACATCGTGCGGGAAGAG
GCCGGCGCCGGCGATGAGCACAAGAAGCACCGGACCAAGGGAAAGGTGGTGGTGGACTTCAGCAAGAA
GGACGAGGACCAGAGCTACTATATCTCCAAGAACAACGTGATCGTGCGGATCGACAAGAACGCCGGCC
CTAGAAGCTACCGGATGGGCCTGAACGAGCTGAAGTACCTCGTGCTGCTGAGCCTGCAGGGGAAGGGC
GACGATGCCATCGCCAAGCTGTACAGATACAGACAGCACGTGGAGAACATCCTGGATGTGGTGAAGGT
GACCGATAAGGATAACCACGTGTTCCTGCCCCGCTTCGTGCTGGAGCAGCACGGCATCGGCAGAAAGG
CCTTCAAGCAGCGGATCGATGGACGGGTGAAGCACGTGCGGGGCGTGTGGGAGAAGAAGAAGGCCGCC
ACCAATGAAATGACCCTGCACGAGAAGGCCAGAGACATCCTGCAGTACGTGAACGAAAACTGCACCCG
GTCCTTCAACCCTGGCGAATACAACAGACTGCTGGTGTGCCTGGTGGGCAAGGACGTGGAGAACTTTC
AGGCCGGCCTGAAGCGGCTGCAGCTGGCCGAAAGGATCGATGGCCGGGTGTACTCCATCTTCGCCCAG
ACCAGCACCATCAATGAGATGCACCAGGTGGTGTGCGACCAGATCCTGAACCGGCTGTGCAGAATCGG
CGACCAGAAGCTGTACGATTACGTGGGACTGGGCAAGAAGGACGAAATCGACTACAAGCAGAAGGTGG
CCTGGTTCAAGGAGCACATCAGCATCCGGAGAGGATTCCTGAGAAAGAAGTTCTGGTACGATAGCAAG
AAGGGATTCGCAAAGCTGGTGGAGGAACACCTGGAGTCCGGCGGCGGCCAGCGCGACGTGGGCCTGGA
CAAGAAGTACTACCACATCGACGCCATCGGCAGATTCGAGGGCGCCAACCCCGCCCTGTACGAGACCC
TGGCCAGAGATCGGCTGTGCCTCATGATGGCCCAGTACTTCCTGGGCAGCGTGAGAAAGGAACTGGGC
AACAAGATTGTGTGGAGCAACGACAGCATCGAACTGCCTGTGGAAGGCTCTGTGGGAAATGAGAAGAG
CATCGTGTTCTCCGTGTCTGACTACGGCAAGCTGTACGTGCTGGACGATGCCGAATTCCTGGGCCGGA
TCTGCGAATACTTCATGCCCCACGAAAAGGGCAAGATCCGGTACCACACAGTGTACGAAAAGGGCTTT
AGAGCATACAACGACCTGCAGAAGAAGTGCGTGGAGGCCGTGCTGGCTTTCGAAGAGAAGGTGGTGAA
GGCCAAGAAGATGAGCGAGAAGGAAGGCGCCCACTACATCGACTTCCGGGAGATCCTGGCCCAGACCA
TGTGCAAGGAGGCCGAaAAGACCGCAGTGAACAAGGTGAGACGCGCCTTCTTCCACCACCACCTGAAG
TTCGTGATTGACGAGTTCGGCCTGTTCAGCGACGTGATGAAGAAGTACGGCATCGAGAAGGAATGGAA
GTTCCCTGTCAAG
Amino acid sequence wild type Cas13e (SEQ ID NO: 4)
MAQVSKQTSKKRELSIDEYQGARKWCFTIAFNKALVNRDKNDGLFVESLLRHEKYSKHDWYDEDTRAL
IKCSTQAANAKAEALRNYFSHYRHSPGCLTFTAEDELRTIMERAYERAIFECRRRETEVIIEFPSLFE
GDRITTAGVVFFVSFFVERRVLDRLYGAVSGLKKNEGQYKLTRKALSMYCLKDSRFTKAWDKRVLLFR
DILAQLGRIPAEAYEYYHGEQGDKKRANDNEGTNPKRHKDKFIEFALHYLEAQHSEICFGRRHIVREE
AGAGDEHKKHRTKGKVVVDFSKKDEDQSYYISKNNVIVRIDKNAGPRSYRMGLNELKYLVLLSLQGKG
DDAIAKLYRYRQHVENILDVVKVTDKDNHVFLPRFVLEQHGIGRKAFKQRIDGRVKHVRGVWEKKKAA
TNEMTLHEKARDILQYVNENCTRSFNPGEYNRLLVCLVGKDVENFQAGLKRLQLAERIDGRVYSIFAQ
TSTINEMHQVVCDQILNRLCRIGDQKLYDYVGLGKKDEIDYKQKVAWFKEHISIRRGFLRKKFWYDSK
KGFAKLVEEHLESGGGQRDVGLDKKYYHIDAIGRFEGANPALYETLARDRLCLMMAQYFLGSVRKELG
NKIVWSNDSIELPVEGSVGNEKSIVFSVSDYGKLYVLDDAEFLGRICEYFMPHEKGKIRYHTVYEKGF
RAYNDLQKKCVEAVLAFEEKVVKAKKMSEKEGAHYIDFREILAQTMCKEAEKTAVNKVRRAFFHHHLK
FVIDEFGLFSDVMKKYGIEKEWKFPVK
hfCas13X.1 (SEQ ID NO: 5)
ATGGCCCAGGTGAGCAAGCAGACCTCCAAGAAGAGGGAGCTGAGCATCGACGAGTACCAGGGCGCCCG
GAAGTGGTGCTTCACCATTGCCTTCAACAAGGCCCTGGTGAACCGGGACAAGAACGACGGCCTGTTCG
TGGAAAGCCTGCTGAGACACGAGAAGTACAGCAAGCACGACTGGTACGACGAAGATACCCGGGCCCTG
ATCAAGTGCAGCACCCAGGCCGCCAACGCCAAGGCTGAAGCCCTGCGGAACTACTTCAGTCACTACCG
GCATAGCCCTGGCTGCCTGACCTTCACCGCCGAGGACGAACTGCGGACCATCATGGAGAGAGCCTATG
AGCGGGCCATCTTCGAGTGCAGAAGAAGAGAGACAGAGGTGATCATCGAGTTTCCCAGCCTGTTCGAG
GGCGACCGGATCACCACCGCCGGCGTGGTGTTTTTCGTGAGCTTTTTCGTGGAAAGAAGAGTGCTGGA
TCGGCTGTATGGAGCCGTGTCCGGCCTGAAGAAGAATGAGGGACAGTACAAGCTGACCCGGAAGGCCC
TGAGCATGTACTGCCTGAAGGACAGCAGATTCACCAAGGCCTGGGATAAGCGGGTGCTGCTGTTCAGA
GACATCCTGGCCCAGCTGGGAAGAATCCCCGCCGAGGCCTACGAGTACTACCACGGCGAGCAGGGTGA
TAAGAAGAGAGCTAACGACAATGAGGGCACAAATCCCAAGCGGCACAAGGACAAGTTCATCGAATTTG
CACTGCACTACCTGGAAGCCCAGCACAGCGAGATCTGCTTCGGCAGACGCCACATCGTGCGGGAAGAG
GCCGGCGCCGGCGATGAGCACAAGAAGCACCGGACCAAGGGAAAGGTGGTGGTGGACTTCAGCAAGAA
GGACGAGGACCAGAGCTACTATATCTCCAAGAACAACGTGATCGTGCGGATCGACAAGAACGCCGGCC
CTAGAAGCTACCGGATGGGCCTGAACGAGCTGAAGTACCTCGTGCTGCTGAGCCTGCAGGGGAAGGGC
GACGATGCCATCGCCAAGCTGTACAGATACAGACAGCACGTGGAGAACATCCTGGATGTGGTGAAGGT
GACCGATAAGGATAACCACGTGTTCCTGCCCCGCTTCGTGCTGGAGCAGCACGGCATCGGCAGAAAGG
CCTTCAAGCAGCGGATCGATGGACGGGTGAAGCACGTGCGGGGCGTGTGGGAGAAGAAGAAGGCCGCC
ACCAATGAAATGACCCTGCACGAGAAGGCCAGAGACATCCTGCAGTACGTGAACGAAAACTGCACCCG
GTCCTTCAACCCTGGCGAATACAACAGACTGCTGGTGTGCCTGGTGGGCAAGGACGTGGAGAACTTTC
AGGCCGGCCTGAAGCGGCTGCAGCTGGCCGAAAGGATCGATGGCCGGGTGTACTCCATCTTCGCCCAG
ACCAGCACCATCAATGAGATGCACCAGGTGGTGTGCGACCAGATCCTGAACCGGCTGTGCAGAATCGG
CGACCAGAAGCTGTACGATTACGTGGGACTGGGCAAGAAGGACGAAATCGACTACAAGCAGAAGGTGG
CCTGGTTCAAGGAGCACATCAGCATCCGGAGAGGATTCCTGAGAAAGAAGTTCTGGTACGATAGCAAG
AAGGGATTCGCAAAGCTGGTGGAGGAACACCTGGAGTCCGGCGGCGGCCAGCGCGACGTGGGCCTGGA
CAAGAAGTACTACCACATCGACGCCATCGGCAGATTCGAGGGCGCCAACCCCGCCCTGTACGAGACCC
TGGCCAGAGATCGGCTGTGCCTCATGATGGCCCAGTACTTCCTGGGCAGCGTGAGAAAGGAACTGGGC
AACAAGATTGTGTGGAGCAACGACAGCATCGAACTGCCTGTGGAAGGCTCTGTGGGAAATGAGAAGAG
CATCGTGTTCTCCGTGTCTGACTACGGCAAGCTGTACGTGCTGGACGATGCCGAATTCCTGGGCCGGA
TCTGCGAATACTTCATGCCCCACGAAAAGGGCAAGATCCGGGCCCACACAGTGGCCGAAAAGGGCTTT
AGAGCATACAACGACCTGCAGAAGAAGTGCGTGGAGGCCGTGCTGGCTTTCGAAGAGAAGGTGGTGAA
GGCCAAGAAGATGAGCGAGAAGGAAGGCGCCCACTACATCGACTTCCGGGAGATCCTGGCCCAGACCA
TGTGCAAGGAGGCCGAAAAGACCGCAGTGAACAAGGTGAGACGCGCCTTCTTCCACCACCACCTGAAG
TTCGTGATTGACGAGTTCGGCCTGTTCAGCGACGTGATGAAGAAGTACGGCATCGAGAAGGAATGGAA
GTTCCCTGTCAAG
Amino acid sequence of Cas13X.1 (SEQ ID NO: 2)
MAQVSKQTSKKRELSIDEYQGARKWCFTIAFNKALVNRDKNDGLFVESLLRHEKYSKHDWYDEDTRAL
IKCSTQAANAKAEALRNYFSHYRHSPGCLTFTAEDELRTIMERAYERAIFECRRRETEVIIEFPSLFE
GDRITTAGVVFFVSFFVERRVLDRLYGAVSGLKKNEGQYKLTRKALSMYCLKDSRFTKAWDKRVLLFR
DILAQLGRIPAEAYEYYHGEQGDKKRANDNEGTNPKRHKDKFIEFALHYLEAQHSEICFGRRHIVREE
AGAGDEHKKHRTKGKVVVDFSKKDEDQSYYISKNNVIVRIDKNAGPRSYRMGLNELKYLVLLSLQGKG
DDAIAKLYRYRQHVENILDVVKVTDKDNHVFLPRFVLEQHGIGRKAFKQRIDGRVKHVRGVWEKKKAA
TNEMTLHEKARDILQYVNENCTRSFNPGEYNRLLVCLVGKDVENFQAGLKRLQLAERIDGRVYSIFAQ
TSTINEMHQVVCDQILNRLCRIGDQKLYDYVGLGKKDEIDYKQKVAWFKEHISIRRGFLRKKFWYDSK
KGFAKLVEEHLESGGGQRDVGLDKKYYHIDAIGRFEGANPALYETLARDRLCLMMAQYFLGSVRKELG
NKIVWSNDSIELPVEGSVGNEKSIVFSVSDYGKLYVLDDAEFLGRICEYFMPHEKGKIR(Xaa)HTV
(Xaa) EKGFRAYNDLQKKCVEAVLAFEEKVVKAKKMSEKEGAHYIDFREILAQTMCKEAEKTAVNKV
RRAFFHHHLKFV(Xaa)DEFGLFSDVMKKYGIEKEWKFPVK
In SEQ ID NO: 2, Xaa at residue 672 is defined as: any amino acid,
except Y when Xaa at 676 is Y and Xaa at 751 is 1.
Xaa at residue 676 is defined as: any amino acid, except Y when Xaa
at 672 is Y and Xaa at 751 is I.
Xaa at residue 751 is defined as: any amino acid, except I when Xaa
at 672 and Xaa at 676 are both Y.
Amino acid sequence of Cas13X.1 (SEQ ID NO: 3)
MAQVSKQTSKKRELSIDEYQGARKWCFTIAFNKALVNRDKNDGLFVESLLRHEKYSKHDWYDEDTRAL
IKCSTQAANAKAEALRNYFSHYRHSPGCLTFTAEDELRTIMERAYERAIFECRRRETEVIIEFPSLFE
GDRITTAGVVFFVSFFVERRVLDRLYGAVSGLKKNEGQYKLTRKALSMYCLKDSRFTKAWDKRVLLFR
DILAQLGRIPAEAYEYYHGEQGDKKRANDNEGTNPKRHKDKFIEFALHYLEAQHSEICFGRRHIVREE
AGAGDEHKKHRTKGKVVVDFSKKDEDQSYYISKNNVIVRIDKNAGPRSYRMGLNELKYLVLLSLQGKG
DDAIAKLYRYRQHVENILDVVKVTDKDNHVFLPRFVLEQHGIGRKAFKQRIDGRVKHVRGVWEKKKAA
TNEMTLHEKARDILQYVNENCTRSFNPGEYNRLLVCLVGKDVENFQAGLKRLQLAERIDGRVYSIFAQ
TSTINEMHQVVCDQILNRLCRIGDQKLYDYVGLGKKDEIDYKQKVAWFKEHISIRRGFLRKKFWYDSK
KGFAKLVEEHLESGGGQRDVGLDKKYYHIDAIGRFEGANPALYETLARDRLCLMMAQYFLGSVRKELG
NKIVWSNDSIELPVEGSVGNEKSIVFSVSDYGKLYVLDDAEFLGRICEYFMPHEKGKIRAHTVAEKGF
RAYNDLQKKCVEAVLAFEEKVVKAKKMSEKEGAHYIDFREILAQTMCKEAEKTAVNKVRRAFFHHHLK
FVIDEFGLFSDVMKKYGIEKEWKFPVK
DR SEQ ID NO: 6
GCTGGAGCAGCCCCCGATTTGTGGGGTGATTACAGC
Sg1 SEQ ID NO: 7
GTGCTGTAGGAAGCTCATCTCTCCTATGTG
Sg2 SEQ ID NO: 8
GGTACTCCTGGAAGATGTCCACCAGGGTCT
5’ ITR (SEQ ID NO: 10)
CCTGCAGGCAGCTGCGCGCTCGCTCGCTCACTGAGGCCGCCCGGGCAAAGCCCGGGCGTCGGGCGACC
TTTGGTCGCCCGGCCTCAGTGAGCGAGCGAGCGCGCAGAGAGGGAGTGGCCAACTCCATCACTAGGGG
TTCCT
3’ ITR (SEQ ID NO: 11)
AGGAACCCCTAGTGATGGAGTTGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGAGCGCGCAGCTGCCT
GCAGG
EFS promoter (SEQ ID NO: 12)
TAGGTCTTGAAAGGAGTGGGAATTGGCTCCGGTGCCCGTCAGTGGGCAGAGCGCACATCGCCCACAGT
CCCCGAGAAGTTGGGGGGAGGGGTCGGCAATTGATCCGGTGCCTAGAGAAGGTGGCGCGGGGTAAACT
GGGAAAGTGATGTCGTGTACTGGCTCCGCCTTTTTCCCGAGGGTGGGGGAGAACCGTATATAAGTGCA
GTAGTCGCCGTGAACGTTCTTTTTCGCAACGGGTTTGCCGCCAGAACACAGGGTTGG
Kozak (SEQ ID NO: 13)
GCCACCATG
SV40 NLS (SEQ ID NO: 14)
CCCAAGAAGAAGCGGAAGGTG
SV40 poly A (SEQ ID NO: 15)
AACTTGTTTATTGCAGCTTATAATGGTTACAAATAAAGCAATAGCATCACAAATTTCACAAATAAAGC
ATTTTTTTCACTGCATTCTAGTTGTGGTTTGTCCAAACTCATCAATGTATCTTATCATGTCTGGATC
U6 promoter (SEQ ID NO: 16)
GAGGGCCTATTTCCCATGATTCCTTCATATTTGCATATACGATACAAGGCTGTTAGAGAGATAATTGG
AATTAATTTGACTGTAAACACAAAGATATTAGTACAAAATACGTGACGTAGAAAGTAATAATTTCTTG
GGTAGTTTGCAGTTTTAAAATTATGTTTTAAAATGGACTATCATATGCTTACCGTAACTTGAAAGTAT
TTCGATTTCTTGGCTTTATATATCTTGTGGAAAGGAC
pAAV9-hfCas13X.1-sg^{VEGFA} ITR-to-ITR (SEQ ID NO: 17)
CCTGCAGGCAGCTGCGCGCTCGCTCGCTCACTGAGGCCGCCCGGGCAAAGCCCGGGCGTCGGGCGACC
TTTGGTCGCCCGGCCTCAGTGAGCGAGCGAGCGCGCAGAGAGGGAGTGGCCAACTCCATCACTAGGGG
TTCCTGCGGCCATCGATGGCGCGCCGGATTCGACATTGATTATTGATAGGTCTTGAAAGGAGTGGGAA
TTGGCTCCGGTGCCCGTCAGTGGGCAGAGCGCACATCGCCCACAGTCCCCGAGAAGTTGGGGGGAGGG
GTCGGCAATTGATCCGGTGCCTAGAGAAGGTGGCGCGGGGTAAACTGGGAAAGTGATGTCGTGTACTG
GCTCCGCCTTTTTCCCGAGGGTGGGGGAGAACCGTATATAAGTGCAGTAGTCGCCGTGAACGTTCTTT
TTCGCAACGGGTTTGCCGCCAGAACACAGGGTTGGACCGGTGCCACCATGCCCAAGAAGAAGCGGAAG
GTGGCCCAGGTGAGCAAGCAGACCTCCAAGAAGAGGGAGCTGAGCATCGACGAGTACCAGGGCGCCCG
GAAGTGGTGCTTCACCATTGCCTTCAACAAGGCCCTGGTGAACCGGGACAAGAACGACGGCCTGTTCG
TGGAAAGCCTGCTGAGACACGAGAAGTACAGCAAGCACGACTGGTACGACGAAGATACCCGGGCCCTG
ATCAAGTGCAGCACCCAGGCCGCCAACGCCAAGGCTGAAGCCCTGCGGAACTACTTCAGTCACTACCG
GCATAGCCCTGGCTGCCTGACCTTCACCGCCGAGGACGAACTGCGGACCATCATGGAGAGAGCCTATG
AGCGGGCCATCTTCGAGTGCAGAAGAAGAGAGACAGAGGTGATCATCGAGTTTCCCAGCCTGTTCGAG
GGCGACCGGATCACCACCGCCGGCGTGGTGTTTTTCGTGAGCTTTTTCGTGGAAAGAAGAGTGCTGGA
TCGGCTGTATGGAGCCGTGTCCGGCCTGAAGAAGAATGAGGGACAGTACAAGCTGACCCGGAAGGCCC
TGAGCATGTACTGCCTGAAGGACAGCAGATTCACCAAGGCCTGGGATAAGCGGGTGCTGCTGTTCAGA
GACATCCTGGCCCAGCTGGGAAGAATCCCCGCCGAGGCCTACGAGTACTACCACGGCGAGCAGGGTGA
TAAGAAGAGAGCTAACGACAATGAGGGCACAAATCCCAAGCGGCACAAGGACAAGTTCATCGAATTTG
CACTGCACTACCTGGAAGCCCAGCACAGCGAGATCTGCTTCGGCAGACGCCACATCGTGCGGGAAGAG
GCCGGCGCCGGCGATGAGCACAAGAAGCACCGGACCAAGGGAAAGGTGGTGGTGGACTTCAGCAAGAA
GGACGAGGACCAGAGCTACTATATCTCCAAGAACAACGTGATCGTGCGGATCGACAAGAACGCCGGCC
CTAGAAGCTACCGGATGGGCCTGAACGAGCTGAAGTACCTCGTGCTGCTGAGCCTGCAGGGGAAGGGC
GACGATGCCATCGCCAAGCTGTACAGATACAGACAGCACGTGGAGAACATCCTGGATGTGGTGAAGGT
GACCGATAAGGATAACCACGTGTTCCTGCCCCGCTTCGTGCTGGAGCAGCACGGCATCGGCAGAAAGG
CCTTCAAGCAGCGGATCGATGGACGGGTGAAGCACGTGCGGGGCGTGTGGGAGAAGAAGAAGGCCGCC
ACCAATGAAATGACCCTGCACGAGAAGGCCAGAGACATCCTGCAGTACGTGAACGAAAACTGCACCCG
GTCCTTCAACCCTGGCGAATACAACAGACTGCTGGTGTGCCTGGTGGGCAAGGACGTGGAGAACTTTC
AGGCCGGCCTGAAGCGGCTGCAGCTGGCCGAAAGGATCGATGGCCGGGTGTACTCCATCTTCGCCCAG
ACCAGCACCATCAATGAGATGCACCAGGTGGTGTGCGACCAGATCCTGAACCGGCTGTGCAGAATCGG
CGACCAGAAGCTGTACGATTACGTGGGACTGGGCAAGAAGGACGAAATCGACTACAAGCAGAAGGTGG
CCTGGTTCAAGGAGCACATCAGCATCCGGAGAGGATTCCTGAGAAAGAAGTTCTGGTACGATAGCAAG
AAGGGATTCGCAAAGCTGGTGGAGGAACACCTGGAGTCCGGCGGCGGCCAGCGCGACGTGGGCCTGGA
CAAGAAGTACTACCACATCGACGCCATCGGCAGATTCGAGGGCGCCAACCCCGCCCTGTACGAGACCC
TGGCCAGAGATCGGCTGTGCCTCATGATGGCCCAGTACTTCCTGGGCAGCGTGAGAAAGGAACTGGGC
AACAAGATTGTGTGGAGCAACGACAGCATCGAACTGCCTGTGGAAGGCTCTGTGGGAAATGAGAAGAG
CATCGTGTTCTCCGTGTCTGACTACGGCAAGCTGTACGTGCTGGACGATGCCGAATTCCTGGGCCGGA
TCTGCGAATACTTCATGCCCCACGAAAAGGGCAAGATCCGGGCCCACACAGTGGCCGAAAAGGGCTTT
AGAGCATACAACGACCTGCAGAAGAAGTGCGTGGAGGCCGTGCTGGCTTTCGAAGAGAAGGTGGTGAA
GGCCAAGAAGATGAGCGAGAAGGAAGGCGCCCACTACATCGACTTCCGGGAGATCCTGGCCCAGACCA
TGTGCAAGGAGGCCGAAAAGACCGCAGTGAACAAGGTGAGACGCGCCTTCTTCCACCACCACCTGAAG
TTCGTGATTGACGAGTTCGGCCTGTTCAGCGACGTGATGAAGAAGTACGGCATCGAGAAGGAATGGAA
GTTCCCTGTCAAGCCCAAGAAGAAGCGGAAGGTGTAGAAGCTTAACTTGTTTATTGCAGCTTATAATG
GTTACAAATAAAGCAATAGCATCACAAATTTCACAAATAAAGCATTTTTTTCACTGCATTCTAGTTGT
GGTTTGTCCAAACTCATCAATGTATCTTATCATGTCTGGATCACTAGTGAGGGCCTATTTCCCATGAT
TCCTTCATATTTGCATATACGATACAAGGCTGTTAGAGAGATAATTGGAATTAATTTGACTGTAAACA
CAAAGATATTAGTACAAAATACGTGACGTAGAAAGTAATAATTTCTTGGGTAGTTTGCAGTTTTAAAA
TTATGTTTTAAAATGGACTATCATATGCTTACCGTAACTTGAAAGTATTTCGATTTCTTGGCTTTATA
TATCTTGTGGAAAGGACGAAACACCGGCTGGAGCAGCCCCCGATTTGTGGGGTGATTACAGCGTGCTG
TAGGAAGCTCATCTCTCCTATGTGGCTGGAGCAGCCCCCGATTTGTGGGGTGATTACAGCGGTACTCC
TGGAAGATGTCCACCAGGGTCTGCTGGAGCAGCCCCCGATTTGTGGGGTGATTACAGCTTTTTTTACG
CGTTAGTTATTAATAGTGCGTCGGCCGCAGGAACCCCTAGTGATGGAGTTGGCCACTCCCTCTCTGCG
CGCTCGCTCGCTCACTGAGGCCGGGCGACCAAAGGTCGCCCGACGCCCGGGCTTTGCCCGGGCGGCCT
CAGTGAGCGAGCGAGCGCGCAGCTGCCTGCAGG
p-U6-shRNA-CMV-mCherry
Full length (SEQ ID NO: 49)
GAATTCACTGGCCGTCGTTTTACAACGTCGTGACTGGGAAAACCCTGGCGTTACCCAACTTAATCGCC
TTGCAGCACATCCCCCTTTCGCCAGCTGGCGTAATAGCGAAGAGGCCCGCACCGATGGATCCCGCCGT
TACATAACTTACGGTAAATGGCCCGCCTGGCTGACCGCCCAACGACCCCCGCCCATTGACGTCAATAA
TGACGTATGTTCCCATAGTAACGCCAATAGGGACTTTCCATTGACGTCAATGGGTGGAGTATTTACGG
TAAACTGCCCACTTGGCAGTACATCAAGTGTATCATATGCCAAGTACGCCCCCTATTGACGTCAATGA
CGGTAAATGGCCCGCCTGGCATTATGCCCAGTACATGACCTTATGGGACTTTCCTACTTGGCAGTACA
TCTACGTATTAGTCATCGCTATTACCATGGTGATGCGGTTTTGGCAGTACATCAATGGGCGTGGATAG
CGGTTTGACTCACGGGGATTTCCAAGTCTCCACCCCATTGACGTCAATGGGAGTTTGTTTTGGCACCA
AAATCAACGGGACTTTCCAAAATGTCGTAACAACTCCGCCCCATTGACGCAAATGGGCGGTAGGCGTG
TACGGTGGGAGGTCTATATAAGCAGAGCTGGTTTAGTGAACCGTCAGATCCGATGGTGAGCAAGGGCG
AGGAGGATAACATGGCCATCATCAAGGAGTTCATGCGCTTCAAGGTGCACATGGAGGGCTCCGTGAAC
GGCCACGAGTTCGAGATCGAGGGCGAGGGCGAGGGCCGCCCCTACGAGGGCACCCAGACCGCCAAGCT
GAAGGTGACCAAGGGTGGCCCCCTGCCCTTCGCCTGGGACATCCTGTCCCCTCAGTTCATGTACGGCT
CCAAGGCCTACGTGAAGCACCCCGCCGACATCCCCGACTACTTGAAGCTGTCCTTCCCCGAGGGCTTC
AAGTGGGAGCGCGTGATGAACTTCGAGGACGGCGGCGTGGTGACCGTGACCCAGGACTCCTCCCTGCA
GGACGGCGAGTTCATCTACAAGGTGAAGCTGCGCGGCACCAACTTCCCCTCCGACGGCCCCGTAATGC
AGAAGAAAACCATGGGCTGGGAGGCCTCCTCCGAGCGGATGTACCCCGAGGACGGCGCCCTGAAGGGC
GAGATCAAGCAGAGGCTGAAGCTGAAGGACGGCGGCCACTACGACGCTGAGGTCAAGACCACCTACAA
GGCCAAGAAGCCCGTGCAGCTGCCCGGCGCCTACAACGTCAACATCAAGTTGGACATCACCTCCCACA
ACGAGGACTACACCATCGTGGAACAGTACGAACGCGCCGAGGGCCGCCACTCCACCGGCGGCATGGAC
GAGCTGTACAAGTAACTCTTGGCCCGGATCCCTGTGCCTTCTAGTTGCCAGCCATCTGTTGTTTGCCC
CTCCCCCGTGCCTTCCTTGACCCTGGAAGGTGCCACTCCCACTGTCCTTTCCTAATAAAATGAGGAAA
TTGCATCGCATTGTCTGAGTAGGTGTCATTCTATTCTGGGGGGTGGGGTGGGGCAGGACAGCAAGGGG
GAGGATTGGGAAAACAATAGCAGGCATGCTGGGGATGCGGTGGGCTCTATGGAAGCTAGCAGCAAATC
CTTCCAGACCCAACCAAACCAATCGTAGTAACCATTCAGGAACGCAACCGCAGCTTAGACCAAAACAG
GAAGCTATGGGCCTGCTTAGGTGACGTCTCTCGTCAGGTTGAATGGCATGGTCGCTGGCTGGATGCAG
AAAGCTGGAAGTGTGTGTTTACCGCAGCATTAAAGCAGCAGGATGTTGTTCCTAACCTTGCCGGGAAT
GGCTTTGTGGTAATAGGCCAGTCAACCAGCAGGATGCGTGTAGGCGAATTTGCGGAGCTATTAGAGCT
TATACAGGCATTCGGTACAGAGCGTGGCGTTAAGTGGTCAGACGAAGCGAGACTGGCTCTGGAGTGGA
AAGCGAGATGGGGAGACAGGGCTGCGCCATAAATGTCGTTAGTTTCTCCGGTGGCAGGACGTCAGCAT
ATTTGCTCTGGCTAATGGAGCAAAAGCGACGGGCAGGTAAAGACGTGCATTACGTTTTCATGGATACA
GGTTGTGAACATCCAATGACATATCGGTTTGTCAGGGAAGTTGTGAAGTTCTGGGATATACCGCTCAC
CGTATTGCAGGTTGATATCAACCCGGAGCTTGGACAGCCAAATGGTTATACGGTATGGGAACCAAAGG
ATATTCAGACGCGAATGCCTGTTCTGAAGCCATTTATCGATATGGTAAAGAAATATGGCACTCCATAC
GTCGGCGGCGCGTTCTGCACTGACAGATTAAAACTCGTTCCCTTCACCAAATACTGTGATGACCATTT
CGGGCGAGGGAATTACACCACGTGGATTGGCATCAGAGCTGATGAACCGAAGCGGCTAAAGCCAAAGC
CTGGAATCAGATATCTTGCTGAACTGTCAGACTTTGAGAAGGAAGATATCCTCGCATGGTGGAAGCAA
CAACCATTCGATTTGCAAATACCGGAACATCTCGGTAACTGCATATTCTGCATTAAAAAATCAACGCA
AAAAATCGGACTTGCCTGCAAAGATGAGGAGGGATTGCAGCGTGTTTTTAATGAGGTCATCACGGGAT
CCCATGTGCGTGACGGACATCGGGAAACGCCAAAGGAGATTATGTACCGAGGAAGAATGTCGCTGGAC
GGTATCGCGAAAATGTATTCAGAAAATGATTATCAAGCCCTGTATCAGGACATGGTACGAGCTAAAAG
ATTCGATACCGGCTCTTGTTCTGAGTCATGCGAAATATTTGGAGGGCAGCTTGATTTCGACTTCGGGA
GGGAAGCTGCGCCATGCGATGTTATCGGTGCGGTGAATGCAAAGAAGATAACCGCTTCCGACCAAATC
AACCTTACTGGAATCGATGGTGTCTCCGGTGTGAAAGAACACCAACAGGGGTGTTACCACTACCGCAG
GAAAAGGAGGACGTGCCGCGAGACAGCGACGAAGTATCACCGACATAATCTGCGAAAACTGCAAATAC
CTTCCAACGAAACGCACCAGAAATAAACCCAAGCCAATCCCAAAAGAATCTGACGTAAAAACCTTCAA
CTACACGGCTCACCTGTGGGATATCCGGTGGCTAAGACGTCGTGCGAGGAAAACAAGGCCATTGACCA
AAATCGAAGTTACGAACAAGAAAGCGTCGAGCGAGCTTTAACGTGCGCTAACTGCGGTCAGAAGCTGC
ATGTGCTGGAAGTTCACGTGTGTGAGCACTGCTGCGCAGAACTGATGAGCGATCCGAATAGCTCGATG
CACGAGGAAGAAGGCCGCCGCTAAACCAGCGCGAAGACGATGTAAAAACGATGAATGCCGGGAATGGT
TTCACCCTGCATTCGCTAATCAGTGGTGGTGCTCTCCAGAGTGTGGAACCAAGATAGCACTCGAACGA
CGAAGTAAAGAACGCGAAAAAGCGGAAAAAGCAGCAGAGAAGAAACGACGACGAGAGGAGCAGAAACA
GAAAGATAAACTTAAGATTCGAAAACTCGCCTTAAAGCCCCGCAGTTACTGGATTAAACAAGCCCAAC
AAGCCGTAAACGCCTTCATCAGAGAAAGAGACCGCGACTTACCATGTATCTCGTGCGGAACGCTCACG
TCTGCTCAGTGGGATGCCGGACATTACCGGACAACTGCTGCGGCACCTCAACTCCGATTTAATGAACG
CAATATTCACAAGCAATGCGTGGTGTGCAACCAGCACAAAAGCGGAAATCTCGTTCCGTATCGCGTCG
AACTGATTAGCCGCATCGGGCAGGAAGCAGTAGACGAAATCGAATCAAACCATAACCGCCATCGCTGG
ACTATCGAAGAGTGCAAGGCGATCAAGGCAGAGTACCAACAGAAACTCAAAGACCTGCGAAATAGCAG
AAGTGAGGCCGCGCCACGTTCTCAGTAAAAACCATTCCAGACATGCTCGTTGAAGCATACGGAAATCA
GACAGAAGTAGCACGCAGACTGAAATGTAGTCGCGGTACGGTCAGAAAATACGTTGATGATAAAGACG
GGAAAATGCACGCCATCGTCAACGACGTTCTCATGGTTCATCGCGGATGGAGTGAAAGAGGCCCGCTA
TTACGAAAAAATTGATGGCAGCAAATACCGAAATATTTGGGTAGTTGGCGATCTGCACGGATGCTACA
CGAACCTGATGAACAAACTGGATACGATTGGATTCGACAACAAAAAAGACCTGCTTATCTCGGTGGGC
GATTTGGTTGATCGTGGTGCAGAGAACGTTGAATGCCTGGAATTAATCACATTCCCCTGGTTCAGAGC
TGTACGTGGAAACCATGAGCAAATGATGATTGATGGCTTATCAGAGCGTGGAAACGTTAATCACTGGC
TGCTTAATGGCGGTGGCTGGTTCTTTAATCTCGATTACGACAAAGAAATTCTGGCTAAAGCTCTTGCC
CATAAAGCAGATGAACTTCCGTTAATCATCGAACTGGTGAGCAAAGATAAAAAATATGTTATCTGCCA
CGCCGATTATCCCTTTGACGAATACGAGTTTGGAAAGCCAGTTGATCATCAGCAGGTAATCTGGAACC
GCGAACGAATCAGCAACTCACAAAACGGGATCGTGAAAGAAATCAAAGGCGCGGACACGTTCATCTTT
GGTCATACGCCAGCAGTGAAACCACTCAAGTTTGCCAACCAAATGTATATCGATACCGGCGCAGTGTT
CTGCGGAAACCTAACATTGATTCAGGTACAGGGAGAAGGCGCGCCAGACTCGAAAGCGTAGCTACGCG
GAACCCCTATTTGTTTATTTTTCTAAATACATTCAAATATGTATCCGCTCATGAGACAATAACCCTGA
TAAATGCTTCAATAATATTGAAAAAGGAAGAGTATGAGTATTCAACATTTCCGTGTCGCCCTTATTCC
CTTTTTTGCGGCATTTTGCCTTCCTGTTTTTGCTCACCCAGAAACGCTGGTGAAAGTAAAAGATGCTG
AAGATCAGTTGGGTGCACGAGTGGGTTACATCGAACTGGATCTCAACAGCGGTAAGATCCTTGAGAGT
TTTCGCCCCGAAGAACGTTTTCCAATGATGAGCACTTTTAAAGTTCTGCTATGTGGCGCGGTATTATC
CCGTATTGACGCCGGGCAAGAGCAACTCGGTCGCCGCATACACTATTCTCAGAATGACTTGGTTGAGT
ACTCACCAGTCACAGAAAAGCATCTTACGGATGGCATGACAGTAAGAGAATTATGCAGTGCTGCCATA
ACCATGAGTGATAACACTGCGGCCAACTTACTTCTGACAACGATCGGAGGACCGAAGGAGCTAACCGC
TTTTTTGCACAACATGGGGGATCATGTAACTCGCCTTGATCGTTGGGAACCGGAGCTGAATGAAGCCA
TACCAAACGACGAGCGTGACACCACGATGCCTGTAGCAATGGCAACAACGTTGCGCAAACTATTAACT
GGCGAACTACTTACTCTAGCTTCCCGGCAACAATTAATAGACTGGATGGAGGCGGATAAAGTTGCAGG
ACCACTTCTGCGCTCGGCCCTTCCGGCTGGCTGGTTTATTGCTGATAAATCTGGAGCCGGTGAGCGTG
GGTCTCGCGGTATCATTGCAGCACTGGGGCCAGATGGTAAGCCCTCCCGTATCGTAGTTATCTACACG
ACGGGGAGTCAGGCAACTATGGATGAACGAAATAGACAGATCGCTGAGATAGGTGCCTCACTGATTAA
GCATTGGTAACTGTCAGACCAAGTTTACTCATATATACTTTAGATTGATTTAAAACTTCATTTTTAAT
TTAAAAGGATCTAGGTGAAGATCCTTTTTGATAATCTCATGACCAAAATCCCTTAACGTGAGTTTTCG
TTCCACTGAGCGTCAGACCCCGTAGAAAAGATCAAAGGATCTTCTTGAGATCCTTTTTTTCTGCGCGT
AATCTGCTGCTTGCAAACAAAAAAACCACCGCTACCAGCGGTGGTTTGTTTGCCGGATCAAGAGCTAC
CAACTCTTTTTCCGAAGGTAACTGGCTTCAGCAGAGCGCAGATACCAAATACTGTTCTTCTAGTGTAG
CCGTAGTTAGGCCACCACTTCAAGAACTCTGTAGCACCGCCTACATACCTCGCTCTGCTAATCCTGTT
ACCAGTGGCTGCTGCCAGTGGCGATAAGTCGTGTCTTACCGGGTTGGACTCAAGACGATAGTTACCGG
ATAAGGCGCAGCGGTCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAAGGGAGAAAGGCGGA
CAGGTATCCGGTAAGCGGCAGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTTCCTGGCCTTTTGCTGGCC
TTTTGCTCACATGTTCTTTCCTGCGTTATCCCCTGATTCTCTCGAGATCTAGGTTTACGACAAG
CMV promoter (SEQ ID NO: 50)
CGTTACATAACTTACGGTAAATGGCCCGCCTGGCTGACCGCCCAACGACCCCCGCCCATTGACGTCAA
TAATGACGTATGTTCCCATAGTAACGCCAATAGGGACTTTCCATTGACGTCAATGGGTGGAGTATTTA
CGGTAAACTGCCCACTTGGCAGTACATCAAGTGTATCATATGCCAAGTACGCCCCCTATTGACGTCAA
TGACGGTAAATGGCCCGCCTGGCATTATGCCCAGTACATGACCTTATGGGACTTTCCTACTTGGCAGT
ACATCTACGTATTAGTCATCGCTATTACCATGGTGATGCGGTTTTGGCAGTACATCAATGGGCGTGGA
TAGCGGTTTGACTCACGGGGATTTCCAAGTCTCCACCCCATTGACGTCAATGGGAGTTTGTTTTGGCA
CCAAAATCAACGGGACTTTCCAAAATGTCGTAACAACTCCGCCCCATTGACGCAAATGGGCGGTAGGC
GTGTACGGTGGGAGGTCTATATAAGCAGAGCT
mCherry (SEQ ID NO: 51)
ATGGTGAGCAAGGGCGAGGAGGATAACATGGCCATCATCAAGGAGTTCATGCGCTTCAAGGTGCACAT
GGAGGGCTCCGTGAACGGCCACGAGTTCGAGATCGAGGGCGAGGGCGAGGGCCGCCCCTACGAGGGCA
CCCAGACCGCCAAGCTGAAGGTGACCAAGGGTGGCCCCCTGCCCTTCGCCTGGGACATCCTGTCCCCT
CAGTTCATGTACGGCTCCAAGGCCTACGTGAAGCACCCCGCCGACATCCCCGACTACTTGAAGCTGTC
CTTCCCCGAGGGCTTCAAGTGGGAGCGCGTGATGAACTTCGAGGACGGCGGCGTGGTGACCGTGACCC
AGGACTCCTCCCTGCAGGACGGCGAGTTCATCTACAAGGTGAAGCTGCGCGGCACCAACTTCCCCTCC
GACGGCCCCGTAATGCAGAAGAAAACCATGGGCTGGGAGGCCTCCTCCGAGCGGATGTACCCCGAGGA
CGGCGCCCTGAAGGGCGAGATCAAGCAGAGGCTGAAGCTGAAGGACGGCGGCCACTACGACGCTGAGG
TCAAGACCACCTACAAGGCCAAGAAGCCCGTGCAGCTGCCCGGCGCCTACAACGTCAACATCAAGTTG
GACATCACCTCCCACAACGAGGACTACACCATCGTGGAACAGTACGAACGCGCCGAGGGCCGCCACTC
CACCGGCGGCATGGACGAGCTGTACAAGTAA
bGH polyA (SEQ ID NO: 52)
CTGTGCCTTCTAGTTGCCAGCCATCTGTTGTTTGCCCCTCCCCCGTGCCTTCCTTGACCCTGGAAGGT
GCCACTCCCACTGTCCTTTCCTAATAAAATGAGGAAATTGCATCGCATTGTCTGAGTAGGTGTCATTC
TATTCTGGGGGGTGGGGTGGGGCAGGACAGCAAGGGGGAGGATTGGGAAAACAATAGCAGGCATGCTG
GGGATGCGGTGGGCTCTATGG

1. A recombinant adeno-associate virus (rAAV) vector genome, comprising:
(1) a Cas13X polynucleotide encoding a Cas13X polypeptide, said Cas13X polynucleotide being at least 80%, 85%, 90%, 95%, 96%, 97%, 98%, 99%, 99.2%, 99.4%, 99.6%, 99.8%, or 99.9% identical to SEQ ID NO: 1, said Cas13X polypeptide comprising 1-3 substitutions at Y672, Y676, and/or I751 of SEQ ID NO: 4, and having substantially the same (e.g., at least about 80%, 90%, 95%, 99% or more) guide RNA-specific nuclease activity as SEQ ID NO: 4 and substantially no (e.g., at most 20%, 15%, 10%, 5%) collateral (guide RNA-independent) nuclease activity of SEQ ID NO: 4; and,
(2) a polyA signal sequence 3′ to the Cas13X polynucleotide;
optionally, said Cas13X polypeptide has the amino acid sequence of SEQ ID NO: 2 or 3.

2-3. (canceled)

4. The rAAV vector genome of claim 1, further comprising a promoter operably linked to and drives the transcription from the Cas13X polynucleotide, wherein the promoter is optionally:
1) a ubiquitous promoter,
2) a tissue-specific promoter;
3) a constitutive promoter; or
4) an inducible promoter.

5-8. (canceled)

9. The rAAV vector genome of claim 4, wherein the promoter is selected from the group consisting of a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter, optionally wherein the promoter is the elongation factor 1e short (EFS) promoter, such as SEQ ID NO: 12.

10. (canceled)

11. The rAAV vector genome of claim 1, further comprising a coding sequence for a nuclear localization sequence (NLS) fused N-terminal, C-terminal, or internally to the Cas13X polypeptide, and/or a coding sequence for a nuclear export signal (NES) fused N-terminal, C-terminal, or internally to the Cas13X polypeptide.

12-17. (canceled)

18. The rAAV vector genome of claim 1, further comprising a second transcription unit comprising an RNA pol III promoter, wherein said second transcription unit is 3′ to the Cas13X polynucleotide.

19. (canceled)

20. The rAAV vector genome of claim 18, wherein said second transcription unit further comprises a second coding sequence operably linked to the RNA pol III promoter, encoding one or more single guide RNAs (sgRNAs) each complementary to a target RNA sequence, and each capable of directing said Cas13X polypeptide to cleave said target RNA; optionally, each said sgRNA comprises a direct repeat (DR) sequence that binds the Cas13X polypeptide, wherein the DR sequence is optionally a nucleic sequence having at least 90% identity to SEQ ID NO: 6, at most 1, 2, 3, 4, or 5 nucleotide differences from SEQ ID NO: 6, and/or substantially the same secondary structure as that of SEQ ID NO: 6.

21-22. (canceled)

23. The rAAV vector genome of claim 20, wherein the target RNA is a transcript (e.g., mRNA) of a target gene associated with an eye disease or disorder, wherein the eye disease or disorder is optionally amoebic keratitis, fungal keratitis, bacterial keratitis, viral keratitis, onchorcercal keratitis, keratoconjunctivitis, bacterial keratoconjunctivitis, viral keratoconjunctivitis, vernal keratoconjunctivitis, atopic keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, noninfectious uveitis, anterior uveitis, posterior uveitis (including toxoplasmosis), pan-uveitis, an inflammatory disease of the vitreous or retina, endophthalmitis prophylaxis and treatment, macular edema, macular degeneration, wet age related macular degeneration (wet AMD), dry age related macular degeneration (dry AMD), diabetic macular edema (DME), allergic conjunctivitis, proliferative and non-proliferative diabetic retinopathy, hypertensive retinopathy, an autoimmune disease of the retina, primary and metastatic intraocular melanoma, other intraocular metastatic tumors, open angle glaucoma, Stargardt's disease, Fundus Flavimaculatus, closed angle glaucoma, pigmentary glaucoma, retinitis pigmentosa (RP), Leber's congenital amaurosis (LCA), Usher's syndrome, choroideremia, a rod-cone or cone-rod dystrophy, a ciliopathy, a mitochondrial disorder, progressive retinal atrophy, a degenerative retinal disease, geographic atrophy, a familial or acquired maculopathy, a retinal photoreceptor disease, a retinal pigment epithelial-based disease, cystoid macular edema, retinal detachment, traumatic retinal injury, iatrogenic retinal injury, macular holes, macular telangiectasia, a ganglion cell disease, an optic nerve cell disease, optic neuropathy, ischemic retinal disease, retinopathy of prematurity, retinal vascular occlusion, familial macroaneurysm, a retinal vascular disease, an ocular vascular diseases, a vascular disease, an ischemic optic neuropathy disease, diabetic retinal oedema, senile macular degeneration due to sub-retinal neovascularization, myopic retinopathy, retinal ischemia, choroidal vascular insufficiency, choroidal thrombosis and neovascular retinopathies resulting from carotoid artery ischemia, corneal neovascularisation, a corneal disease or opacification with an exudative or inflammatory component, diffuse lamellar keratitis, neovascularisation due to penetration of the eye or contusive ocular injury, rubosis iritis, Fuchs' heterochromic iridocyclitis, chronic uveitis, anterior uveitis, inflammatory conditions resulting from surgeries such as LASIK, LASEK, refractive surgery, IOL implantation: irreversible corneal oedema as a complication of cataract surgery, oedema as a result of insult or trauma, inflammation, infectious and non-infectious conjunctivitis, iridocyclitis, iritis, scleritis, episcleritis, superficial punctuate keratitis, keratoconus, posterior polymorphous dystrophy, Fuch's dystrophies, aphakic and pseudophakic bullous keratopathy, corneal oedema, scleral disease, ocular cicatrcial pemphigoid, pars planitis, Posner Schlossman syndrome, Behcet's disease, Vogt-Koyanagi-Harada syndrome, hypersensitivity reactions, ocular surface disorders, conjunctival oedema, Toxoplasmosis chorioretinitis, inflammatory pseudotumor of the orbit, chemosis, conjunctival venous congestion, periorbiatal cellulits, acute dacroycystitis, non-specific vasculitis, sarcoidosis, cytomegalovirus infection, and combinations thereof, optionally wherein the eye disease or disorder is wet age related macular degeneration (wet AMD).

24-25. (canceled)

26. The rAAV vector genome of claim 23, wherein the target gene is selected from the group consisting of Vascular Endothelial Growth Factor A (VEGFA), complement factor H (CFH), age-related maculopathy susceptibility 2 (ARMS2), HtrA serine peptidase 1 (HTRA1), ATP Binding Cassette Subfamily A Member 4 (ABCA4), Peripherin-2 (PRPH2), fibulin-5 (FBLN5), ERCC Excision Repair 6 Chromatin Remodeling Factor (ERCC6), Retina And Anterior Neural Fold Homeobox 2 (RAX2), Complement C3 (C3), Toll Like Receptor 4 (TLR4), Cystatin C (CST3), CX3C Chemokine Receptor 1 (CX3CR1), complement factor I (CFI), Complement C2 (C2), Complement Factor B (CFB), Complement C9 (C9), Mitochondrially Encoded TRNA Leucine 1 (UUA/G) (MT-TL-1), Complement Factor H Related 1 (CFHR1), Complement Factor H Related 3 (CFHR3), Ciliary Neurotrophic Factor (CNTF), pigment epithelium-derived factor (PEDF), rod-derived cone viability factor (RdCVF), glial-derived neurotrophic factor (GDNF), Myosin VIIA (MYO7A); Centrosomal Protein 290 (CEP290), Cadherin Related 23 (CDH23), Eyes Shut Homolog (EYS), Usherin (USH2A), adhesion G protein-coupled receptor V1 (ADGRV1), ALMS1 Centrosome And Basal Body Associated Protein (ALMS1), Retinoid Isomerohydrolase 65 kDa (RPE65), Aryl-hydrocarbon-interacting protein-like 1 (AIPL1), Guanylate Cyclase 2D, Retinal (GUCY2D), Leber Congenital Amaurosis 5 Protein (LCA5), Cone-Rod Homeobox (CRX), Clarin (CLRN1), ATP Binding Cassette Subfamily A Member 4 (ABCA4), Retinol Dehydrogenase 12 (RDH12), Inosine Monophosphate Dehydrogenase 1 (IMPDH1), Crumbs Cell Polarity Complex Component 1 (CRB1), Lecithin retinol acyltransferase (LRAT), Nicotinamide Nucleotide Adenylyltransferase 1 (NMNAT1), TUB Like Protein 1 (TULP1), MER Proto-Oncogene, Tyrosine Kinase (MERTK), Retinitis Pigmentosa GTPase Regulato (RPGR), RP2 Activator Of ARL3 GTPase (RP2), X-linked retinitis pigmentosa GTPase regulator-interacting protein 1 (RPGRIP), Cyclic Nucleotide Gated Channel Subunit Alpha 3 (CNGA3), Cyclic Nucleotide Gated Channel Subunit Beta 3 (CNGB3), G Protein Subunit Alpha Transducin 2 (GNAT2), Fibroblast Growth Factor 2 (FGF2), Erythropoietin (EPO), BCL2 Apoptosis Regulator (BCL2), BCL2 Like 1 (BCL2L1), Nuclear Factor Kappa B (NFκB), Endostatin, Angiostatin, fms-like tyrosine kinase receptor (sFlt), Pigment-dispersing factor receptor (Pdfr), Interleukin 10 (IL10), soluble interleukin 17 (sIL17R), Interleukin-1-receptor antagonist (IL1-ra), TNF Receptor Superfamily Member 1A (TNFRSF1A), TNF Receptor Superfamily Member 1B (TNFRSF1B), and interleukin 4 (IL4), optionally wherein the target gene is VEGFA.

27. (canceled)

28. The rAAV vector genome of claim 20, wherein the target RNA is a transcript (e.g., mRNA) of a target gene associated with a neurodegenerative disease or disorder, wherein the neurodegenerative disease or disorder is optionally alcoholism, Alexander's disease, Alper's disease, Alzheimer's Disease, amyotrophic lateral sclerosis (ALS), ataxia telangiectasia, neuronal ceroid lipofuscinoses, Batten disease, bovine spongiform encephalopathy (BSE), Canavan disease, cerebral palsy, Cockayne syndrome, corticobasal degeneration, Creutzfeldt-Jakob disease, frontotemporal lobar degeneration, Huntington's disease, HIV-associated dementia, Kennedy's disease, Lewy body dementia, neuroborreliosis, primary age-related tauopathy (PART)/Neurofibrillary tangle-predominant senile dementia, Machado-Joseph disease, multiple system atrophy, multiple sclerosis, multiple sulfatase deficiency, mucolipidoses, narcolepsy, Niemann Pick disease, Parkinson's Disease, Pick's disease, Pompe disease, primary lateral sclerosis, prion diseases, neuronal loss, cognitive defect, motor neuron diseases, Duchenne Muscular Dystrophy (DMD), frontotemporal dementia, frontotemporal dementia and parkinsonism linked to chromosome 17, Lytico-Bodig disease (Parkinson-dementia complex of Guam), neuroaxonal dystrophies, Refsum's disease, Schilder's disease, subacute combined degeneration of spinal cord secondary to pernicious anaemia, Spielmeyer-Vogt-Sjogren-Batten disease, Parkinsonism linked to chromosome 17 (FTDP-17), Prader Willi syndrome, Myotonic dystrophy, chronic traumatic encephalopathy including dementia pugilistica, spinocerebellar ataxia, spinal muscular atrophy, Steele-Richardson-Olszewski disease, Tabes dorsalis, Niemann-Pick Type C (NPC1 and/or NPC2 defect), Smith-Lemli-Opitz Syndrome (SLOS), an inborn error of cholesterol synthesis, Tangier disease, Pelizaeus-Merzbacher disease, a neuronal ceroid lipofuscinosis, a primary glycosphingolipidosis, Farber disease or multiple sulphatase deficiency, Gaucher disease, Fabry disease, GM1 gangliosidosis, GM2 gangliosidosis, Krabbe disease, metachromatic leukodystrophy (MLD), NPC, GM1 gangliosidosis, Fabry disease, a neurodegenerative mucopolysaccharidosis, MPS I, MPS IH, MPS IS, MPS II, MPS III, MPS IIIA, MPS IIIB, MPS IIIC, MPS HID, MPS, IV, MPS IV A, MPS IV B, MPS VI, MPS VII, MPS IX, a disease with secondary lysosomal involvement, SLOS, Tangier disease, ganglioglioma, gangliocytoma, meningioangiomatosis, postencephalitic parkinsonism, subacute sclerosing panencephalitis, lead encephalopathy, tuberous sclerosis, Hallervorden-Spatz disease, lipofuscinosis, cerebellar ataxia, parkinsonism, Louis-Barr syndrome, multiple systems atrophy, fronto-temporal dementia or lower body Parkinson's syndrome, Niemann Pick disease, Niemann Pick type C, Niemann Pick type A, Tay-Sachs disease, multisystemic atrophy cerebellar type (MSA-C), fronto-temporal dementia with parkinsonism, progressive supranuclear palsy, cerebellar downbeat nystagmus, Sandhoff's disease or mucolipidosis type II, or combinations thereof.

29. (canceled)

30. The rAAV vector genome of claim 20, wherein the target RNA is a transcript (e.g., mRNA) of a target gene associated with a cancer, wherein the cancer is optionally carcinomas, sarcomas, myelomas, leukemias, lymphomas and mixed type tumors, Non-limiting examples of cancers that may treated by methods and compositions described herein include, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus, In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor, nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular, mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; plasmacytoma, colorectal cancer, rectal cancer, and hairy cell leukemia.

31. (canceled)

32. The rAAV vector genome of claim 20, wherein said one or more sgRNAs comprise SEQ ID NOs: 7 and 8.

33. The rAAV vector genome of claim 1, comprising an ITR-to-ITR polynucleotide (such as SEQ ID NO: 17) comprising, from 5′ to 3′:
(a) a 5′ ITR from AAV2 (such as SEQ ID NO: 10);
(b) an EFS promoter (such as SEQ ID NO: 12);
(c) a KOZAK sequence (such as SEQ ID NO: 13);
(d) a first SV40 NLS coding sequence (such as SEQ ID NO: 14);
(e) the Cas13X polynucleotide (such as SEQ ID NO: 5) encoding the Cas13X polypeptide of SEQ ID NO: 2 or 3;
(f) a second SV40 NLS coding sequence (such as SEQ ID NO: 14);
(g) an SV40 polyA signal sequence (such as SEQ ID NO: 15);
(h) a U6 promoter (such as SEQ ID NO: 16);
(i) a first direct repeat sequence (such as SEQ ID NO: 6);
(j) an sg1 coding sequence specific for VEGFA (such as SEQ ID NO: 7);
(k) a second direct repeat sequence (such as SEQ ID NO: 6);
(l) an sg2 coding sequence specific for VEGFA (such as SEQ ID NO: 8);
(m) a third direct repeat sequence (such as SEQ ID NO: 6); and,
(n) a 3′ ITR from AAV2 (such as SEQ ID NO: 11);
or a polynucleotide at least about 90%, 91%, 92%, 93%, 94%, 95%, 96%, 97%, 98%, 99%, 99.5%, 99.6%, 99.7%, 99.8%, 99.9% identical to said ITR-to-ITR polynucleotide.

34. A recombinant AAV (rAAV) vector genome comprising, consisting essentially of, or consisting of SEQ ID NO: 17, or a polynucleotide at least 90%, 91%, 92%, 93%, 94%, 95%, 96%, 97%, 98%, 99%, 99.5%, 99.6%, 99.7%, 99.8%, 99.9% identical thereto, wherein said polynucleotide encodes a Cas13X polypeptide at least 80%, 85%, 90%, 95%, 96%, 97%, 98%, or 99% identical to SEQ ID NO: 4, and an sgRNA specific for VEGFA,
wherein said Cas13X polypeptide comprises 1-3 substitutions at Y672, Y676, and/or I751 of SEQ ID NO: 4, and
wherein said sgRNA forms a complex with said Cas13X polypeptide and directs said Cas13X polypeptide to cleave a VEGFA mRNA transcript with substantially the same (e.g., at least about 80%, 90%, 95%, 99% or more) guide RNA-specific nuclease activity as SEQ ID NO: 4 and substantially no (e.g., at most 20%, 15%, 10%, 5%) collateral (guide RNA-independent) nuclease activity of SEQ ID NO: 4.

35-36. (canceled)

37. A recombinant AAV (rAAV) viral particle comprising the rAAV vector genome of claim 1.

38-41. (canceled)

42. A pharmaceutical composition comprising the rAAV vector genome of claim 1, and a pharmaceutically acceptable excipient.

43. A method of treating a subject having an eye disease or disorder in a subject in need thereof, the method comprising administering to the subject a therapeutically effective amount of the rAAV vector genome of claim 1, wherein the rAAV vector genome specifically down-regulate the expression of a target gene causative of the eye disease or disorder.

44. The method of claim 43, wherein administrating comprises contacting a cell with the therapeutically effective amount of the rAAV vector genome, wherein the cell is optionally located in the eye of the subject.

45. (canceled)

46. The method of claim 43, wherein the eye disease or disorder is amoebic keratitis, fungal keratitis, bacterial keratitis, viral keratitis, onchorcercal keratitis, keratoconjunctivitis, bacterial keratoconjunctivitis, viral keratoconjunctivitis, vernal keratoconjunctivitis, atopic keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, noninfectious uveitis, anterior uveitis, posterior uveitis (including toxoplasmosis), pan-uveitis, an inflammatory disease of the vitreous or retina, endophthalmitis prophylaxis and treatment, macular edema, macular degeneration, wet age related macular degeneration (wet AMD), dry age related macular degeneration (dry AMD), diabetic macular edema (DME), allergic conjunctivitis, proliferative and non-proliferative diabetic retinopathy, hypertensive retinopathy, an autoimmune disease of the retina, primary and metastatic intraocular melanoma, other intraocular metastatic tumors, open angle glaucoma, Stargardt's disease, Fundus Flavimaculatus, closed angle glaucoma, pigmentary glaucoma, retinitis pigmentosa (RP), Leber's congenital amaurosis (LCA), Usher's syndrome, choroideremia, a rod-cone or cone-rod dystrophy, a ciliopathy, a mitochondrial disorder, progressive retinal atrophy, a degenerative retinal disease, geographic atrophy, a familial or acquired maculopathy, a retinal photoreceptor disease, a retinal pigment epithelial-based disease, cystoid macular edema, retinal detachment, traumatic retinal injury, iatrogenic retinal injury, macular holes, macular telangiectasia, a ganglion cell disease, an optic nerve cell disease, optic neuropathy, ischemic retinal disease, retinopathy of prematurity, retinal vascular occlusion, familial macroaneurysm, a retinal vascular disease, an ocular vascular diseases, a vascular disease, an ischemic optic neuropathy disease, diabetic retinal oedema, senile macular degeneration due to sub-retinal neovascularization, myopic retinopathy, retinal ischemia, choroidal vascular insufficiency, choroidal thrombosis and neovascular retinopathies resulting from carotoid artery ischemia, corneal neovascularisation, a corneal disease or opacification with an exudative or inflammatory component, diffuse lamellar keratitis, neovascularisation due to penetration of the eye or contusive ocular injury, rubosis iritis, Fuchs' heterochromic iridocyclitis, chronic uveitis, anterior uveitis, inflammatory conditions resulting from surgeries such as LASIK, LASEK, refractive surgery, IOL implantation; irreversible corneal oedema as a complication of cataract surgery, oedema as a result of insult or trauma, inflammation, infectious and non-infectious conjunctivitis, iridocyclitis, iritis, scleritis, episcleritis, superficial punctuate keratitis, keratoconus, posterior polymorphous dystrophy, Fuch's dystrophies, aphakic and pseudophakic bullous keratopathy, corneal oedema, scleral disease, ocular cicatrcial pemphigoid, pars planitis, Posner Schlossman syndrome, Behcet's disease, Vogt-Koyanagi-Harada syndrome, hypersensitivity reactions, ocular surface disorders, conjunctival oedema, Toxoplasmosis chorioretinitis, inflammatory pseudotumor of the orbit, chemosis, conjunctival venous congestion, periorbiatal cellulits, acute dacroycystitis, non-specific vasculitis, sarcoidosis, cytomegalovirus infection, and combinations thereof, optionally wherein the eye disease or disorder is wet age related macular degeneration (wet AMD).

47-48. (canceled)

49. The method of claim 43, wherein expression of the target gene in the cell is decreased in comparison to a cell having not been contacted with the rAAV vector genome, wherein the target gene is optionally VEGFA.

50-52. (canceled)

53. A non-human primate (NHP) model of wet AMD/CNV, comprising a NHP with an eye having developed laser-induced CNV, wherein the CNV is induced by laser photocoagulation about one month (e.g., about 3 weeks, 4 weeks, 31 days, or 5 weeks) after first administering one or more immunosuppressors to the NHP, and the laser-induced CNV persists at least about 4 weeks.

54-61. (canceled)

62. A method of identifying an inhibitor of wet AMD/CNV development or progression in the NHP model of wet AMD/CNV of claim 53, the method comprising contacting the retina of the NHP model of wet AMD/CNV with a candidate inhibitor, and determining the extent said candidate inhibitor inhibits the progression of CNV as compared to a vehicle control, wherein the candidate inhibitor that statistically significantly inhibits CNV progression compared to the vehicle control is selected as the inhibitor of wet AMD/CNV.

63-64. (canceled)
